SEQUENCE LISTING (228 sequences)

SEQ ID NO: 1 - protein, 19 aa, Artificial Sequence
  source: /note=“Description of Artificial Sequence: Synthetic peptide”
GSSYPMTARRFLEKHGLLD

SEQ ID NO: 2 - protein, 13 aa, Artificial Sequence
  source: /note=“Description of Artificial Sequence: Synthetic peptide”
GSSYPMTARRFLE

SEQ ID NO: 3 - protein, 19 aa, Artificial Sequence
  source: /note=“Description of Artificial Sequence: Synthetic peptide”
GSSYPMDARRFLEKHGLLD

SEQ ID NO: 4 - protein, 19 aa, Artificial Sequence
  source: /note=“Description of Artificial Sequence: Synthetic peptide”
GSSYPMTARRFLEKYGLLD

SEQ ID NO: 5 - protein, 19 aa, Artificial Sequence
  source: /note=“Description of Artificial Sequence: Synthetic peptide”
GSSYPMTARRFLEKWGLLR

SEQ ID NO: 6 - protein, 19 aa, Artificial Sequence
  source: /note=“Description of Artificial Sequence: Synthetic peptide”
GSSYPMTARRFLEKWGLLD

SEQ ID NO: 7 - protein, 18 aa, Artificial Sequence
  source: /note=“Description of Artificial Sequence: Synthetic peptide”
GSSYPMTARRFLEKHGLL

SEQ ID NO: 8 - protein, 17 aa, Artificial Sequence
  source: /note=“Description of Artificial Sequence: Synthetic peptide”
GSSYPMTARRFLEKHGL

SEQ ID NO: 9 - protein, 18 aa, Artificial Sequence
  source: /note=“Description of Artificial Sequence: Synthetic peptide”
SSYPMTARRFLEKHGLLD

SEQ ID NO: 10 - protein, 16 aa, Artificial Sequence
  source: /note=“Description of Artificial Sequence: Synthetic peptide”
YPMTARRFLEKHGLLD

SEQ ID NO: 11 - protein, 15 aa, Artificial Sequence
  source: /note=“Description of Artificial Sequence: Synthetic peptide”
YPMTARRFLEKHGLL

SEQ ID NO: 12 - protein, 14 aa, Artificial Sequence
  source: /note=“Description of Artificial Sequence: Synthetic peptide”
YPMTARRFLEKHGL

SEQ ID NO: 13 - protein, 19 aa, Artificial Sequence
  source: /note=“Description of Artificial Sequence: Synthetic peptide”
GSSYPMTARRFLEKHGLLR

SEQ ID NO: 14 - protein, 16 aa, Artificial Sequence
  source: /note=“Description of Artificial Sequence: Synthetic peptide”
YPMTARRFLEKWGLLR

SEQ ID NO: 15 - protein, 16 aa, Artificial Sequence
  source: /note=“Description of Artificial Sequence: Synthetic peptide”
YPMSARRFLAKWGLLR

SEQ ID NO: 16 - protein, 14 aa, Artificial Sequence
  source: /note=“Description of Artificial Sequence: Synthetic peptide”
APMTARRFLEKHGL

SEQ ID NO: 17 - protein, 14 aa, Artificial Sequence
  source: /note=“Description of Artificial Sequence: Synthetic peptide”
FPMTARRFLEKHGL

SEQ ID NO: 18 - protein, 14 aa, Artificial Sequence
  source: /note=“Description of Artificial Sequence: Synthetic peptide”
WPMTARRFLEKHGL

SEQ ID NO: 19 - protein, 14 aa, Artificial Sequence
  source: /note=“Description of Artificial Sequence: Synthetic peptide”
RPMTARRFLEKHGL

SEQ ID NO: 20 - protein, 14 aa, Artificial Sequence
  source: /note=“Description of Artificial Sequence: Synthetic peptide”
YAMTARRFLEKHGL

SEQ ID NO: 21 - protein, 14 aa, Artificial Sequence
  source: /note=“Description of Artificial Sequence: Synthetic peptide”
YPATARRFLEKHGL

SEQ ID NO: 22 - protein, 14 aa, Artificial Sequence
  source: /note=“Description of Artificial Sequence: Synthetic peptide”
YPLTARRFLEKHGL

SEQ ID NO: 23 - protein, 14 aa, Artificial Sequence
  source: /note=“Description of Artificial Sequence: Synthetic peptide”
YPFTARRFLEKHGL

SEQ ID NO: 24 - protein, 14 aa, Artificial Sequence
  source: /note=“Description of Artificial Sequence: Synthetic peptide”
YPWTARRFLEKHGL

SEQ ID NO: 25 - protein, 14 aa, Artificial Sequence
  source: /note=“Description of Artificial Sequence: Synthetic peptide”
YPMAARRFLEKHGL

SEQ ID NO: 26 - protein, 14 aa, Artificial Sequence
  source: /note=“Description of Artificial Sequence: Synthetic peptide”
YPMSARRFLEKHGL

SEQ ID NO: 27 - protein, 14 aa, Artificial Sequence
  source: /note=“Description of Artificial Sequence: Synthetic peptide”
YPMTYRRFLEKHGL

SEQ ID NO: 28 - protein, 14 aa, Artificial Sequence
  source: /note=“Description of Artificial Sequence: Synthetic peptide”
YPMTWRRFLEKHGL

SEQ ID NO: 29 - protein, 14 aa, Artificial Sequence
  source: /note=“Description of Artificial Sequence: Synthetic peptide”
YPMTRRRFLEKHGL

SEQ ID NO: 30 - protein, 14 aa, Artificial Sequence
  source: /note=“Description of Artificial Sequence: Synthetic peptide”
YPMTAKRFLEKHGL

SEQ ID NO: 31 - protein, 14 aa, Artificial Sequence
  source: /note=“Description of Artificial Sequence: Synthetic peptide”
YPMTARAFLEKHGL

SEQ ID NO: 32 - protein, 14 aa, Artificial Sequence
  source: /note=“Description of Artificial Sequence: Synthetic peptide”
YPMTARKFLEKHGL

SEQ ID NO: 33 - protein, 14 aa, Artificial Sequence
  source: /note=“Description of Artificial Sequence: Synthetic peptide”
YPMTARHFLEKHGL

SEQ ID NO: 34 - protein, 14 aa, Artificial Sequence
  source: /note=“Description of Artificial Sequence: Synthetic peptide”
YPMTARRWLEKHGL

SEQ ID NO: 35 - protein, 14 aa, Artificial Sequence
  source: /note=“Description of Artificial Sequence: Synthetic peptide”
YPMTARRFWEKHGL

SEQ ID NO: 36 - protein, 14 aa, Artificial Sequence
  source: /note=“Description of Artificial Sequence: Synthetic peptide”
YPMTARRFLAKHGL

SEQ ID NO: 37 - protein, 14 aa, Artificial Sequence
  source: /note=“Description of Artificial Sequence: Synthetic peptide”
YPMTARRFLSKHGL

SEQ ID NO: 38 - protein, 14 aa, Artificial Sequence
  source: /note=“Description of Artificial Sequence: Synthetic peptide”
YPMTARRFLRKHGL

SEQ ID NO: 39 - protein, 14 aa, Artificial Sequence
  source: /note=“Description of Artificial Sequence: Synthetic peptide”
YPMTARRFLEAHGL

SEQ ID NO: 40 - protein, 14 aa, Artificial Sequence
  source: /note=“Description of Artificial Sequence: Synthetic peptide”
YPMTARRFLEHHGL

SEQ ID NO: 41 - protein, 14 aa, Artificial Sequence
  source: /note=“Description of Artificial Sequence: Synthetic peptide”
YPMTARRFLERHGL

SEQ ID NO: 42 - protein, 14 aa, Artificial Sequence
  source: /note=“Description of Artificial Sequence: Synthetic peptide”
YPMTARRFLEKAGL

SEQ ID NO: 43 - protein, 14 aa, Artificial Sequence
  source: /note=“Description of Artificial Sequence: Synthetic peptide”
YPMTARRFLEKYGL

SEQ ID NO: 44 - protein, 14 aa, Artificial Sequence
  source: /note=“Description of Artificial Sequence: Synthetic peptide”
YPMTARRFLEKWGL

SEQ ID NO: 45 - protein, 14 aa, Artificial Sequence
  source: /note=“Description of Artificial Sequence: Synthetic peptide”
YPMTARRFLEKRGL

SEQ ID NO: 46 - protein, 14 aa, Artificial Sequence
  source: /note=“Description of Artificial Sequence: Synthetic peptide”
YPMTARRFLEKKGL

SEQ ID NO: 47 - protein, 14 aa, Artificial Sequence
  source: /note=“Description of Artificial Sequence: Synthetic peptide”
YPMTARRFLEKHGW

SEQ ID NO: 48 - protein, 16 aa, Artificial Sequence
  source: /note=“Description of Artificial Sequence: Synthetic peptide”
  MOD_RES (4)..(4): alloThr
YPMXARRFLEKWGLLR

SEQ ID NO: 49 - protein, 16 aa, Artificial Sequence
  source: /note=“Description of Artificial Sequence: Synthetic peptide”
YPMSARRFLEKWGLLR

SEQ ID NO: 50 - protein, 16 aa, Artificial Sequence
  source: /note=“Description of Artificial Sequence: Synthetic peptide”
YPMNARRFLEKWGLLR

SEQ ID NO: 51 - protein, 16 aa, Artificial Sequence
  source: /note=“Description of Artificial Sequence: Synthetic peptide”
  MOD_RES (4)..(4): Dap
YPMXARRFLEKWGLLR

SEQ ID NO: 52 - protein, 16 aa, Artificial Sequence
  source: /note=“Description of Artificial Sequence: Synthetic peptide”
  MOD_RES (5)..(5): Abu
YPMTXRRFLEKWGLLR

SEQ ID NO: 53 - protein, 16 aa, Artificial Sequence
  source: /note=“Description of Artificial Sequence: Synthetic peptide”
  MOD_RES (6)..(6): Orn
YPMTAXRFLEKWGLLR

SEQ ID NO: 54 - protein, 16 aa, Artificial Sequence
  source: /note=“Description of Artificial Sequence: Synthetic peptide”
YPMTAQRFLEKWGLLR

SEQ ID NO: 55 - protein, 16 aa, Artificial Sequence
  source: /note=“Description of Artificial Sequence: Synthetic peptide”
  MOD_RES (8)..(8): Bph
YPMTARRXLEKWGLLR

SEQ ID NO: 56 - protein, 16 aa, Artificial Sequence
  source: /note=“Description of Artificial Sequence: Synthetic peptide”
YPMTARRYLEKWGLLR

SEQ ID NO: 57 - protein, 16 aa, Artificial Sequence
  source: /note=“Description of Artificial Sequence: Synthetic peptide”
YPMTARRFLAKWGLLR

SEQ ID NO: 58 - protein, 16 aa, Artificial Sequence
  source: /note=“Description of Artificial Sequence: Synthetic peptide”
YPMTMRRFLEKWGLLR

SEQ ID NO: 59 - protein, 16 aa, Artificial Sequence
  source: /note=“Description of Artificial Sequence: Synthetic peptide”
YPMTFRRFLEKWGLLR

SEQ ID NO: 60 - protein, 16 aa, Artificial Sequence
  source: /note=“Description of Artificial Sequence: Synthetic peptide”
  MOD_RES (8)..(8): Nal1
YPMTARRXLEKWGLLR

SEQ ID NO: 61 - protein, 16 aa, Artificial Sequence
  source: /note=“Description of Artificial Sequence: Synthetic peptide”
KPMTARRFLEKWGLLR

SEQ ID NO: 62 - protein, 16 aa, Artificial Sequence
  source: /note=“Description of Artificial Sequence: Synthetic peptide”
  MOD_RES (3)..(3): Nle
YPXTARRFLEKWGLLR

SEQ ID NO: 63 - protein, 16 aa, Artificial Sequence
  source: /note=“Description of Artificial Sequence: Synthetic peptide”
  MOD_RES (4)..(4): Hse
YPMXARRFLEKWGLLR

SEQ ID NO: 64 - protein, 16 aa, Artificial Sequence
  source: /note=“Description of Artificial Sequence: Synthetic peptide”
  MOD_RES (5)..(5): NMeAla
YPMTXRRFLEKWGLLR

SEQ ID NO: 65 - protein, 16 aa, Artificial Sequence
  source: /note=“Description of Artificial Sequence: Synthetic peptide”
  MOD_RES (3)..(3): NMeMet
  MOD_RES (5)..(5): NMeAla
YPXTXRRFLEKWGLLR

SEQ ID NO: 66 - protein, 16 aa, Artificial Sequence
  source: /note=“Description of Artificial Sequence: Synthetic peptide”
  MOD_RES (1)..(1): Natural or non-natural amino acid residue
  MOD_RES (3)..(6): Natural or non-natural amino acid residue
  MOD_RES (8)..(8): Natural or non-natural amino acid residue
  MOD_RES (10)..(10): Natural or non-natural amino acid residue
  VARIANT (12)..(12): /replace=“His”
  SITE (1)..(16): /note=“Variant residues given in the sequence have no preference with respect to those in the annotations for variant positions”
  source: /note=“See specification as filed for detailed description of substitutions and preferred embodiments”
XPXXXXRXLXKWGLLR

SEQ ID NO: 67 - protein, 17 aa, Artificial Sequence
  source: /note=“Description of Artificial Sequence: Synthetic peptide”
SYPMTARRFLEKHGLLD

SEQ ID NO: 68 - protein, 17 aa, Artificial Sequence
  source: /note=“Description of Artificial Sequence: Synthetic peptide”
SYPMTARRFLEKWGLLR

SEQ ID NO: 69 - protein, 16 aa, Artificial Sequence
  source: /note=“Description of Artificial Sequence: Synthetic peptide”
  MOD_RES (4)..(4): Dap
YPMXFRRFLEKWGLLR

SEQ ID NO: 70 - protein, 16 aa, Artificial Sequence
  source: /note=“Description of Artificial Sequence: Synthetic peptide”
  MOD_RES (4)..(4): Dap
  MOD_RES (8)..(8): Nal1
YPMXMRRXLEKWGLLR

SEQ ID NO: 71 - protein, 16 aa, Artificial Sequence
  source: /note=“Description of Artificial Sequence: Synthetic peptide”
  MOD_RES (5)..(5): Ahp
YPMTXRRFLEKWGLLR

SEQ ID NO: 72 - protein, 16 aa, Artificial Sequence
  source: /note=“Description of Artificial Sequence: Synthetic peptide”
YPMTARRFLQKWGLLR

SEQ ID NO: 73 - protein, 14 aa, Artificial Sequence
  source: /note=“Description of Artificial Sequence: Synthetic peptide”
RPMTFRRFLEKWGL

SEQ ID NO: 74 - protein, 14 aa, Artificial Sequence
  source: /note=“Description of Artificial Sequence: Synthetic peptide”
  MOD_RES (1)..(1): Natural or non-natural amino acid residue
  MOD_RES (3)..(6): Natural or non-natural amino acid residue
  MOD_RES (8)..(8): Natural or non-natural amino acid residue
  MOD_RES (10)..(10): Natural or non-natural amino acid residue
  VARIANT (12)..(12): /replace=“His”
  SITE (1)..(14): /note=“Variant residues given in the sequence have no preference with respect to those in the annotations for variant positions”
  source: /note=“See specification as filed for detailed description of substitutions and preferred embodiments”
XPXXXXRXLXKWGL

SEQ ID NO: 75 - protein, 6 aa, Artificial Sequence
  source: /note=“Description of Artificial Sequence: Synthetic 6xHis tag”
HHHHHH

SEQ ID NO: 76 - protein, 8 aa, Escherichia coli
YPMTARRF

SEQ ID NO: 77 - protein, 4 aa, Escherichia coli
YPMT

SEQ ID NO: 78 - protein, 16 aa, Artificial Sequence
  source: /note=“Description of Artificial Sequence: Synthetic peptide”
  MOD_RES (4)..(4): Dap
YPMXMRRFLEKWGLLR

SEQ ID NO: 79 - protein, 16 aa, Artificial Sequence
  source: /note=“Description of Artificial Sequence: Synthetic peptide”
YPFTFRRFLEKWGLLR

SEQ ID NO: 80 - protein, 16 aa, Artificial Sequence
  source: /note=“Description of Artificial Sequence: Synthetic peptide”
  MOD_RES (4)..(4): Dap
  MOD_RES (8)..(8): Bph
YPMXARRXLEKWGLLR

SEQ ID NO: 81 - protein, 16 aa, Artificial Sequence
  source: /note=“Description of Artificial Sequence: Synthetic peptide”
  MOD_RES (4)..(4): Dap
  MOD_RES (8)..(8): Nal1
YPMXARRXLEKWGLLR

SEQ ID NO: 82 - protein, 16 aa, Artificial Sequence
  source: /note=“Description of Artificial Sequence: Synthetic peptide”
  MOD_RES (4)..(4): Dap
  MOD_RES (8)..(8): Bph
YPMXMRRXLEKWGLLR

SEQ ID NO: 83 - protein, 16 aa, Artificial Sequence
  source: /note=“Description of Artificial Sequence: Synthetic peptide”
YPMTLRRFLEKWGLLR

SEQ ID NO: 84 - protein, 16 aa, Artificial Sequence
  source: /note=“Description of Artificial Sequence: Synthetic peptide”
  MOD_RES (8)..(8): Hph
YPMTARRXLEKWGLLR

SEQ ID NO: 85 - protein, 16 aa, Artificial Sequence
  source: /note=“Description of Artificial Sequence: Synthetic peptide”
  MOD_RES (10)..(10): Hse
YPMTARRFLXKWGLLR

SEQ ID NO: 86 - protein, 16 aa, Artificial Sequence
  source: /note=“Description of Artificial Sequence: Synthetic peptide”
  MOD_RES (4)..(4): Dap
YPMXARRFLAKWGLLR

SEQ ID NO: 87 - protein, 16 aa, Artificial Sequence
  source: /note=“Description of Artificial Sequence: Synthetic peptide”
YPMTFRRFLAKWGLLR

SEQ ID NO: 88 - protein, 16 aa, Artificial Sequence
  source: /note=“Description of Artificial Sequence: Synthetic peptide”
  MOD_RES (8)..(8): Bph
YPMTARRXLAKWGLLR

SEQ ID NO: 89 - protein, 16 aa, Artificial Sequence
  source: /note=“Description of Artificial Sequence: Synthetic peptide”
  MOD_RES (4)..(4): Dap
YPMXMRRWLEKWGLLR

SEQ ID NO: 90 - protein, 16 aa, Artificial Sequence
  source: /note=“Description of Artificial Sequence: Synthetic peptide”
  MOD_RES (4)..(4): Dap
  MOD_RES (8)..(8): Bph
YPMXFRRXLEKWGLLR

SEQ ID NO: 91 - protein, 16 aa, Artificial Sequence
  source: /note=“Description of Artificial Sequence: Synthetic peptide”
  MOD_RES (3)..(3): Hph
YPXTARRFLEKWGLLR

SEQ ID NO: 92 - protein, 16 aa, Artificial Sequence
  source: /note=“Description of Artificial Sequence: Synthetic peptide”
  MOD_RES (5)..(5): D-Ala
YPMTXRRFLEKWGLLR

SEQ ID NO: 93 - protein, 16 aa, Artificial Sequence
  source: /note=“Description of Artificial Sequence: Synthetic peptide”
  MOD_RES (5)..(5): Aib
YPMTXRRFLEKWGLLR

SEQ ID NO: 94 - protein, 16 aa, Artificial Sequence
  source: /note=“Description of Artificial Sequence: Synthetic peptide”
YPMTARRFFEKWGLLR

SEQ ID NO: 95 - protein, 16 aa, Artificial Sequence
  source: /note=“Description of Artificial Sequence: Synthetic peptide”
  MOD_RES (3)..(3): NMeMet
  MOD_RES (4)..(4): Dap
YPXXFRRFLEKWGLLR

SEQ ID NO: 96 - protein, 16 aa, Artificial Sequence
  source: /note=“Description of Artificial Sequence: Synthetic peptide”
  MOD_RES (3)..(3): NMeMet
  MOD_RES (4)..(4): Dap
YPXXMRRFLEKWGLLR

SEQ ID NO: 97 - protein, 16 aa, Artificial Sequence
  source: /note=“Description of Artificial Sequence: Synthetic peptide”
  MOD_RES (3)..(3): NMeMet
  MOD_RES (4)..(4): Dap
  MOD_RES (5)..(5): NMeAla
YPXXXRRFLEKWGLLR

SEQ ID NO: 98 - protein, 16 aa, Artificial Sequence
  source: /note=“Description of Artificial Sequence: Synthetic peptide”
  MOD_RES (3)..(3): NMeMet
  MOD_RES (4)..(4): Dap
  MOD_RES (5)..(5): NMeMet
YPXXXRRFLEKWGLLR

SEQ ID NO: 99 - protein, 16 aa, Artificial Sequence
  source: /note=“Description of Artificial Sequence: Synthetic peptide”
  MOD_RES (3)..(3): NMePhe
  MOD_RES (5)..(5): NMeAla
YPXTXRRFLEKWGLLR

SEQ ID NO: 100 - protein, 16 aa, Artificial Sequence
  source: /note=“Description of Artificial Sequence: Synthetic peptide”
  MOD_RES (3)..(3): NMeMet
  MOD_RES (4)..(4): Dap
  MOD_RES (5)..(5): NMePhe
YPXXXRRFLEKWGLLR

SEQ ID NO: 101 - protein, 15 aa, Artificial Sequence
  source: /note=“Description of Artificial Sequence: Synthetic peptide”
YPMTARRFLEKHGGL

SEQ ID NO: 102 - protein, 16 aa, Artificial Sequence
  source: /note=“Description of Artificial Sequence: Synthetic peptide”
  MOD_RES (2)..(2): Dap
YXMPFRRFLEKWGLLR

SEQ ID NO: 103 - protein, 16 aa, Artificial Sequence
  source: /note=“Description of Artificial Sequence: Synthetic peptide”
  MOD_RES (3)..(3): Dap
YPXMFRRFLEKWGLLR

SEQ ID NO: 104 - protein, 16 aa, Artificial Sequence
  source: /note=“Description of Artificial Sequence: Synthetic peptide”
  MOD_RES (5)..(5): Dap
YPMFXRRFLEKWGLLR

SEQ ID NO: 105 - protein, 16 aa, Artificial Sequence
  source: /note=“Description of Artificial Sequence: Synthetic peptide”
  MOD_RES (6)..(6): Dap
YPMRFXRFLEKWGLLR

SEQ ID NO: 106 - protein, 16 aa, Artificial Sequence
  source: /note=“Description of Artificial Sequence: Synthetic peptide”
  MOD_RES (4)..(4): Dab
YPMXFRRFLEKWGLLR

SEQ ID NO: 107 - protein, 16 aa, Artificial Sequence
  source: /note=“Description of Artificial Sequence: Synthetic peptide”
YPMKFRRFLEKWGLLR

SEQ ID NO: 108 - protein, 16 aa, Artificial Sequence
  source: /note=“Description of Artificial Sequence: Synthetic peptide”
YPMRFRRFLEKWGLLR

SEQ ID NO: 109-199 - skipped (91 entries)

SEQ ID NO: 200 - protein, 14 aa, Artificial Sequence
  source: /note=“Description of Artificial Sequence: Synthetic peptide”
RPMTWRRFLAKYGL

SEQ ID NO: 201 - protein, 14 aa, Artificial Sequence
  source: /note=“Description of Artificial Sequence: Synthetic peptide”
RPMTARRWLAKRGL

SEQ ID NO: 202 - protein, 14 aa, Artificial Sequence
  source: /note=“Description of Artificial Sequence: Synthetic peptide”
RPMTARRWLEKRGL

SEQ ID NO: 203 - protein, 14 aa, Artificial Sequence
  source: /note=“Description of Artificial Sequence: Synthetic peptide”
RPMTWRRFLAKRGL

SEQ ID NO: 204 - protein, 14 aa, Artificial Sequence
  source: /note=“Description of Artificial Sequence: Synthetic peptide”
RPMTWRRWLEKHGL

SEQ ID NO: 205 - protein, 14 aa, Artificial Sequence
  source: /note=“Description of Artificial Sequence: Synthetic peptide”
RPMTWRRWLEKYGL

SEQ ID NO: 206 - protein, 14 aa, Artificial Sequence
  source: /note=“Description of Artificial Sequence: Synthetic peptide”
RPMTMRRFLEKWGL

SEQ ID NO: 207 - protein, 14 aa, Artificial Sequence
  source: /note=“Description of Artificial Sequence: Synthetic peptide”
  MOD_RES (8)..(8): Bph
RPMTARRXLEKWGL

SEQ ID NO: 208 - protein, 14 aa, Artificial Sequence
  source: /note=“Description of Artificial Sequence: Synthetic peptide”
  MOD_RES (8)..(8): Nal1
RPMTARRXLEKWGL

SEQ ID NO: 209 - protein, 14 aa, Artificial Sequence
  source: /note=“Description of Artificial Sequence: Synthetic peptide”
  MOD_RES (4)..(4): Dap
  MOD_RES (8)..(8): Bph
RPMXMRRXLEKWGL

SEQ ID NO: 210 - protein, 14 aa, Artificial Sequence
  source: /note=“Description of Artificial Sequence: Synthetic peptide”
RPMTARRFLAKRGL

SEQ ID NO: 211 - protein, 14 aa, Artificial Sequence
  source: /note=“Description of Artificial Sequence: Synthetic peptide”
  MOD_RES (10)..(10): Hse
RPMTARRWLXKRGL

SEQ ID NO: 212 - protein, 14 aa, Artificial Sequence
  source: /note=“Description of Artificial Sequence: Synthetic peptide”
  MOD_RES (5)..(5): Ahp
RPMTXRRWLEKRGL

SEQ ID NO: 213 - protein, 13 aa, Artificial Sequence
  source: /note=“Description of Artificial Sequence: Synthetic peptide”
  MOD_RES (5)..(5): Bph
RPMTXRWLEKRGL

SEQ ID NO: 214 - protein, 14 aa, Artificial Sequence
  source: /note=“Description of Artificial Sequence: Synthetic peptide”
RPMTSRRWLAKRGL

SEQ ID NO: 215 - protein, 14 aa, Artificial Sequence
  source: /note=“Description of Artificial Sequence: Synthetic peptide”
  MOD_RES (5)..(5): SerMe
RPMTXRRWLAKRGL

SEQ ID NO: 216 - protein, 14 aa, Artificial Sequence
  source: /note=“Description of Artificial Sequence: Synthetic peptide”
  MOD_RES (3)..(3): NMeMet
  MOD_RES (5)..(5): NMeAla
RPXTXRRWLAKRGL

SEQ ID NO: 217 - protein, 14 aa, Artificial Sequence
  source: /note=“Description of Artificial Sequence: Synthetic peptide”
  MOD_RES (4)..(4): Dap
  MOD_RES (8)..(8): Bph
RPMXMRRXLAKWGL

SEQ ID NO: 218 - protein, 14 aa, Artificial Sequence
  source: /note=“Description of Artificial Sequence: Synthetic peptide”
  MOD_RES (4)..(4): Dap
  MOD_RES (8)..(8): Bph
RPMXMRRXLEKRGL

SEQ ID NO: 219 - protein, 14 aa, Artificial Sequence
  source: /note=“Description of Artificial Sequence: Synthetic peptide”
  MOD_RES (4)..(4): Dap
  MOD_RES (8)..(8): Bph
RPMXMRRXLAKRGL

SEQ ID NO: 220 - protein, 14 aa, Artificial Sequence
  source: /note=“Description of Artificial Sequence: Synthetic peptide”
  MOD_RES (5)..(5): Aib
RPMTXRRWLAKRGL

SEQ ID NO: 221 - protein, 14 aa, Artificial Sequence
  source: /note=“Description of Artificial Sequence: Synthetic peptide”
  MOD_RES (2)..(2): Pip
RXMTARRWLAKRGL

SEQ ID NO: 222 - protein, 14 aa, Artificial Sequence
  source: /note=“Description of Artificial Sequence: Synthetic peptide”
  MOD_RES (3)..(3): Hph
RPXTARRWLAKRGL

SEQ ID NO: 223 - protein, 14 aa, Artificial Sequence
  source: /note=“Description of Artificial Sequence: Synthetic peptide”
  MOD_RES (12)..(12): Dab
RPMTARRWLAKXGL

SEQ ID NO: 224 - protein, 14 aa, Artificial Sequence
  source: /note=“Description of Artificial Sequence: Synthetic peptide”
  MOD_RES (12)..(12): Ahp
RPMTARRWLAKXGL

SEQ ID NO: 225 - protein, 14 aa, Artificial Sequence
  source: /note=“Description of Artificial Sequence: Synthetic peptide”
  MOD_RES (11)..(11): Dab
RPMTARRWLAXRGL

SEQ ID NO: 226 - protein, 14 aa, Artificial Sequence
  source: /note=“Description of Artificial Sequence: Synthetic peptide”
  MOD_RES (3)..(3): PhNva
  MOD_RES (4)..(4): Dap
  MOD_RES (8)..(8): Bph
RPXXMRRXLAKRGL

SEQ ID NO: 227 - protein, 14 aa, Artificial Sequence
  source: /note=“Description of Artificial Sequence: Synthetic peptide”
  MOD_RES (3)..(3): PhNva
  MOD_RES (4)..(4): Dap
RPXXMRRWLAKRGL

SEQ ID NO: 228 - protein, 14 aa, Artificial Sequence
  source: /note=“Description of Artificial Sequence: Synthetic peptide”
  MOD_RES (2)..(5): Natural or non-natural amino acid residue
  MOD_RES (8)..(8): Natural or non-natural amino acid residue
  MOD_RES (10)..(12): Natural or non-natural amino acid residue
  source: /note=“See specification as filed for detailed description of substitutions and preferred embodiments”
RXXXXRRXLXXXGL

(19) United States
(12) Patent Application Publication
(10) Pub. No.: US 2022/0135622 A1
(43) Pub. Date: May 5, 2022

(54) ANTIBACTERIAL PEPTIDES AND METHODS OF USE

(71) Applicant: Genentech, Inc., South San Francisco, CA (US)

(72) Inventors: Thomas CLAIRFEUILLE, South San Francisco, CA (US); Emily J. HANAN, South San Francisco, CA (US); Jian Mehr-Dean PAYANDEH, South San Francisco, CA (US); Steven Thomas RUTHERFORD, South San Francisco, CA (US); Benjamin Douglas SELLERS, South San Francisco, CA (US); Nicholas John SKELTON, South San Francisco, CA (US)

(73) Assignee: Genentech, Inc., South San Francisco, CA (US)

(21) Appl. No.: 17/434,283

(22) PCT Filed: Feb. 26, 2020
(86) PCT No.: PCT/US2020/019777

§ 371 Date: Aug. 26, 2021

Related U.S. Application Data
(60) Provisional application No. 62/811,769, filed on Feb. 28, 2019.
(60) Provisional application No. 62/912,509, filed on Oct. 8, 2019.

(51) Int. Cl. C07K 7/08; A61P 31/04

(52) U.S. Cl. CPC C07K 7/08; A61K 38/00; A61P 31/04

(57) ABSTRACT

The invention provides antibacterial compositions comprising peptides that bind to a lipopolysaccharide and methods of using the same.

### SEQUENCE LISTING

The instant application contains a Sequence Listing, which has been submitted electronically in ASCII format and is hereby incorporated by reference in its entirety. Said ASCII copy, created on Feb. 3, 2020, is named P35201-WO_SL.txt and is 50,047 bytes in size.

### FIELD OF THE INVENTION

The present invention relates to antibacterial peptides and methods of using the same.

### BACKGROUND

Lipopolysaccharide (LPS) resides in the outer membrane (OM) of Gram-negative bacteria where it is responsible for barrier function and immune modulation. LPS is the target of polymyxins (PMXs), last resort antibiotics whose clinical use is threatened by modifications to LPS that confer resistance. Clinical resistance to PMXs is increasing, signaling an urgent need for new antimicrobial strategies. A high-resolution crystal structure of the essential bacterial membrane protein PbgA (PhoPQ barrier gene A, YejM) in complex with LPS revealed an LPS-binding motif along the inner membrane. PbgA achieves direct LPS coordination primarily through backbone-mediated interactions to the lipid A core. There is a need for new PbgA-inspired synthetic peptides that can selectively bind to LPS and inhibit growth of diverse Gram-negative bacterial species, including polymyxin-resistant strains.

### SUMMARY

Provided herein are PbgA-inspired antibacterial peptides engineered to afford lipopolysaccharide-binding affinity and Gram-negative antibacterial properties through select amino acid substitutions. Further provided herein are methods of using the same for the treatment of Gram-negative bacterial infections including those resistant to other antibiotics.

One aspect provided herein is a peptide of Formula I:

R¹—X¹-Pro-X²—X³—X⁴—X⁵-Arg-X⁶-Leu-X⁷-Lys-X⁸-Gly-Leu-Leu-Arg-R²(SEQ ID NO: 66) (Formula I)

wherein, R¹, X¹, X², X³, X⁴, X⁵, X⁶, X⁷, X⁸ and R² are as described herein.

One aspect provided herein is a peptide of Formula II:

R¹—X¹-Pro-X²—X³—X⁴—X⁵-Arg-X⁶-Leu-X⁷-Lys-X⁸-Gly-Leu-R²(SEQ ID NO: 74) (Formula II)

wherein, R¹, X¹, X², X³, X⁴, X⁵, X⁶, X⁷, X⁸ and R² are as described herein.

In another aspect provided herein is a peptide of Formula III:

R¹(Arg-X^{a}—X^{b}—X^{c}—X^{d}-Arg-Arg-X^{e}-Leu-X^{f}—X^{g}—X^{h}-Gly-Leu-R²(SEQ ID NO: 228) (Formula III)

wherein, R¹, X^{a}, X^{b}, X^{c}, X^{d}, X^{e}, X^{f}, X^{g}, X^{h}, and R² are as described herein.

One aspect provided herein is a pharmaceutical composition comprising a peptide described herein, and a pharmaceutically acceptable excipient.

One aspect provided herein is a peptide of the present invention, for use as therapeutically active substance.

One aspect provided herein is the use of a peptide of described herein, for the therapeutic treatment of a bacterial infection.

One aspect provided herein is a peptide described herein, for the preparation of a medicament for the therapeutic treatment of a bacterial infection.

One aspect provided herein is a peptide described herein, for the therapeutic treatment of a bacterial infection. In some embodiments, the bacterial infection is caused by a Gram-negative bacterium.

One aspect provided herein is a method for the therapeutic treatment of a bacterial infection, which method comprises administering a therapeutically effective amount of a peptide described herein.

One aspect provided herein is a peptide described herein, conjugated to a therapeutic agent.

One aspect provided herein is a method of producing a peptide described herein, comprising chemically synthesizing the peptide.

One aspect provided herein is an isolated nucleic acid encoding a peptide described herein.

One aspect provided herein is an expression vector encoding a nucleic acid molecule encoding a peptide described herein.

One aspect provided herein is a cell comprising an expression vector encoding a peptide described herein.

One aspect provided herein is a method of producing a peptide described herein, comprising culturing a cell of the present invention and recovering the peptide from the cell culture.

One aspect provided herein is a method of producing a peptide described herein, comprising culturing the cell as described herein and recovering the peptide from the cell culture.

One aspect provided herein is a method of treating an individual having a bacterial infection comprising administering to the individual an effective amount of a peptide that binds to a lipopolysaccharide comprising an amino acid sequence having a homology of ≥50% with SEQ ID NO: 1.

### BRIEF DESCRIPTION OF THE FIGURES

FIG. 1 shows an exemplary lipopolysaccharide molecule.

FIG. 2 shows orthogonal views (left and center) of the E. coli PbgA crystal structure. Transmembrane domain (TMD), interfacial facial domain (IFD), and periplasmic domain (PD) are shown. The electrostatic surface potential (right) of PbgA highlights the “positive inside” topology rule and lines approximate boundaries of the membrane bilayer.

FIG. 3 shows an Fₒ-F_{c} map of PbgA showing extra electron density along the IM periplasmic leaflet (contoured at 2 σ).

FIG. 4 shows a close-in view of the Fₒ-F_{c} map of PbgA calculated prior to the inclusion of LPS into the final model (contoured at 8 σ and 2 σ). Final refined coordinates of LPS shown for reference.

FIG. 5 shows a conservation analysis calculated across 300 PbgA homologs mapped onto a surface representation of PbgA. LPS is shown as spheres for reference.

FIG. 6 shows the top view of the PbgA LPS-binding motif. Bonding interactions are shown as dashed lines. water molecules as spheres and LPS in stick representation.

FIG. 7 shows the front view of the PbgA a7 helix abutting the 1′-phospho-group of lipid A.

FIG. 8 shows a schematic of the synthetic lipid A binding (LAB) peptides (SEQ ID NOs: 1, 2, 3, and 5); N-terminal biotin-Gly-Ser not shown.

FIG. 9 shows interferometry measurements made from captured biotinylated LAB peptides (SEQ ID NOs: 1, 2, 3, and 5) upon presenting peptides to different concentrations of detergent solubilized lipids (LPS, phosphatidylethanolamine (PE), phosphatidylglycerol (PG), and cardiolipin (CL)).

FIG. 10 shows bacterial growth inhibition curves of select LAB peptides (SEQ ID NOs: 1-3) tested on Gram-negative and Gram-positive bacteria.

FIG. 11 shows bacterial growth inhibition (IC₅₀) of E. coli ΔwaaD+EDTA, E. coli ΔwaaD+EDTA+colistinR (ColR), and USA300+EDTA by SEQ ID NO: 1.

FIG. 12 shows bacterial growth inhibition (IC₅₀) of E. coli ΔwaaD+EDTA, E. coli ΔwaaD+EDTA+colistinR (ColR), and USA300+EDTA by SEQ ID NO: 3.

FIG. 13 shows bacterial growth inhibition (IC₅₀) of E. coli ΔwaaD+EDTA by SEQ ID NOs: 1, 4, and 6.

FIG. 14 shows bacterial growth inhibition (IC₅₀) of USA300+EDTA by SEQ ID NOs: 1, 4, and 6.

FIG. 15 shows bacterial growth inhibition (IC₅₀) of USA300, E. coli imp4213 and E. coli+FhuAΔC/A4L by SEQ ID NO: 5.

FIG. 16A shows colony forming units (CFUs) of E. coli K12 measured over time with LABv2.1 peptide and polymyxin B present. FIG. 16B shows a red blood cell lysis assay evaluated after 4 hrs in the presence of indicated compounds.

### DETAILED DESCRIPTION

Synthetic lipid A-binding (LAB) peptides were found to bind LPS selectively over membrane PLs in vitro, with a Kd approaching ˜50 sM. Provided herein is a class of antimicrobial peptides capable of inhibiting diverse strains of Gram-negative bacteria, including strains that are resistant to polymyxins (PMXs), our present-day antibiotics of last resort.

### Definitions

“Affinity” refers to the strength of the sum total of noncovalent interactions between a single binding site of a molecule (e.g., a peptide) and its binding partner (e.g., a lipopolysaccharide). Unless indicated otherwise, as used herein, “binding affinity” refers to intrinsic binding affinity which reflects a 1:1 interaction between members of a binding pair (e.g., peptide and LPS). The affinity of a molecule X for its partner Y can generally be represented by the dissociation constant (Kd). Affinity can be measured by common methods known in the art, including those described herein. Specific illustrative and exemplary methods for measuring binding affinity are described in the following.

An “effective amount” of an agent, e.g., a pharmaceutical composition, refers to an amount effective, at dosages and for periods of time necessary, to achieve the desired therapeutic or prophylactic result.

“Homology” with respect to a reference peptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611. Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from wvw.fasta.bioch.virginia.edu/fasta_www2/fasta_down.shtml or www.ebi.ac.ukiTools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “IC₅₀”, as used herein, refers to the concentration of a peptide described herein (e.g. a peptide that binds to a lipopolysaccharide) that is required for 50% inhibition of bacterial growth.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The term “label” when used herein refers to a detectable compound or composition which is conjugated directly or indirectly to the peptides described herein (e.g. peptides that bind to a lipopolysaccharide). The label may itself be detectable by itself (e.g., radioisotope labels or fluorescent labels) or, in the case of an enzymatic label, may catalyze chemical alteration of a substrate compound or composition which is detectable.

The term “lipopolysaccharide” or “LPS”, as used herein, refers to a component of the outer membrane of Gram-negative bacteria consisting of a polysaccharide and lipid A. The polysaccharide, which varies from one bacterial species to another, is made up of the 0-specific chain and the core.

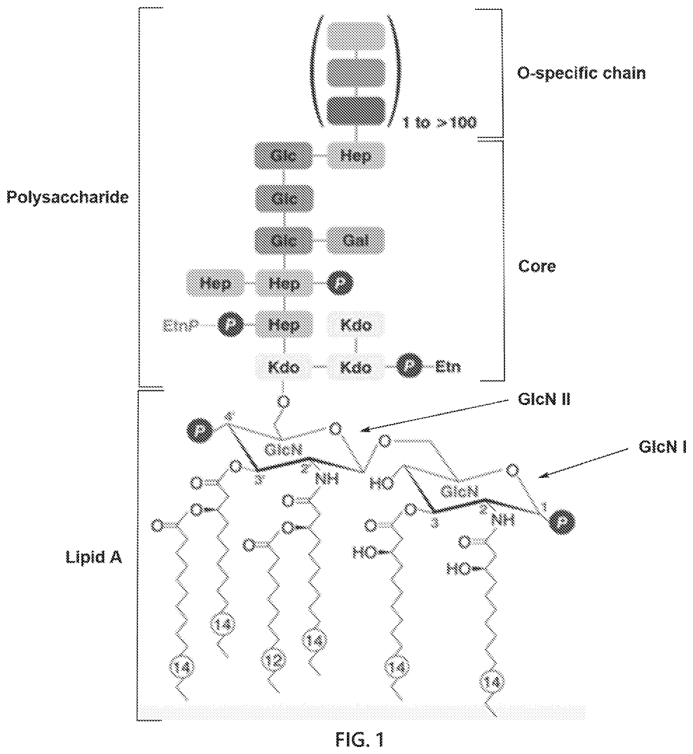

Lipid A is a unique and distinctive phosphoglycolipid, the structure of which is highly conserved among species. All contain glucosamine residues, which are present as β(1→6)-linked dimers. The disaccharide contains α-glycosidic and non-glycosidic phosphoryl groups in the 1 and 4′ positions, and (R)-3-hydroxy fatty acids at positions O-2, O-3, O-2′ and O-3′ in ester and amide linkages, of which two are usually further acylated at their 3-hydroxyl group. However, variations in the fine structure can arise from the type of hexosamine present, the degree of phosphorylation, the presence of phosphate substituents, and importantly in the nature, chain length, number, and position of the acyl groups. In the lipid A of the most studied organism Escherichia coli, the hydroxy fatty acids are C14 in chain length, and the hydroxy groups of the two (R)-3-hydroxy fatty acids of the distal GlcN-residue (GlcN II), and not those of the GlcN-residue at the reducing side (GlcN I), are acylated by fatty acids. Some molecular species contain an additional fatty acid attached to the amide-linked 3-hydroxy acid and the phosphate group may be substituted with ethanolamine-phosphate (of GlcN I). An exemplary LPS is shown in FIG. 1. See also, Rietschel and Brade, Scientific American August 1992, 54-61.

The term “minimum inhibitory concentration” or “MIC”, as used herein, refers to the lowest concentration of a peptide described herein (e.g. a peptide that binds to a lipopolysaccharide) that prevents visible bacterial growth.

The term “peptide”, as used herein, refers to an amino acid sequence between 2 and 100 amino acids in length, the amino acids being joined by peptide linkages. The amino acids may be naturally and non-naturally occurring.

The term “peptide that binds to a lipopolysaccharide” refers to a peptide that is capable of binding to a lipopolysaccharide. In certain aspects, a peptide that binds to lipopolysaccharide has a lipopolysaccharide-binding affinity in terms of the dissociation constant (Kd) of ≤1 mM, ≤100 μM, ≤10 μM, ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). With regard to the binding of a peptide described herein to a lipopolysaccharide, the term “selective binding” or “selectively binds to” or is “selective for” a lipopolysaccharide means that binding that is measurably different from a non-selective interaction. Selective binding can be measured, for example, by determining binding of a molecule compared to binding of a control molecule, which generally is a molecule of similar structure that does not have binding activity. For example, selective binding can be determined by competition with a control molecule that is similar to the target, for example, an excess of non-labeled target. In this case, selective binding is indicated if the binding of the labeled target to a probe is competitively inhibited by excess unlabeled target. In certain embodiments, the extent of binding of the peptide of the present disclosure to a “non-target” ligand will be less than about 10%) of the binding of the peptide described herein to a lipopolysaccharide as determined by, e.g., fluorescence activated cell sorting (FACS) analysis or radioimmunoprecipitation (RIA). In certain embodiments, a peptide of the present disclosure selectively binds to a target ligand (such as a lipopolysaccharide) with a dissociation constant (Kd) of ≤1 mM, ≤100 μM, ≤10 μM, ≤1 μM, 100 nM, ≤10 nM, ≤1 nM, 0.1 nM, 0.01 nM, or 0.001 nM (e.g., 10 M or less, e.g., from 10⁻⁸ M to 10⁻¹³ M, e.g., from 10⁻⁹ M to 10⁻¹³ M). In certain embodiments, the dissociation constant is measured at a temperature of about 4° C. 25° C., 37° C., or 45° C. In certain aspects, a peptide that binds to a lipopolysaccharide binds to a portion of a lipopolysaccharide that is conserved among lipopolysaccharides from different species. In certain aspects, a peptide that binds to a lipopolysaccharide binds to lipid A.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, peptides described herein (e.g. peptides that bind to a lipopolysaccharide) are used to delay development of a disease or to slow the progression of a disease.

### Amino Acid Abbreviations

Unless otherwise indicated, amino acid sequences are written left to right in amino to carboxy orientation.

| Amino Acid | Abbreviations |
|---|---|
| Glycine | Gly (G/g)* |
| Alanine | Ala (A/a)* |
| Serine | Ser (S/s)* |
| Threonine | Thr (T/t)* |
| Cysteine | Cys (C/c)* |
| Valine | Val (V/v)* |
| Leucine | Leu (L/l)* |
| Isoleucine | Ile (I/i)* |
| Methionine | Met (M/m)* |
| Proline | Pro (P/p)* |
| Phenylalanine | Phe (F/f)* |
| Tyrosine | Tyr (Y/y)* |
| Tryptophan | Trp (W/w)* |
| Aspartic Acid | Asp (D/d)* |
| Glutamic Acid | Glu (E/e)* |
| Asparagine | Asn (N/n)* |
| Glutamine | Gln (Q/q)* |
| Histidine | His (H/h)* |
| Lysine | Lys (K/k)* |
| Arginine | Arg (R/r)* |
| (S)-2-aminobutyric acid | Abu |
| (S)-3-([1,1′-biphenyl]4-yl)-2- | Bph |
| aminopropanoic acid | |
| (S)-2,3-diaminopropionie acid | Dap |
| homoserine | Hse |
| N-methylalanine | NMeAla |
| N-methylmethionine | NMeMet |
| (S)-2-amino-2-(naphthalen- | Nal1 |
| 1-yl)acetic acid | |
| Norleucine | Nle |
| Ornithine | Ora |
| homophenylalanine | Hph |
| 2-amino-2-methylpropanoic acid | Aib |
| N-methylphenyialartine | NMePhe |
| (S)-2-aminoheptanoic acid | Ahp |
| O-methyl-L-serine | SerMe |
| (S)-piperidine-2-carboxy1ic acid | Pip |
| (S)-2,3-diaminobutyric acid | Dab |
| (S)-2-amino-5- | PhNva |
| phenylpentanoic acid | |

*Upper case = L-amino acid;
Lower case = D-amino acid

### Compositions and Methods

In one aspect, peptides described herein are based, in part, on PbgA-inspired lipid A-binding (LAB) peptides. In certain aspects, peptides that can selectively coordinate LPS and kill diverse Gram-negative bacterial species in vitro, including PMX-resistance strains are provided. Peptides described herein (e.g. peptides that bind to a lipopolysaccharide) are useful, e.g., for the treatment of individuals having bacterial infections.

Table 1 shows exemplary sequences that are used throughout the application. Peptides are N-terminally acetylated and C-terminally amidated unless otherwise noted by “*”.

TABLE 1
Exemplary LAB Peptides
Sequence Listing

| SEQ ID NO: | Sequence |
|---|---|
| 1 | GSSYPMTARRFLEKHGLLD |
| 2 | GSSYPMTARRFLE |
| 3 | GSSYPMDARRFLEKHGLLD |
| 4 | GSSYPMTARRFLEKYGLLD |
| 5 | GSSYPMTARRFLEKWGLLR |
| 6 | GSSYPMTARRFLEKWGLLD |
| 7 | GSSYPMTARRFLEKHGLL |
| 8 | GSSYPMTARRFLEKHGL |
| 9 | SSYPMTARRFLEKHGLLD |
| 10 | YPMTARRFLEKHGLLD |
| 11 | YPMTARRFLEKHGLL |
| 12 | YMPTARRFLEKHGL |
| 13 | GSSYPMTARRFLEKHGLLR |
| 14 | YPMTARRFLEKWGLLR |
| 15 | YPMSARRFLAKWGLLR |
| 16 | APMTARRFLEKHGL |
| 17 | FPMTARRFLEKHGL |
| 18 | WPMTARRFLEKHGL |
| 19 | RPMTARRFLEKHGL |
| 20 | YAMTARRFLEKHGL |
| 21 | YPATARRFLEKHGL |
| 22 | YPLTARRFLEKHGL |
| 23 | YPFTARRFLEKHGL |
| 24 | YPWTARRFLEKHGL |
| 25 | YPMAARRFLEKHGL |
| 26 | YPMSARRFLEKHGL |
| 27 | YPMTYRRFLEKHGL |
| 28 | YPMTWRRFLEKHGL |
| 29 | YPMTRRRFLEKHGL |
| 30 | YPMTAKRFLEKHGL |
| 31 | YPMTARAFLEKHGL |
| 32 | YPMTARKFLEKHGL |
| 33 | YPMTARHFLEKHGL |
| 34 | YPMTARRWLEKHGL |
| 35 | YPMTARRFWEKHGL |
| 36 | YPMTARRFLAKHGL |
| 37 | YPMTARRFLSKHGL |
| 38 | YPMTARRFLRKHGL |
| 39 | YPMTARRFLEAHGL |
| 40 | YPMTARRFLEHHGL |
| 41 | YPMTARRFLERHGL |
| 42 | YPMTARRFLEKAGL |
| 43 | YPMTARRFLEKYGL |
| 44 | YPMTARRFLEKWGL |
| 45 | YPMTARRFLEKRGL |
| 46 | YPMTARRFLEKKGL |
| 47 | YPMTARRFLEKHGW |
| 48 | YPM(alloThr)ARRFLEKWGLLR |
| 49 | YPMSARRFLEKWGLLR |
| 50 | YPMNARRFLEKWGLLR |
| 51 | YPM(Dap)ARRFLEKWGLLR |
| 52 | WPMT(Abu)RRFLEKWGLLR |
| 53 | YPMTA(Orn)RFLEKWGLLR |
| 54 | YPMTAQRFLEKWGLLR |
| 55 | YPMTARR(Bph)LEKWGLLR |
| 56 | YPMTARRYLEKWGLLR |
| 57 | YPMTARRFLAKWGLLR |
| 58 | YPMTMRRFLEKWGLLR |
| 59 | YPMTFRRFLEKWGLLR |
| 60 | YPMTARR(Nal1)LEKWGLLR |
| 61 | KPMTARRFLEKWGLLR |
| 62 | YP(Nle)TARRFLEKWGLLR |
| 63 | YPM(Hse)ARRFLEKWGLLR |
| 64 | YPMT(NMeAla)RRFLEKWGLLR |
| 65 | YP(NMeMet)T(NMeAla)RRFLEKWGLLR |
| 66 | X¹ProX²X³X⁴X⁵ArgX⁶LeuX⁷LysX⁸GlyLeuLeuArg |
| 67 | SYPMTARRFLEKHGLLD |
| 68 | SYPMTARRFLEKWGLLR |
| 69 | YPM(Dap)FRRFLEKWGLLR |
| 70 | YPM(Dap)MRR(Nal1)LEKWGLLR |
| 71 | YPMT(Ahp)RRFLEKWGLLR |
| 72 | YPMTARRFLQKWGLLR |
| 73 | RPMTFRRFLEKWGL |
| 78 | YPM(Dap)MRRFLEKWGLLR |
| 79 | YPFTFRRFLEKWGLLR |
| 80 | YPM(Dap)ARR(Bph)LEKWGLLR |
| 81 | YPM(Dap)ARR(Nal1)LEKWGLLR |
| 82 | YPM(Dap)MRR(Bph)LEKWGLLR |
| 83 | YPMTLRRFLEKWGLLR |
| 84 | YPMTARR(Hph)LEKWGLLR |
| 85 | YPMTARRFL(Hse)KWGLLR |
| 86 | YPM(Dap)ARRFLAKWGLLR |
| 87 | YPMTFRRFLAKWGLLR |
| 88 | YPMTARR(Bph)LAKWGLLR |
| 89 | YPM(Dap)MRRWLEKWGLLR |
| 90 | YPM(Dap)FRR(Bph)LEKWGLLR |
| 91 | YP(Hph)TARRFLEKWGLLR |
| 92 | YPMTaRRFLEKWGLLR |
| 93 | YPMT(Aib)RRFLEKWGLLR |
| 94 | YPMTARRFFEKWGLLR |
| 95 | YP(NMeMet)(Dap)FRRFLEKWGLLR |
| 96 | YP(NMeMet)(Dap)MRRFLEKWGLLR |
| 97 | YP(NMeMet)(Dap)(NMeAla)RRFLEKWGLLR |
| 98 | YP(NMeMet)(Dap)(NMeMet)RRFLEKWGLLR |
| 99 | YP(NMePhe)T(NMeAla)RRFLEKWGLLR |
| 100 | YP(NMeMet)(Dap)(NMePhe)RRFLEKWGLLR |
| 101 | YPMTARRFLEKHGGL |
| 102 | Y(Dap)MPFRRFLEKWGLLR |
| 103 | YP(Dap)MFRRFLEKWGLLR |
| 104 | YPMF(Dap)RRFLEKWGLLR |
| 105 | YPMRF(Dap)RFLEKWGLLR |
| 106 | YPM(Dab)FRRFLEKWGLLR |
| 107 | YPMKFRRFLEKWGLLR |
| 108 | YPMRFRRFLEKWGLLR |
| 200 | RPMTWRRFLAKYGL |
| 201 | RPMTARRWLAKRGL |
| 202 | RPMTARRWLEKRGL |
| 203 | RPMTWRRFLAKRGL |
| 204 | RPMTWRRWLEKHGL |
| 205 | RPMTWRRWLEKYGL |
| 206 | RPMTMRRFLEKWGL |
| 207 | RPMTARR(Bph)LEKWGL |
| 208 | RPMTARR(Nal1)LEKWGL |
| 209 | RPM(Dap)MRR(Bph)LEKWGL |
| 210 | RPMTARRFLAKRGL |
| 211 | RPMTARRWL(Hse)KRGL |
| 212 | RPMT(Ahp)RRWLEKRGL |
| 213 | RPMT(Bph)RRWLEKRGL |
| 214 | RPMTSRRWLAKRGL |
| 215 | RPMT(SerMe)RRWLAKRGL |
| 216 | RP(NMeMet)T(NMeAla)RRWLAKRGL |
| 217 | RPM(Dap)MRR(Bph)LAKWGL |
| 218 | RPM(Dap)MRR(Bph)LEKRGL |
| 219 | RPM(Dap)MRR(Bph)LAKRGL |
| 220 | RPMT(Aib)RRWLAKRGL |
| 221 | R(Pip)MTARRWLAKRGL |
| 222 | RP(Hph)TARRWLAKRGL |
| 223 | RPMTARRWLAK(Dab)GL |
| 224 | RPMTARRWLAK(Ahp)GL |
| 225 | RPMTARRWLA(Dab)RGL |
| 226 | RP(PhNva)(Dap)MRR(Bph)LAKRGL |
| 227 | RP(PhNva)(Dap)MRRWLAKRGL |

One aspect provided herein is a peptide comprising an amino acid sequence selected from the group consisting of SEQ ID NOs: 5-65, 68-73, 78-108, and 200-227. In some embodiments, peptides include peptides comprising an amino acid sequence having a homology of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% homology to any of the amino acid sequences described herein and capable of binding to a lipopolysaccharide. In some embodiments, peptides include peptides comprising an amino acid sequence having a homology of at least 91%, at least 92%, at least 93%, at least 94%, at least 95%, at least 96%, at least 97%, at least 98%, or at least 99% homology to any of the amino acid sequences described herein and capable of binding to a lipopolysaccharide. In some embodiments, peptides include peptides comprising an amino acid sequence having a homology of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% homology to SEQ ID NO: 1 and having capable of binding to a lipopolysaccharide. In some embodiments, peptides include peptides comprising an amino acid sequence having a homology of at least 91%, at least 92%, at least 93%, at least 94%, at least 95%, at least 96%, at least 97%, at least 98%, or at least 99% homology to SEQ ID NO: 1 and having capable of binding to a lipopolysaccharide.

In some embodiments, peptides include analogs and derivatives that are modified, e.g., by the covalent attachment of any type of molecule that permits the peptide to retain its ability to bind to a lipopolysaccharide. For example, but not by way of limitation, derivatives and analogs of a peptide described herein include those that have been further modified, e.g., by glycosylation, acetylation, pegylation, phosphorylation, amidation, or derivatization by known protecting/blocking groups. In one embodiment, the modification is acetylation. In another embodiment, the modification is pegylation. Any of numerous chemical modifications can be carried out by known techniques. In some embodiments, peptides may be flanked by other amino acids such as cysteines, histidines or glycines, or amino acid sequences which do not destroy or interfere with the LPS-binding affinity or antibacterial activity of the peptides. In some embodiments, peptides may be attached to biomolecules or materials for binding, labeling or identification including biotin, streptavidin, oligonucleotides, other known sequence, peptides, nanoparticles, nanocrystals, nanospheres, polyethylene glycols, lipids, biomolecules, and the like. It is further contemplated that the peptides can be attached to the biomolecules through means of linking molecules or flanking amino acid sequence.

In one aspect provided herein are peptides that bind to a lipopolysaccharide. In one aspect, provided herein are isolated peptides that bind to a lipopolysaccharide. In one aspect provided herein are peptides that selectively bind to a lipopolysaccharide. In certain aspects, a peptide described herein that binds to a lipopolysaccharide has antibacterial activity.

One aspect provided herein are methods of treating an individual having a bacterial infection, the method comprising administering to the individual an effective amount of a peptide that binds to a lipopolysaccharide comprising an amino acid sequence having a homology of ≥50%, 75%, 85%, 90%, 95%, 97%, 98%, or 99% with SEQ ID NO: 1.

In some embodiments, a peptide described herein binds to a lipopolysaccharide of a Gram-negative bacterium. In some embodiments, the Gram-negative bacterium is selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp. In some embodiments, the Gram-negative bacterium is selected from the group consisting of Escherichia coli, Klebsiella spp., and Enterobacter spp. In some embodiments, the Gram-negative bacterium is selected from the group consisting of Escherichia coli, Klebsiella pneumoniae, Pseudomonas aeruginosa, Acinetobacter baumannii. Enterobacter cloacae, and Enterobacter aerogenes.

In some embodiments, the Gram-negative bacterium is selected from the group consisting of Escherichia coli, Klebsiella pneumoniae, Pseudomonas aeruginosa, Acinetobacter baumannii, and Enterobacter cloacae. In some embodiments, the Gram-negative bacterium is selected from the group consisting of Escherichia coli, Klebsiella pneumoniae, Enterobacter cloacae, and Enterobacter aerogenes. In some embodiments, the Gram-negative bacterium is selected from the group consisting of Escherichia coli, Klebsiella pneumoniae, Pseudomonas aeruginosa, and Acinetobacter baumanni.

In some embodiments, a peptide described herein binds to the lipid A portion of a lipopolysaccharide. In some embodiments, a peptide described herein has a lipopolysaccharide-binding affinity in terms of Kd of ≤1 mM, ≤100 μM, ≤10 μM, ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) as measured by biolayer interferometry. In some embodiments, a peptide described herein has a lipopolysaccharide-binding affinity in terms of Kd of about 1 mM to about 100 μM, about 100 μM to about 10 μM, about 10 μM to about 1 μM, about 1 μM to about 100 nM, about 100 nM to about 10 nM, about 10 nM to about 1 nM, about 1 nM to about 0.1 nM, about 0.1 nM to about 0.01 nM, or about 0.01 nM to about 0.001 nM, as measured by biolayer interferometry.

In some embodiments, a peptide described herein has a lipopolysaccharide-binding affinity in terms of Kd of ≤100 μM as measured by biolayer interferometry. In some embodiments, a peptide described herein has a lipopolysaccharide-binding affinity in terms of Kd of ≤10 μM as measured by biolayer interferometry. In some embodiments, a peptide described herein has a lipopolysaccharide-binding affinity in terms of Kd of ≤1 μM as measured by biolayer interferometry. In some embodiments, a peptide described herein binds to a lipopolysaccharide selectively over a bacterial membrane phospholipid. In some embodiments, the bacterial membrane phospholipid is phosphatidylethanolamine, phosphatidylglycerol, or cardiolipin.

In some embodiments, a peptide described herein has an IC₅₀ of 1 mM, ≤100 μM, ≤10 μM, ≤1 μM, ≤100 nM, ≤10 nM, or ≤1 nM, against a Gram-negative bacterium, as measured by an in vitro bacterial growth assay. In some embodiments, a peptide described herein has an IC₅₀ of about 1 mM to about 100 μM, about 100 μM to about 10 μM, about 10 μM to about 1 μM, about 1 μM to about 100 nM, about 100 nM to about 10 nM, or about 10 nM to about 1 nM, against a Gram-negative bacterium, as measured by an in vitro bacterial growth assay. In some embodiments, a peptide described herein has an IC₅₀ of ≤10 μM against a Gram-negative bacterium, as measured by an in vitro bacterial growth assay. In some embodiments, a peptide described herein has an IC₅₀ of ≤1 μM against a Gram-negative bacterium, as measured by an in vitro bacterial growth assay. In some embodiments, a peptide described herein has an IC₅₀ of ≤100 nM against a Gram-negative bacterium, as measured by an in vitro bacterial growth assay. In some embodiments, a peptide described herein has an MIC of ≤5 mM, ≤500 μM, ≤50 μM, ≤25 μM, ≤15 μM, ≤5 μM, ≤500 nM, ≤50 nM, or ≤5 nM, against a Gram-negative bacterium, as measured by an in vitro bacterial growth assay. In some embodiments, a peptide described herein has an MIC of ≤500 μM against a Gram-negative bacterium, as measured by an in vitro bacterial growth assay. In some embodiments, a peptide described herein has an MIC of ≤100 μM against a Gram-negative bacterium, as measured by an in vitro bacterial growth assay. In some embodiments, a peptide described herein has an MIC of ≤50 M against a Gram-negative bacterium, as measured by an in vitro bacterial growth assay. In some embodiments, a peptide described herein has an IC₅₀ of ≤100 nM against a Gram-negative bacterium, as measured by an in vitro bacterial growth assay. In some embodiments, the IC₅₀ is measured by an in vitro bacterial growth assay in LB or Mueller Hinton II cation-adjusted broth at 37° C.

In some embodiments, a peptide described herein has an MIC of about 5 mM to about 500 M, about 500 μM to about 50 M, about 50 μM to about 5 μM, about 5 μM to about 500 nM, about 500 nM to about 50 nM, or about 50 nM to about 5 nM, against a Gram-negative bacterium, as measured by an in vitro bacterial growth assay. In some embodiments, a peptide described herein has an MIC of ≤500 μM against a Gram-negative bacterium, as measured by an in vitro bacterial growth assay. In some embodiments, a peptide described herein has an MIC of ≤50 μM against a Gram-negative bacterium, as measured by an in vitro bacterial growth assay. In some embodiments, a peptide described herein has an MIC of ≤25 μM against a Gram-negative bacterium, as measured by an in vitro bacterial growth assay. In some embodiments, a peptide described herein has an MIC of ≤M against a Gram-negative bacterium, as measured by an in vitro bacterial growth assay. In some embodiments, a peptide described herein has an MIC of ≤5 μM against a Gram-negative bacterium, as measured by an in vitro bacterial growth assay. In some embodiments, the MIC is measured by an in vitro bacterial growth assay in LB or Mueller Hinton 11 cation-adjusted broth at 37° C.

In some embodiments, a peptide described herein has a length of 10-20 amino acid residues. In some embodiments, a peptide described herein has a length of 12-18 amino acid residues. In some embodiments, a peptide described herein has a length of 14-16 amino acid residues. In some embodiments, a peptide described herein comprises an amino acid sequence having a homology of 60% with SEQ ID NO: 1. In some embodiments, a peptide described herein comprises an amino acid sequence having a homology of ≥70% with SEQ ID NO: 1. In some embodiments, a peptide described herein comprises an amino acid sequence having a homology of 80% with SEQ ID NO: 1. In some embodiments, a peptide described herein comprises an amino acid sequence having a homology of ≥90% with SEQ ID NO: 1. In some embodiments, a peptide described herein comprises an amino acid sequence having a homology of ≥95% with SEQ ID NO: 1.

In some embodiments, a peptide described herein comprises an amino acid sequence selected from the group consisting of SEQ ID NOs: 14, 48, 49, 50, 51, 52, 53, 55, 57, 58, 59, 60, 62, 63, 64, and 65. In some embodiments, a peptide described herein comprises an amino acid sequence selected from the group consisting of SEQ ID NOs: 11, 12, 14, 48, 49, 50, 51, 52, 53, 55, 57, 58, 59, 60, 62, 63, 64, 65, 68, 69, 70, 71, 72, and 73. In another embodiment, a peptide described herein has amino acid sequence corresponding to SEQ ID NO: 11, 12, 14, 53, 58, 59, 60, 65, 68, 69, 70, 71, 72, or 73.

In some embodiments, a peptide described herein comprises an amino acid sequence corresponding to one of SEQ ID NOs: 11, 12, 14, 53, 58, 59, 60, 62, 65, or 68-73. In some embodiments, a peptide described herein comprises an amino acid sequence corresponding to one of SEQ ID NOs: 78-108. In some embodiments, a peptide described herein comprises an amino acid sequence corresponding to one of SEQ ID NOs: 78-101. In some embodiments, a peptide described herein comprises an amino acid sequence corresponding to one of SEQ ID NOs: 78-94. In some embodiments, a peptide described herein comprises an amino acid sequence corresponding to one of SEQ ID NOs: 78, 80, 81, 82, 86, 87, 88, 89, or 90.

In some embodiments, a peptide described herein comprises an amino acid sequence described herein or a peptide having at least 95%, 96%, 97%, 98%, or 99% thereto, having inhibitory action against E. coli. In some embodiments, a peptide described herein comprises an amino acid sequence described herein or a peptide having at least 95%, 96%, 97%, 98%, or 99% thereto, having inhibitory action against K. pneumonia. In some embodiments, a peptide described herein comprises an amino acid sequence described herein or a peptide having at least 95%, 96%, 97%, 98%, or 99% thereto, having inhibitory action against A. baumannii. In some embodiments, a peptide described herein comprises an amino acid sequence described herein or a peptide having at least 95%, 96%, 97%, 98%, or 99% thereto, having inhibitory action against P. aeruginosa.

In some embodiments, a peptide described herein comprises SEQ ID NO: 11. In some embodiments, a peptide described herein comprises SEQ ID NO: 12. In some embodiments, a peptide described herein comprises SEQ ID NO: 14. In some embodiments, a peptide described herein comprises SEQ ID NO: 48. In some embodiments, a peptide described herein comprises SEQ ID NO: 49. In some embodiments, a peptide described herein comprises SEQ ID NO: 50. In some embodiments, a peptide described herein comprises SEQ ID NO: 51. In some embodiments, a peptide described herein comprises SEQ ID NO: 52. In some embodiments, a peptide described herein comprises SEQ ID NO: 53. In some embodiments, a peptide described herein comprises SEQ ID NO: 55. In some embodiments, a peptide described herein comprises SEQ ID NO: 57. In some embodiments, a peptide described herein comprises SEQ ID NO: 58. In some embodiments, a peptide described herein comprises SEQ ID NO: 59. In some embodiments, a peptide described herein comprises SEQ ID NO: 60. In some embodiments, a peptide described herein comprises SEQ ID NO: 62. In some embodiments, a peptide described herein comprises SEQ ID NO: 63. In some embodiments, a peptide described herein comprises SEQ ID NO: 64. In some embodiments, a peptide described herein comprises SEQ ID NO: 65. In some embodiments, a peptide described herein comprises SEQ ID NO: 68. In some embodiments, a peptide described herein comprises SEQ ID NO: 69. In some embodiments, a peptide described herein comprises SEQ ID NO: 70. In some embodiments, a peptide described herein comprises SEQ ID NO: 71. In some embodiments, a peptide described herein comprises SEQ ID NO: 72. In some embodiments, a peptide described herein comprises SEQ ID NO: 73. In some embodiments, a peptide described herein comprises SEQ ID NO: 78. In some embodiments, a peptide described herein comprises SEQ ID NO: 80. In some embodiments, a peptide described herein comprises SEQ ID NO: 81. In some embodiments, a peptide described herein comprises SEQ ID NO: 82. In some embodiments, a peptide described herein comprises SEQ ID NO: 86. In some embodiments, a peptide described herein comprises SEQ ID NO: 87. In some embodiments, a peptide described herein comprises SEQ ID NO: 88. In some embodiments, a peptide described herein comprises SEQ ID NO: 89. In some embodiments, a peptide described herein comprises SEQ ID NO: 90.

In some embodiments, a peptide described herein comprises an amino acid sequence corresponding to one of SEQ ID NOs: 200-227. In some embodiments, a peptide described herein comprises an amino acid sequence corresponding to one of SEQ ID NOs: 200-216. In some embodiments, a peptide described herein comprises an amino acid sequence corresponding to one of SEQ ID NOs: 217-227. In some embodiments, a peptide described herein comprises SEQ ID NO: 200. In some embodiments, a peptide described herein comprises SEQ ID NO: 201. In some embodiments, a peptide described herein comprises SEQ ID NO: 202. In some embodiments, a peptide described herein comprises SEQ ID NO: 209. In some embodiments, a peptide described herein comprises SEQ ID NO: 211. In some embodiments, a peptide described herein comprises SEQ ID NO: 213. In some embodiments, a peptide described herein comprises SEQ ID NO: 217. In some embodiments, a peptide described herein comprises SEQ ID NO: 219. In some embodiments, a peptide described herein comprises SEQ ID NO: 222. In some embodiments, a peptide described herein comprises SEQ ID NO: 226. In some embodiments, a peptide described herein comprises SEQ ID NO: 227.

In some embodiments, the individual is human. In some embodiments, the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

One aspect provided herein are peptides of Formula I:

R¹—X¹-Pro-X²—X³—X⁴—X⁵-Arg-X⁶-Leu-X⁷-Lys-X⁸-Gly-Leu-Leu-Arg-R²(SEQ ID NO: 66) Formula I.

wherein,
R¹ is acetyl or is absent;
X¹, X², X³, X⁴, X⁵, X⁶, and X⁷ are each independently a natural or non-natural amino acid residue;
X⁸ is tryptophan or histidine; and
R² is amino or is absent.

In one embodiment, the C-terminal Arg attached to R² is Asp.

In another aspect provided herein is a peptide of Formula I wherein,
R¹ is acetyl or is absent;
X¹ is tyrosine, lysine, alanine, phenylalanine, tryptophan, or arginine;
X² is methionine, N-methylmethionine, norleucine, alanine, leucine, phenylalanine, N-methylphenylalanine, homophenylalanine, (S)-2,3-diaminopropionic acid, or tryptophan;
X³ is threonine, allo-threonine, serine, asparagine, (S)-2,3-diaminopropionic acid, (S)-2,3-diaminobutyric acid, homoserine, lysine, arginine, or alanine;
X⁴ is alanine, 2-aminobutyric acid, methionine, N-methylmethionine, phenylalanine, N-methylphenylalanine, N-methylalanine, tyrosine, (S)-2-aminoheptanoic acid, 2-amino-2-methylpropanoic acid, (S)-2,3-diaminopropionic acid, tryptophan, or arginine;
X⁵ is arginine, omithine, glutamine, 2-amino-2-methylpropanoic acid, (S)-2,3-diaminopropionic acid, or lysine;
X⁶ is phenylalanine, homophenylalanine, (S)-3-([1,1′-biphenyl]-4-yl)-2-aminopropanoic acid, (S)-2-amino-2-(naphthalen-1-yl)acetic acid, tyrosine, or tryptophan;
X⁷ is glutamic acid, glutamine, alanine, serine, homoserine, or arginine;
X is tryptophan or histidine; and
R² is amino or is absent.

In a further aspect provided herein are peptides having formula II:

R¹—X¹-Pro-X²—X³—X⁴—X⁵-Arg-X⁶-Leu-X⁷-Lys-X⁸-Gly-Leu-R²(SEQ ID NO: 74) (Formula II)

wherein, R¹, R², X¹, X², X³, X⁴, X⁵, X⁶, X⁷, X⁸ are as described herein.

In some embodiments, R¹ is acetyl. In some embodiments, R¹ is absent.

In some embodiments, X¹, X², X³, X⁴, X⁵, X⁶, and X⁷ are each independently selected from natural amino acids. In one embodiment, at least one of X¹, X², X³, X⁴, X⁵, X⁶, and X⁷ is a natural amino acid. In another embodiment, at least two of X¹, X², X³, X⁴, X⁵, X⁶, and X⁷ are a natural amino acid. In still another embodiment, at least three of X¹. X², X³, X⁴, X⁵, X⁶, and X⁷ are a natural amino acid. In still another embodiment, at least four of X¹, X², X³, X⁴, X⁵, X⁶, and X⁷ are a natural amino acid. In yet another embodiment, at least five of X¹, X², X³, X⁴, X⁵, X⁶, and X⁷ are a natural amino acid. In yet another embodiment, at least six of X¹, X², X³, X⁴, X⁵, X⁶, and X⁷ are a natural amino acid. In another embodiment. X¹, X², X³, X⁴, X⁵, X⁶, and X⁷ are independently a natural amino acid.

In another embodiment, at least one of X¹, X², X³, X⁴, X⁵, X⁶, and X⁷ is a non-natural amino acid residue. In another embodiment, at least two of X¹, X², X³, X⁴, X⁵, X⁶, and X⁷ are a non-natural amino acid residue. In still another embodiment, at least three of X¹, X², X³, X⁴. X⁵, X⁶, and X⁷ are a non-natural amino acid residue. In still another embodiment, at least four of X¹, X², X³, X⁴, X⁵, X⁶, and X⁷ are a non-natural amino acid residue.

In some embodiments, X¹ is a natural amino acid residue. In another embodiment, X¹ is a non-natural amino acid residue.

In some embodiments, X¹ is tyrosine or arginine. In another embodiment. X¹ is lysine, alanine, phenylalanine, tryptophan, or arginine.

In some embodiments, X¹ is tyrosine. In some embodiments, X¹ is lysine. In some embodiments, X¹ is alanine. In some embodiments, X¹ is phenylalanine. In some embodiments, X¹ is tryptophan. In some embodiments, X¹ is arginine.

In some embodiments, X² is a natural and non-natural amino acid residue. In another embodiment, X² is a non-natural amino acid residue.

In some embodiments, X² is methionine, N-methylmethionine, alanine, leucine, phenylalanine, or tryptophan. In another embodiment, X² is methionine, norleucine, alanine, leucine, phenylalanine, or tryptophan. In another embodiment, X² is methionine or N-methylmethionine.

In some embodiments, X² is methionine. In some embodiments, X² is N-methylmethionine. In some embodiments, X² is norleucine. In some embodiments, X² is alanine. In some embodiments, X² is leucine. In some embodiments, X² is phenylalanine. In some embodiments, X² is tryptophan.

In some embodiments, X² is N-methylphenylalanine, homophenylalanine, or (S)-2,3-diaminopropionic acid. In some embodiments, X² is N-methylphenylalanine. In some embodiments, X² is homophenylalanine. In some embodiments, X² is (S)-2,3-diaminopropionic acid.

In some embodiments, X³ is a natural amino acid residue. In another embodiment, X³ is a non-natural amino acid residue. In one embodiment, X³ is threonine or 2,3-diaminopropionic acid. In some embodiments, X³ is threonine, allo-threonine, serine, 2,3-diaminopropionic acid, homoserine, or alanine. In some embodiments, X³ is lysine or arginine.

In some embodiments, X³ is threonine. In some embodiments, X³ is allo-threonine. In some embodiments, X³ is serine. In some embodiments, X³ is asparagine. In some embodiments, X³ is 2,3-diaminopropionic acid. In some embodiments, X³ is homoserine. In some embodiments, X³ is alanine. In some embodiments. X³ is (S)-2,3-diaminobutyric acid.

In some embodiments, X⁴ is a natural amino acid residue. In another embodiment, X⁴ is a non-natural amino acid residue. In another embodiment, X⁴ is methionine phenylalanine, alanine, or N-methylalanine. In some embodiments, X⁴ is alanine, 2-aminobutyric acid, methionine, phenylalanine, N-methylalanine, tyrosine, or tryptophan.

In some embodiments, X⁴ is alanine. In some embodiments, X⁴ is 2-aminobutyric acid. In some embodiments, X⁴ is methionine. In some embodiments, X⁴ is phenylalanine. In some embodiments, X⁴ is N-methylalanine. In some embodiments, X⁴ is tyrosine. In some embodiments, X⁴ is tryptophan. In some embodiments, X⁴ is arginine.

In some embodiments, X⁴ is N-methylmethionine, N-methylphenylalanine, 2-amino-2-methylpropanoic acid, or (S)-2,3-diaminopropionic acid. In some embodiments, X⁴ is N-methylmethionine. In some embodiments, X⁴ is N-methylphenylalanine. In some embodiments, X⁴ is 2-amino-2-methylpropanoic acid. In some embodiments, X⁴ is (S)-2,3-diaminopropionic acid.

In some embodiments, X⁵ is a natural amino acid residue. In another embodiment, X⁵ is a non-natural amino acid residue. In some embodiments, X⁵ is arginine or omithine. In another embodiment, X⁵ is glutamine or lysine. In some embodiments. X⁵ is (S)-2,3-diaminopropionic acid or 2-amino-2-methylpropanoic acid.

In some embodiments, X⁵ is arginine. In some embodiments, X⁵ is omithine. In some embodiments, X⁵ is glutamine. In some embodiments, X⁵ is lysine.

In some embodiments, X⁶ is a natural amino acid residue. In another embodiment, X⁶ is a non-natural amino acid residue. In some embodiments, X⁶ is phenylalanine, (S)-3-([1,1′-biphenyl]-4-yl)-2-aminopropanoic (Bpa) or (S)-2-amino-2-(naphthalen-1-yl)acetic acid. In another embodiment, X⁶ is tyrosine or tryptophan.

In some embodiments, X⁶ is phenylalanine. In some embodiments, X⁶ is (S)-3-([1,1′-biphenyl]-4-yl)-2-aminopropanoic (Bpa). In some embodiments, X⁶ is (S)-2-amino-2-(naphthalen-1-yl)acetic acid. In some embodiments, X⁶ is tyrosine. In some embodiments, X⁶ is tryptophan. In some embodiments, X⁶ is homophenylalanine.

In some embodiments, X⁷ is a natural amino acid residue. In another embodiment, X⁷ is a non-natural amino acid residue. In some embodiments, X⁷ is glutamic acid, alanine, serine, and arginine. In another embodiment, X⁷ is glutamic acid or glutamine. In some embodiments, X⁷ is serine or homoserine. In some embodiments. X⁷ is homoserine.

In some embodiments, X⁷ is glutamic acid. In some embodiments, X⁷ is alanine. In some embodiments, X⁷ is serine. In some embodiments, X⁷ is arginine. In some embodiments, X⁷ is glutamine.

In some embodiments, X⁸ is tryptophan. In some embodiments, X⁸ is histidine.

In some embodiments, R² is amino. In some embodiments, R² is absent.

In one embodiment, the peptide of formula I or formula II comprises an amino acid sequence corresponding to SEQ ID NO: 5-65, 68-73, or 78-108. In one embodiment, the peptide of formula I or formula II comprises an amino acid sequence having at least 95%, 96%, 97%, 98% or 99% homology to a peptide corresponding to SEQ ID NO: 5-65, 68-73, 78-108.

Also provided herein are peptides of Formula III:

R¹-Arg-X^{a}—X^{b}—X^{c}—X^{d}-Arg-Arg-X^{e}-Leu-X^{f}—X^{g}—X^{h}-Gly-Leu-R²(SEQ ID NO: 228) (Formula III)

wherein,
R¹ is acetyl or is absent;
X^{a}, X^{b}, X^{c}, X^{d}, X^{e}, X^{f}, X^{g} and X^{h} are each independently a natural or non-natural amino acid residue; and
R² is amino or is absent.

In another aspect provided herein is a peptide of Formula III wherein,
R¹ is acetyl or is absent;
X^{a} is proline or (S)-piperidine-2-carboxylic acid;
X^{b} is methionine, N-methylmethionine, homophenylalanine or (S)-2-amino-5-phenylpentanoic acid;
X^{c} is threonine or (S)-2,3-diaminopropionic acid;
X^{d} is tryptophan, alanine, serine, methionine, (S)-2-aminoheptanoic acid, (S)-3-([1,1′-biphenyl]-4-yl)-2-aminopropanoic acid, O-methyl-L-serine, N-methylalanine, or 2-amino-2-methylpropanoic acid;
X^{e} is phenylalanine, tryptophan, (S)-3-([1,1′-biphenyl]-4-yl)-2-aminopropanoic acid, or (S)-2-amino-2-(naphthalen-1-yl)acetic acid;
X^{f} is alanine, glutamic acid, or homoserine;
X^{g} is lysine or (S)-2,3-diaminobutyric acid;
X^{h} is Arg, tyrosine, histidine, tryptophan, (S)-2,3-diaminobutyric acid, or (S)-2-aminoheptanoic acid; and
R² is amino or is absent.

In some embodiments, X^{a}, X^{b}, X^{c}, X^{d}, X^{e}, X^{f}, X^{g}, and X^{h} are each independently selected from natural amino acids. In one embodiment, at least one of X^{a}, X^{b}, X^{c}, X^{d}, X^{e}, X^{f}, X^{g}, and X^{h} is a natural amino acid. In another embodiment, at least two of X^{a}, X^{b}, X^{c}, X^{d}, X^{e}, X^{f}, X^{g}, and X^{h} are a natural amino acid. In still another embodiment, at least three of X^{a}, X^{b}, X^{c}, X^{d}, X^{e}, X^{f}, X^{g}, and X^{h} are a natural amino acid. In still another embodiment, at least four of X^{a}, X^{b}, X^{c}, X^{d}, X^{e}, X^{f}, X^{g}, and X^{h} are a natural amino acid. In yet another embodiment, at least five of X^{a}, X^{b}, X^{c}, X^{d}, X^{e}, X^{f}, X^{g}, and X^{h} are a natural amino acid. In yet another embodiment, at least six of X^{a}, X^{b}, X^{c}, X^{d}, X^{e}, X^{f}, X^{g}, and X^{h} are a natural amino acid. In yet another embodiment, at least seven of X^{a}, X^{b}, X^{c}, X^{d}, X^{e}, X^{f}, X^{g}, and X^{h} are a natural amino acid. In another embodiment, X^{a}, X^{b}, X^{c}, X^{d}, X^{e}, X^{f}, X^{g}, and X^{h} independently a natural amino acid.

In another embodiment, at least one of X^{a}, X^{b}, X^{c}, X^{d}, X^{e}, X^{f}, X^{g}, and X^{h} is a non-natural amino acid residue. In another embodiment, at least two of X^{a}, X^{b}, X^{c}, X^{d}, X^{e}, X^{f}, X^{g}, and X^{h} are a non-natural amino acid residue. In still another embodiment, at least three of X^{a}, X^{b}, X^{c}, X^{d}, X^{e}, X^{f}, X^{g}, and X^{h} are a non-natural amino acid residue. In still another embodiment, at least four of X^{a}, X^{b}, X^{c}, X^{d}, X^{e}, X^{f}, X^{g}, and X^{h} are a non-natural amino acid residue.

In some embodiments, X is a natural amino acid residue. In another embodiment. X^{a} is a non-natural amino acid residue. In some embodiments, X^{a} is proline. In some embodiments, X^{a} is (S)-piperidine-2-carboxylic acid.

In some embodiments, X^{b} is a natural amino acid residue. In another embodiment, X^{b} is a non-natural amino acid residue. In some embodiments, X^{b} is methionine. In some embodiments, X^{b} is N-methylmethionine, homophenylalanine, or (S)-2-amino-5-phenylpentanoic acid. In some embodiments, X^{b} is N-methylmethionine. In some embodiments, X^{b} is homophenylalanine. In some embodiments, X^{b} is (S)-2-amino-5-phenylpentanoic acid.

In some embodiments, X^{c} is a natural amino acid residue. In another embodiment, X^{c} is a non-natural amino acid residue. In some embodiments. X^{c} is threonine. In some embodiments, X^{c} is (S)-2,3-diaminopropionic acid.

In some embodiments, X^{d} is a natural amino acid residue. In another embodiment, X^{d} is a non-natural amino acid residue. In some embodiments, X^{d} is tryptophan, alanine, serine, or methionine. In some embodiments, X^{d} is tryptophan or alanine. In some embodiments, X^{d} is tryptophan. In some embodiments, X^{d} is alanine. In some embodiments, X^{d} is serine. In some embodiments, X^{d} is methionine. In some embodiments. X^{d} is (S)-2-aminoheptanoic acid, (S)-3-([1,1′-biphenyl]-4-yl)-2-aminopropanoic acid, O-methyl-L-serine, N-methylphenylalanine, or 2-amino-2-methylpropanoic acid. In some embodiments, X^{d} is (S)-2-aminoheptanoic acid. In some embodiments, X^{d} is (S)-3-([1,1′-biphenyl]-4-yl)-2-aminopropanoic acid. In some embodiments, X^{d} is O-methyl-L-serine. In some embodiments, X^{d} is N-methylphenylalanine. In some embodiments, X^{d} is 2-amino-2-methylpropanoic acid.

In some embodiments, X^{e} is a natural amino acid residue. In another embodiment, X^{e} is a non-natural amino acid residue. In some embodiments, X^{e} is phenylalanine or tryptophan. In some embodiments, X^{e} is phenylalanine. In some embodiments, X^{e} is tryptophan. In some embodiments, X^{e} is (S)-3-([1,1′-biphenyl]-4-yl)-2-aminopropanoic acid or (S)-2-amino-2-(naphthalen-1-yl)acetic acid. In some embodiments, X^{e} is (S)-3-([1,1′-biphenyl]-4-yl)-2-aminopropanoic acid. In some embodiments, X^{e} is (S)-2-amino-2-(naphthalen-1-yl)acetic acid.

In some embodiments, X^{f} is a natural amino acid residue. In another embodiment, X^{f} is a non-natural amino acid residue. In some embodiments, X^{f} is alanine or glutamic acid. In some embodiments, X^{f} is alanine. In some embodiments, X^{f} is glutamic acid. In some embodiments, X^{f} is homoserine.

In some embodiments, X is a natural amino acid residue. In another embodiment, X^{g} is a non-natural amino acid residue. In some embodiments, X^{g} is lysine. In some embodiments, X^{g} is (S)-2,3-diaminobutyric acid.

In some embodiments, X^{h} is a natural amino acid residue. In another embodiment, X^{h} is a non-natural amino acid residue. In some embodiments, X^{h} is arginine, tyrosine, histidine, or tryptophan. In some embodiments, X^{h} is arginine. In some embodiments. X^{h} is arginine or tryptophan. In some embodiments, X^{h} is tyrosine, histidine, or tryptophan. In some embodiments, X^{h} is tryptophan. In some embodiments, X^{h} is tyrosine. In some embodiments, X^{h} is histidine. In some embodiments, X^{h} is (S)-2,3-diaminobutyric acid or (S)-2-aminoheptanoic acid. In some embodiments, X^{h} is (S)-2,3-diaminobutyric acid. In some embodiments, X^{h} is (S)-2-aminoheptanoic acid.

In one embodiment, the peptide of formula III comprises an amino acid sequence corresponding to SEQ ID NO: 200-227. In one embodiment, the peptide of formula III comprises an amino acid sequence having at least 95%, 96%, 97%, 98% or 99% homology to a peptide corresponding to SEQ ID NO: 200-227.

Further provided herein are pharmaceutical compositions comprising a peptide described herein (e.g. a peptide that binds to a lipopolysaccharide), and at least one pharmaceutically acceptable excipient. In some embodiments, the pharmaceutical composition further comprises an additional therapeutic agent. In some embodiments, the additional therapeutic agent is an antibiotic or an antiseptic.

One aspect provided herein are methods for the therapeutic treatment of a bacterial infection, which method comprises administering a therapeutically effective amount of a peptide described herein (e.g. a peptide that binds to a lipopolysaccharide) having at least 90, 95, 97, 98, 99 percent homology to SEQ ID NO: 1 or to SEQ ID NO: 68. One aspect provided herein are methods for the therapeutic treatment of a bacterial infection, which method comprises administering a therapeutically effective amount of a peptide described herein (e.g. a peptide that binds to a lipopolysaccharide). One aspect provided herein are methods for the therapeutic treatment of a bacterial infection, which method comprises administering a therapeutically effective amount of a peptide having at least 95%, 96%, 97%, 98% or 99% homology to a peptide described herein (e.g. a peptide that binds to a lipopolysaccharide).

In some embodiments, the method further comprises administering an additional therapeutic agent. In some embodiments, the additional therapeutic agent comprises antibiotics or antiseptics.

In some embodiments, the bacterial infection is caused by a Gram-negative bacterium. In some embodiments, the bacterial infection is caused by a Gram-negative bacterium selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylon, Legionella spp., and Vibrio spp. In some embodiments, the bacterial infection is caused by a Gram-negative bacterium selected from the group consisting of Escherichia coli, Klebsiella spp., and Enterobacter spp.

In some embodiments, the bacterial infection is caused by a Gram-negative bacterium selected from the group consisting of Escherichia coli, Klebsiella pneumoniae, Pseudomonas aeruginosa, Acinetobacter baumannii, Enterobacter cloacae, and Enterobacter aerogenes. In some embodiments, the bacterial infection is caused by a Gram-negative bacterium selected from the group consisting of Escherichia coli, Klebsiella pneumoniae, Pseudomonas aeruginosa, Acinetobacter baumannii, and Enterobacter cloacae. In some embodiments, the bacterial infection is caused by a Gram-negative bacterium selected from the group consisting of Escherichia coli, Klebsiella pneumoniae, Enterobacter cloacae, and Enterobacter aerogenes. In some embodiments, the bacterial infection is caused by a Gram-negative bacterium selected from the group consisting of Escherichia coli. Klebsiella pneumoniae, Pseudomonas aeruginosa, and Acinetobacter baumannii.

In some embodiments, the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

In some embodiments, a peptide described herein binds to a lipopolysaccharide of a Gram-negative bacterium. In some embodiments, the Gram-negative bacterium is selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp. In some embodiments, the Gram-negative bacterium is selected from the group consisting of Escherichia coli, Klebsiella spp., and Enterobacter spp. In some embodiments, the Gram-negative bacterium is selected from the group consisting of Escherichia coli, Klebsiella pneumoniae, Pseudomonas aeruginosa, Acinetobacter baumannii, Enterobacter cloacae, and Enterobacter aerogenes. In some embodiments, the Gram-negative bacterium is selected from the group consisting of Escherichia coli, Klebsiella pneumoniae, Pseudomonas aeruginosa, Acinetobacter baumannii, and Enterobacter cloacae. In some embodiments, the Gram-negative bacterium is selected from the group consisting of Escherichia coli, Klebsiella pneumoniae, Enterobacter cloacae, and Enterobacter aerogenes. In some embodiments, the Gram-negative bacterium is selected from the group consisting of Escherichia coli, Klebsiella pneumoniae, Pseudomonas aeruginosa, and Acinetobacter baumannii.

In some embodiments, a peptide described herein binds to the lipid A portion of a lipopolysaccharide. In some embodiments, a peptide described herein has a lipopolysaccharide-binding affinity in terms of Kd of ≤100 μM as measured by biolayer interferometry. In some embodiments, a peptide described herein has a lipopolysaccharide-binding affinity in terms of Kd of S 10 μM as measured by biolayer interferometry. In some embodiments, a peptide described herein has a lipopolysaccharide-binding affinity in terms of Kd of ≤1 M as measured by biolayer interferometry. In some embodiments, a peptide described herein binds to a lipopolysaccharide selectively over a bacterial membrane phospholipid. In some embodiments, the bacterial membrane phospholipid is phosphatidylethanolamine, phosphatidylglycerol, or cardiolipin.

In some embodiments, a peptide described herein has an IC₅₀ of ≤10 μM against a Gram-negative bacterium, as measured by an in vitro bacterial growth assay. In some embodiments, a peptide described herein has an IC₅₀ of ≤1 μM against a Gram-negative bacterium, as measured by an in vitro bacterial growth assay. In some embodiments, a peptide described herein has an IC₅₀ of ≤100 nM against a Gram-negative bacterium, as measured by an in vitro bacterial growth assay. In some embodiments, the IC₅₀ is measured by an in vitro bacterial growth assay in LB or Mueller Hinton II cation-adjusted broth at 37° C. In some embodiments, a peptide described herein has an MIC of ≤500 μM against a Gram-negative bacterium, as measured by an in vitro bacterial growth assay. In some embodiments, a peptide described herein has an MIC of ≤50 μM against a Gram-negative bacterium, as measured by an in vitro bacterial growth assay. In some embodiments, a peptide described herein has an MIC of ≤15 μM against a Gram-negative bacterium, as measured by an in vitro bacterial growth assay. In some embodiments, the MIC is measured by an in vitro bacterial growth assay in LB or Mueller Hinton II cation-adjusted broth at 37° C.

In certain embodiments according to (or as applied to) any of the embodiments above, a peptide described herein that binds to a lipopolysaccharide is conjugated to a therapeutic agent (e.g. an antibiotic or an antiseptic). In certain embodiments according to (or as applied to) any of the embodiments above, a peptide described herein that binds to a lipopolysaccharide is conjugated to a label. In certain embodiments according to (or as applied to) any of the embodiments above, the label is a radioisotope, a fluorescent dye, or an enzyme.

### Affinity

In certain aspects, a peptide provided herein has a dissociation constant (Kd) of ≤1 mM, ≤100 μM, ≤10 μM, ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁺⁹ M to 10⁻¹³ M). In certain aspects, a peptide provided herein has a dissociation constant (Kd) of about 1 mM to about 100 μM, about 100 M to about 10 μM, about 10 μM to about 1 μM, about 1 μM to about 100 nM, about 100 nM to about 10 nM, about 10 nM to about 1 nM, about 1 nM to about 0.1 nM, about 0.1 nM to about 0.01 nM, or about 0.01 nM to about 0.001 nM. In one aspect, Kd is measured using an OCTET@ biolayer interferometry assay. In one aspect, Kd is measured using surface plasmon resonance assay, for example, an assay using a BIACORE®-2000 or a BIACORE®-3000 (BIAcore, Inc., Piscataway, N.J.). The equilibrium dissociation constant (Kd) is calculated as the ratio k_{off}/kₒₙ.

### Preparative Methods

Further provided herein is an isolated nucleic acid encoding a peptide described herein that binds to a lipopolysaccharide as provided herein. Also provided is an expression vector encoding the nucleic acid molecule as provided herein. Also provided is a cell comprising the expression vector of any one of the embodiments herein. Also provided is a method of producing a peptide described herein that binds to a lipopolysaccharide as provided herein, comprising culturing the cell as set forth herein, and recovering the peptide that binds to a lipopolysaccharide from the cell culture. Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5th edition, Garland Science, New York, N.Y., 2008; Voet, D. et al. “Fundamentals of Biochemistry: Life at the Molecular Level,” 3rd edition, John Wiley & Sons, Hoboken, N.J., 2008; Sambrook, J. et al., “Molecular Cloning: A Laboratory Manual.” 3rd edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4th edition, John Wiley & Sons, Somerset, N J, 2000; and the series “Methods in Enzymology,” Academic Press, San Diego, Calif.

Also provided is a method of producing a peptide described herein that binds to a lipopolysaccharide as provided herein, comprising chemically synthesizing the peptide that binds to a lipopolysaccharide. The peptides described herein (e.g., peptides that bind to a lipopolysaccharide) may be made and purified by methods known in the art, preferably by in vitro automated synthesis, for example using standard fluorenylmethoxycarbonyl (Fmoc)/tert-butyl (tBu) solid-phase methods known to those practiced in the arts (Chan, W. C., White, P. D., Eds. Fmoc Solid Phase Peptide Synthesis: A Practical Approach; Oxford University Press: New York, 2000.; Albericio, Fernando; Tulla-Puche, Judit; Kates, Steven A. Amino Acids, Peptides and Proteins in Organic Chemistry Volume 3, Pages 349-369, 2011). Furthermore, these peptides can be synthesized using D- or L-amino acids and selected non-natural or other modified amino acids, as is known in the art. The peptides can be stored in lyophilized form and dissolved in aqueous buffers or water prior to use. For the purposes of experimental use, the peptides can be dissolved in sterilized water or buffer. In addition, suitable buffers or diluents should be capable of solubilizing the active peptide, preferably at a suitable pH to prevent the peptide from precipitating out of solution too easily. In one embodiment, the peptides are tagged with detectable agents including, but not limited to, peptides, radioanalogs, products or compounds having distinctive absorption, fluorescence, or chemi-luminescence properties, such as rhodamine, fluorescein, green fluorescent protein (GFP) or semiconductor nanocrystal beads. Such peptides can, for example, be used as therapeutic and/or imagining agents.

### Assays

Peptides that bind to a lipopolysaccharide as provided herein may be identified, screened for, or characterized for their physical/chemical properties and/or biological activities by various assays known in the art. In one aspect, a peptide described herein (e.g. a peptide that binds to a lipopolysaccharide) is tested for its LPS binding activity, e.g., by known methods such as biolayer interferometry, surface plasmon resonance, etc. In one aspect, assays are provided for identifying peptides that bind to a lipopolysaccharide having biological activity. Biological activity may include, e.g., antibacterial activity. Peptides having such biological activity in vivo and/or in vitro are also provided. In certain aspects, a peptide described herein (e.g. a peptide that binds to a lipopolysaccharide) is tested for such biological activity.

In certain aspects, a peptide described herein (e.g. a peptide that binds to a lipopolysaccharide) has a 50% inhibition of bacterial growth concentration (IC₅₀) of ≤1 mM, ≤100 μM, ≤10 μM, ≤1 μM, ≤100 nM, ≤10 nM, or ≤1 nM. In certain aspects, a peptide provided herein has a 50% inhibition of bacterial growth concentration (IC₅₀) of about 1 mM to about 100 μM, about 100 μM to about 10 μM, about 10 μM to about 1 μM, about 1 μM to about 100 nM, about 100 nM to about 10 nM, or about 10 nM to about 1 nM. In one aspect, IC₅₀ is measured using an in vitro bacterial cell growth inhibition assay.

In certain aspects, a peptide provided herein has a lowest concentration that prevents visible bacterial growth (MIC) of ≤5 mM, ≤500 μM, ≤50 μM, ≤25 μM, ≤15 μM, ≤5 μM, ≤500 nM, ≤50 nM, or ≤5 nM. In certain aspects, a peptide provided herein has a lowest concentration that prevents visible bacterial growth (MIC) of about 5 mM to about 500 μM, about 500 μM to about 50 μM, about 50 μM to about 5 μM, about 5 μM to about 500 nM, about 500 nM to about 50 nM, or about 50 nM to about 5 nM. In one aspect, MIC is measured using an in vitro bacterial cell growth inhibition assay.

### Methods and Compositions

In certain aspects, any of the peptides that bind to a lipopolysaccharide provided herein is useful for detecting the presence of lipopolysaccharide in a biological sample. The term “detecting” as used herein encompasses quantitative or qualitative detection.

In one aspect, a peptide described herein (e.g. that binds to a lipopolysaccharide) can be used in a method of diagnosis or detection. In a further aspect, a method of detecting the presence of lipopolysaccharide in a biological sample is provided. In certain aspects, the method comprises contacting the biological sample with a peptide that binds to a lipopolysaccharide as described herein under conditions permissive for binding of the peptide that binds to a lipopolysaccharide to lipopolysaccharide, and detecting whether a complex is formed between the peptide that binds to a lipopolysaccharide and lipopolysaccharide. Such method may be an in vitro or in vivo method. In one aspect, a peptide that binds to a lipopolysaccharide is used to select subjects eligible for therapy with a peptide that binds to a lipopolysaccharide, e.g., where lipopolysaccharide is a biomarker for selection of patients.

Exemplary disorders that may be diagnosed using a peptide described herein (e.g. a peptide that binds to a lipopolysaccharide) include Gram-negative bacterial infections, such as, Escherichia coli. Klebsiella spp. (including but not limited to Klebsiella pneumoniae, Klebsiella oxytoca, and Klebsiella granulomatis), Pseudomonas spp. (including by not limited to Pseudomonas aeruginosa), Acinetobacter (including but not limited to Acinetobacter baumannii), Enterobacter spp. (including but not limited to Enterobacter aerogenes and Enterobacter cloacae), Bordatella spp., Burkholderia spp., Stenotrophomonas maltophiha, Bacteroides spp., Campylobacter spp., Francisella tularensis, Helicobacter pylori, Legionella spp., and Vibrio spp.

In certain aspects, labeled peptides that bind to a lipopolysaccharide are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, f-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Compositions

In a further aspect, provided are pharmaceutical compositions and medicaments comprising any of the peptides provided herein, e.g., for use as described herein. In one aspect, a pharmaceutical composition comprises any of the peptides provided herein and a pharmaceutically acceptable carrier. In another aspect, a pharmaceutical composition comprises any of the peptides provided herein and at least one additional therapeutic agent, e.g., as described below. Also provided are methods of using the peptides described herein (e.g. peptides that bind to a lipopolysaccharide) to prepare such pharmaceutical compositions.

Pharmaceutical compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

A peptide described herein (e.g. a peptide that binds to a lipopolysaccharide) (and any additional therapeutic agent) may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The peptides described herein (e.g. peptides that bind to a lipopolysaccharide) may be administered in any convenient administrative form, e.g., tablets, powders, capsules, solutions, dispersions, suspensions, syrups, sprays, suppositories, gels, emulsions, patches, etc. Such compositions may comprise components conventional in pharmaceutical preparations, e.g., diluents, carriers, pH modifiers, preservatives, solubilizers, stabilizers, wetting agents, emulsifiers, sweeteners, colorants, flavorants, salts for varying the osmotic pressure, buffers, masking agents, antioxidants, and further active agents. They can also comprise still other therapeutically valuable substances.

A typical formulation is prepared by mixing a peptide described herein and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a peptide described herein or pharmaceutical composition thereof) or aid in the manufacturing of the pharmaceutical product (i.e., medicament). The dosage at which peptides described herein (e.g. peptides that bind to a lipopolysaccharide) can be administered can vary within wide limits and will, of course, be fitted to the individual requirements in each particular case. In general, in the case of oral administration a daily dosage of about 0.01 to 1000 mg per person of a peptide described herein (e.g. a peptide that binds to a lipopolysaccharide) should be appropriate, although the above upper limit can also be exceeded when necessary.

An example of a suitable oral dosage form is a tablet comprising about 100 mg to 500 mg of a peptide described herein described herein compounded with about 30 to 90 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment.

An example of an aerosol formulation can be prepared by dissolving the peptide, for example 10 to 100 mg, described herein in a suitable buffer solution, e.g. a phosphate buffer, adding a tonicifier, e.g. a salt such as sodium chloride, if desired. The solution may be filtered, e.g., using a 0.2 μm filter, to remove impurities and contaminants.

Pharmaceutical compositions of a peptide described herein may be prepared by mixing such peptide having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydophilic polymers such as polyvinylpyrrolidone; amino acids such as glycine, glutamine, asparagine, histidine, arginine, or lysine; monosaccharides, disaccharides, and other carbohydrates including glucose, mannose, sucrose, mannitol, trehalose, sorbitol, or dextrins; chelating agents such as EDTA; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The pharmaceutical composition herein may also contain more than one active ingredient as necessary for the particular indication being treated, preferably those with complementary activities that do not adversely affect each other. For example, it may be desirable to further provide one or more antibiotics, and/or one or more antiseptics. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the peptide, which matrices are in the form of shaped articles, e.g., films, or microcapsules.

The pharmaceutical compositions to be used for in vivo administration may be sterile. Sterility may be readily accomplished, e.g., by filtration through sterile filtration membranes.

### Therapeutic Methods and Routes of Administration

Any of the peptides that bind to a lipopolysaccharide provided herein may be used in therapeutic methods.

Provided herein are peptides (e.g. a peptide that binds to a lipopolysaccharide) for use as a medicament. In one embodiment, is a peptide described herein (e.g. a peptide that binds to a lipopolysaccharide) for use in a method of treating an individual having a bacterial infection, the method comprising administering to the individual an effective amount of the peptide. The method can further comprise administering to the individual an effective amount of at least one additional therapeutic agent (e.g., one, two, three, four, five, or six additional therapeutic agents), e.g., as described below. An “individual” according to any of the above aspects is preferably a human. In still another embodiment provided herein is a peptide as provided herein for use in treating a bacterial infection, where the bacterial infection is optionally a gram-negative bacteria infection as set forth herein.

In a further aspect, the use of a peptide described herein (e.g. a peptide that binds to a lipopolysaccharide) in the manufacture or preparation of a medicament is provided. In one aspect, the medicament is for treatment of a bacterial infection, the method comprising administering to an individual having a bacterial infection an effective amount of the medicament. In one such aspect, the method further comprises administering to the individual an effective amount of at least one additional therapeutic agent, e.g., as described below. In one embodiment, an individual is a human.

Further provided herein is a method for treating a bacterial infection, the method comprises administering to an individual having such bacterial infection an effective amount of a peptide described herein (e.g. a peptide that binds to a lipopolysaccharide). In one embodiment, the method further comprises administering to the individual an effective amount of at least one additional therapeutic agent, as described below. In one embodiment, an individual is a human.

Further provided herein are pharmaceutical compositions comprising any of the peptides described herein (e.g. a peptide that binds to a lipopolysaccharide), e.g., for a use provided herein and a pharmaceutically acceptable carrier. In one embodiment, a pharmaceutical composition comprises any of the peptides that bind to a lipopolysaccharide provided herein and at least one additional therapeutic agent, e.g., as described below.

Peptides described herein (e.g. peptides that bind to a lipopolysaccharide) can be administered alone or used in a combination therapy. For instance, the combination therapy includes administering a peptide described herein (e.g. a peptide that binds to a lipopolysaccharide) and administering at least one additional therapeutic agent (e.g. one, two, three, four, five, or six additional therapeutic agents). In certain aspects, the combination therapy comprises administering a peptide described herein (e.g. a peptide that binds to a lipopolysaccharide) and administering at least one additional therapeutic agent, such as antibiotics and antiseptics.

Such combination therapies noted above encompass combined administration (where two or more therapeutic agents are included in the same or separate pharmaceutical compositions), and separate administration, in which case, administration of a peptide described herein described herein can occur prior to, simultaneously, and/or following, administration of the additional therapeutic agent or agents. In one aspect, administration of a peptide described herein that binds to a lipopolysaccharide and administration of an additional therapeutic agent occur within about one month, or within about one, two or three weeks, or within about one, two, three, four, five, or six days, of each other. In one aspect, a peptide described herein and additional therapeutic agent are administered to the patient on Day 1 of the treatment.

Peptides described herein (e.g. peptides that bind to a lipopolysaccharide) can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. A peptide described herein need not be, but is optionally formulated with one or more agents currently used to prevent or treat the disorder in question. The effective amount of such other agents depends on the amount of peptide present in the pharmaceutical composition, the type of disorder or treatment, and other factors discussed above.

For the prevention or treatment of disease, the appropriate dosage of a peptide described herein (e.g. a peptide that binds to a lipopolysaccharide) (when used alone or in combination with one or more other additional therapeutic agents) will depend on the type of disease to be treated, the type of peptide, the severity and course of the disease, whether a peptide described herein is administered for preventive or therapeutic purposes, previous therapy, the patient's clinical history and response to the peptide, and the discretion of the attending physician. The peptide is suitably administered to the patient at one time or over a series of treatments. Depending on the type and severity of the disease, about 1 μg/kg to 15 mg/kg (e.g., 0.1 mg/kg-10 mg/kg) of peptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of a peptide described herein would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the peptide). An initial higher loading dose, followed by one or more lower doses may be administered. The progress of this therapy is easily monitored by conventional techniques and assays.

### Bacterial Infections

In certain embodiments described herein, a bacterial infection refers to respiratory tract infection (RTI), community-acquired pneumonia (CAP). In certain embodiments, a bacterial infection refers to nosocomial pneumonia (NP). In certain embodiments, a bacterial infection refers to hospital-acquired pneumonia (HAP). In certain embodiments, a bacterial infection refers to ventilator associated pneumonia (VAP). In certain embodiments, a bacterial infection refers to bacteremia. In certain embodiments, a bacterial infection refers to a bloodstream infection (BSI). In certain embodiments, a bacterial infection refers to central line associated bloodstream infection. In certain embodiments, a bacterial infection refers to intra-abdominal infection (IAI). In certain embodiments, a bacterial infection refers to complicated intra-abdominal infection (cIAI). In certain embodiments, a bacterial infection refers to skin and soft tissue infection (SSTI). In certain embodiments, a bacterial infection refers to complicated skin and soft tissue infection (cSSTI). In certain embodiments, a bacterial infection refers to surgical site infection (SSI). In certain embodiments, a bacterial infection refers to complicated surgical site infection (cSSI). In certain embodiments, skin and soft tissue infection is cellulitis. In certain embodiments, a bacterial infection refers to skin and skin structure infection (SSSI). In certain embodiments, a bacterial infection refers to complicated skin and skin structure infection (cSSSI). In certain embodiments, a bacterial infection refers to osteomyelitis. In certain embodiments, a bacterial infection refers to prosthetic joint infection. In certain embodiments, a bacterial infection refers to a urinary tract infection (UTI). In certain embodiments, a bacterial infection refers to a complicated urinary tract infection (cUTI). In certain embodiments, a bacterial infection refers to post-operative infection.

Further provided herein, is a method of treating a bacterial infection in an individual, a method of preventing a bacterial infection in an individual, or a method of reducing the risk of acquiring a bacterial infection in an individual, the method comprising administering to the individual a therapeutically effective amount of an antibacterial peptide described herein, thereby treating the bacterial infection, preventing the bacterial infection, or reducing the risk of acquiring the bacterial infection. In one embodiment, is a method of treating a bacterial infection in an individual, the method comprising administering to the individual a therapeutically effective amount of an antibacterial peptide described herein

In some embodiments, is a method for treating, preventing, or reducing the risk of acquiring a bacterial infection, wherein the bacterial infection is selected from the group consisting of a RTI, a lung infection, an upper respiratory tract infection, a lower respiratory tract infection, a nasopharyngeal infection, a UTI, a cUTI, pneumonia, NP, CAP, HAP, VAP, bacteremia, a BSI, central line associated bloodstream infection, IAI, cIAI, SSTI, cSSTI, SSI, cSSI, SSSI, cSSSI, osteomyelitis, prosthetic joint infection, and post-operative infection, the method comprising administering to the individual a therapeutically effective amount of an antibacterial peptide described herein described herein, thereby treating, preventing, or reducing the risk of acquiring the bacterial infection. In one embodiment, the method is a method of reducing the risk of acquiring a bacterial infection in an individual described herein by administering a therapeutically effective amount of a peptide described herein.

In some embodiments, the bacterial infection occurs at the site of a foreign device such as but not limited to a shunt or intraventricular catheter. In certain embodiments, the bacterial infection is a Gram-negative bacterial infection.

In some embodiments, is a method for treating, preventing, or reducing the risk of acquiring a RTI in an individual in need thereof, the method comprising administering to the individual a therapeutically effective amount of an antibacterial peptide described herein, thereby treating, preventing, or reducing the risk of acquiring the respiratory tract infection. In some embodiments, is a method for treating, preventing, or reducing the risk of acquiring a lung infection in an individual in need thereof, the method comprising administering to the individual a therapeutically effective amount of an antibacterial peptide described herein, thereby treating, preventing, or reducing the risk of acquiring the lung infection. In some embodiments, is a method for treating, preventing, or reducing the risk of acquiring an upper respiratory tract infection in an individual in need thereof, the method comprising administering to the individual a therapeutically effective amount of an antibacterial peptide described herein, thereby treating, preventing, or reducing the risk of acquiring the upper respiratory tract infection. In some embodiments, is a method for treating, preventing, or reducing the risk of acquiring a lower respiratory tract infection in an individual in need thereof, the method comprising administering to the individual a therapeutically effective amount of an antibacterial peptide described herein, thereby treating, preventing, or reducing the risk of acquiring the lower respiratory tract infection. In some embodiments, is a method for treating, preventing, or reducing the risk of acquiring a nasopharyngeal infection in an individual in need thereof, the method comprising administering to the individual a therapeutically effective amount of an antibacterial peptide described herein, thereby treating, preventing, or reducing the risk of acquiring the nasopharyngeal infection.

In some embodiments, is a method for treating, preventing, or reducing the risk of acquiring pneumonia in an individual in need thereof, the method comprising administering to the individual a therapeutically effective amount of an antibacterial peptide described herein, thereby treating, preventing, or reducing the risk of acquiring pneumonia. In some embodiments, is a method for treating, preventing, or reducing the risk of acquiring CAP in an individual in need thereof, the method comprising administering to the individual a therapeutically effective amount of an antibacterial peptide described herein, thereby treating, preventing, or reducing the risk of acquiring community-acquired pneumonia. In some embodiments, is a method for treating, preventing, or reducing the risk of acquiring nosocomial pneumonia in an individual in need thereof, the method comprising administering to the individual a therapeutically effective amount of an antibacterial peptide described herein, thereby treating, preventing, or reducing the risk of acquiring nosocomial pneumonia. In some embodiments, is a method for treating, preventing, or reducing the risk of acquiring HAP in an individual in need thereof, the method comprising administering to the individual a therapeutically effective amount of an antibacterial peptide described herein, thereby treating, preventing, or reducing the risk of acquiring hospital-acquired pneumonia. In some embodiments, is a method for treating, preventing, or reducing the risk of acquiring ventilator-associated pneumonia (VAP) in an individual in need thereof, the method comprising administering to the individual a therapeutically effective amount of an antibacterial peptide described herein, thereby treating, preventing, or reducing the risk of acquiring ventilator-associated pneumonia.

In some embodiments, is a method for treating, preventing, or reducing the risk of acquiring a urinary tract infection in an individual in need thereof, the method comprising administering to the individual a therapeutically effective amount of an antibacterial peptide described herein, thereby treating, preventing, or reducing the risk of acquiring the urinary tract infection. In certain embodiments, is a method for treating, preventing, or reducing the risk of acquiring a urinary tract infection in an individual in need thereof, wherein the urinary tract infection is a bacterial infection of the bladder (cystitis), urethra (urethritis), kidney (pyelonephritis), or ureter, the method comprising administering to the individual a therapeutically effective amount of an antibacterial peptide described herein, thereby treating, preventing, or reducing the risk of acquiring a bacterial infection of the bladder, urethra, kidney, or ureter. In some embodiments, is a method for treating, preventing, or reducing the risk of acquiring bacteremia in an individual in need thereof, the method comprising administering to the individual a therapeutically effective amount of an antibacterial peptide described herein, thereby treating, preventing, or reducing the risk of acquiring bacteremia. In some embodiments, is a method for treating, preventing, or reducing the risk of acquiring a blood stream infection in an individual in need thereof, the method comprising administering to the individual a therapeutically effective amount of an antibacterial peptide described herein, thereby treating, preventing, or reducing the risk of acquiring the blood stream infection. In some embodiments, is a method for treating, preventing, or reducing the risk of acquiring a central line associated blood stream infection in an individual in need thereof, the method comprising administering to the individual a therapeutically effective amount of an antibacterial peptide described herein, thereby treating, preventing, or reducing the risk of acquiring the central line associated blood stream infection.

In some embodiments, is a method for treating, preventing, or reducing the risk of a bacterial infection at the site of a device implant (e.g., shunt or intraventricular catheter) in an individual in need thereof, thereby treating, preventing, or reducing the risk of the bacterial infection at the site of the device implant.

In some embodiments, is a method for treating, preventing, or reducing the risk of acquiring an intra-abdominal infection in an individual in need thereof, the method comprising administering to the individual a therapeutically effective amount of an antibacterial peptide described herein, thereby treating, preventing, or reducing the risk of acquiring the intra-abdominal infection. In some embodiments, is a method for treating, preventing, or reducing the risk of acquiring a skin and soft tissue infection in an individual in need thereof, the method comprising administering to the individual a therapeutically effective amount of an antibacterial peptide described herein, thereby treating, preventing, or reducing the risk of acquiring the skin and soft tissue infection. In some embodiments, is a method for treating, preventing, or reducing the risk of acquiring a complicated skin and soft tissue infection in an individual in need thereof, the method comprising administering to the individual a therapeutically effective amount of an antibacterial peptide described herein, thereby treating, preventing, or reducing the risk of acquiring the complicated skin and soft infection. In some embodiments, is a method for treating, preventing, or reducing the risk of acquiring a skin and skin structure infection in an individual in need thereof, the method comprising administering to the individual a therapeutically effective amount of an antibacterial peptide described herein, thereby treating, preventing, or reducing the risk of acquiring the skin and skin structure infection. In some embodiments, is a method for treating, preventing, or reducing the risk of acquiring a complicated skin and skin structure infection in an individual in need thereof, the method comprising administering to the individual a therapeutically effective amount of an antibacterial peptide described herein, thereby treating, preventing, or reducing the risk of acquiring the complicated skin and skin structure infection.

In some embodiments, is a method for treating, preventing, or reducing the risk of acquiring a bone infection in an individual in need thereof, the method comprising administering to the individual a therapeutically effective amount of an antibacterial peptide described herein, thereby treating, preventing, or reducing the risk of acquiring the bone infection. In some embodiments, is a method for treating, preventing, or reducing the risk of acquiring osteomyelitis in an individual in need thereof, the method comprising administering to the individual a therapeutically effective amount of an antibacterial peptide described herein, thereby treating, preventing, or reducing the risk of acquiring osteomyelitis. In some embodiments, is a method for treating, preventing, or reducing the risk of acquiring a prosthetic joint infection in an individual in need thereof, the method comprising administering to the individual a therapeutically effective amount of an antibacterial peptide described herein, thereby treating, preventing, or reducing the risk of acquiring the prosthetic joint infection. In some embodiments, is a method for treating, preventing, or reducing the risk of acquiring a post-operative infection in an individual in need thereof, the method comprising administering to the individual a therapeutically effective amount of an antibacterial peptide described herein, thereby treating, preventing, or reducing the risk of acquiring the post-operative infection.

### Gram-Negative Bacteria

In certain embodiments, a bacterial infection refers to an infection caused by Gram-negative bacteria (e.g., Gram-negative bacterial infection). Gram-negative bacteria include, but are not limited to Escherichia coli, Klebsiella spp. (including but not limited to Klebsiella pneumoniae, Klebsiella oxytoca, and Klebsiella granulomatis), Pseudomonas spp. (including by not limited to Pseudomonas aeruginosa), Acinetobacter (including but not limited to Acinetobacter baumannii), Enterobacter spp. (including but not limited to Enterobacter aerogenes and Enterobacter cloacae), Bordatella spp., Burkholderia spp., Stenotrophomonas maltophilia, Bacteroides spp., Campylobacter spp., Francisella tularensis, Helicobacter pylori. Legionella spp., and Vibrio spp. In another embodiment, a bacterial infection refers to an infection related to gram-negative bacteria as described herein.

Combination with Standard of Care

In certain embodiments, an antibacterial peptide described herein can be administered in combination with one or more additional therapeutic agents for treating, preventing, or reducing the risk of a bacterial infection. In some embodiments, therapeutic agents administered in combination are administered as part of a single composition (i.e., a single composition comprising the antibacterial peptide described herein and one or more additional therapeutic agents). In some embodiments, such therapeutic agents are administered separately from the antibacterial peptide described herein, e.g., as one or more separate compositions. In some embodiments, the additional therapeutic agent is, e.g., an antibiotic. In some embodiments, the additional therapeutic agent is, e.g., an antiseptic. Thus, in some embodiments, the antibacterial peptide described herein is administered in combination with an antibiotic. In some embodiments, the antibacterial peptide described herein is administered in combination with an antiseptic. In some embodiments, the antibacterial peptide described herein is administered in combination with one or more antibiotic(s) and one or more antiseptic(s). When administered separately from the antibacterial peptide described herein, the one or more additional therapeutic agent(s) may be administered simultaneously or sequentially, and in either case each additional therapeutic agent is said to be co-administered, i.e., administered in combination. A person skilled in the art will know how to administer, for example, one or more antibiotics, one or more antiseptics, and/or the antibacterial peptide described herein in combination, e.g., for treating, preventing, or reducing the risk of bacterial infection.

Combination therapy (i.e., administration of an antibacterial peptide described herein in combination with one or more additional therapeutic agents) may result in a synergistic effect, i.e., the agents acting together may create an effect greater than that predicted by knowing only the separate effects of the individual agents. Such a synergistic effect might be particularly advantageous if lower amounts of the antibacterial peptide described herein and/or one or more of the additional therapeutic agents may then be used. Thus, in certain embodiments, possible side-effects of the antibacterial peptide described herein described herein, and/or of other antibiotic(s) and/or antiseptic(s), e.g., antibiotic(s) and/or antiseptic(s) as disclosed herein, may be diminished or avoided.

Further provided herein are antibacterial peptides described herein linked, for example, by formation of a conjugate, to one or more additional therapeutic agents, e.g., an antibiotic(s) and/or antiseptic(s) (e.g., as described herein). In such embodiment, the additional therapeutic agent is considered to be administered in combination with the antibacterial peptide described herein described herein.

According to current standards of care in the setting of Gram-negative bacterial infections, prompt treatment initiation is critical to ensure clinical success; delayed treatment can be associated with less-favorable clinical outcomes. As Gram-negative pathogen confirmation can take 12-48 hours, and full antibiotic susceptibility testing can take 24-72 hours, individuals with suspected Gram-negative bacterial infections are often treated with empiric therapy.

Empiric therapy refers to treatment of the individual on the basis of symptoms, professional experience, local epidemiology, site and severity of infection, as well as patient risk factors in consideration of drug-resistant pathogens. Empiric therapy for bacterial infection is typically used prior to obtaining laboratory test results confirming the bacterial infection type and antibiotic susceptibility. Empiric therapy is most often used for bacterial infection when antibiotics are given to the individual before the specific bacterium causing the infection is known or identified, such as by a confirmatory laboratory test. Empiric treatment of a bacterial infection is typically with a broad-spectrum antibiotic, often with a broad-spectrum antibiotic treatment regimen effective at treating both Gram-positive and Gram-negative bacteria. Once laboratory results with pathogen confirmation are available, the choice of treatment may be modified based on the laboratory results. Treatment guidelines encourage physicians to de-escalate from broad-spectrum antibiotic agents to therapies with narrow spectra upon pathogen confirmation; however, such practice in not always followed, such as, for example, in situations where the patient is improving, wherein physicians may opt to keep the patient on the same initial empiric therapy. Furthermore, a person of skill in the art will appreciate that the treatment regimen, e.g., selection of the therapeutic agent, dose, combination and/or order of sequential therapeutic agent use can vary depending on the site and/or source of the bacterial infection. For further guidance, see Gilbert, David N., et al. Sanford Guide to Antimicrobial Therapy 2016. Sperryville, Va.: © 1969-2016 by Antimicrobial Therapy, Inc. 2016.

Thus, in certain embodiments, an antibacterial peptide described herein may be administered in combination with one or more therapeutic agents as part of an empiric therapy and/or a current standard of care, for treating, preventing, or reducing the risk of Gram-negative bacterial infection. For example, and without limitation, an antibacterial peptide described herein may be administered in combination with one or more antibiotics and/or one or more antiseptics for treating, preventing, or reducing the risk of a bacterial infection, e.g., a Gram-negative bacterial infection. Further, an antibacterial peptide may be formulated as a pharmaceutical formulation further comprising one or more antibiotic(s) and/or antiseptic(s), e.g., as exemplified herein.

By way of example and without limitation, one or more antibiotics that may be administered in combination with the antibacterial peptide described herein described herein, include, for example, aminoglycoside-derived antibiotics such as, e.g., streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, plazomicin, sisomicin, isepamicin, verdamicin, astromicin, rhodostreptomycin, apramycin; steroid-derived antibiotics such as, e.g., fusidic acid, or sodium fusidate; glycopeptide-derived antibiotics such as, e.g., vancomycin, oritavancin, telavancin, teicoplanin, dalbavancin, ramoplanin, bleomycin, decaplanin; tetracycline-derived antibiotics such as, e.g., tetracycline, doxycycline, chlortetracycline, clomocycline, demeclocycline, lymecycline, meclocycline, metacycline, minocycline, oxytetracycline, penimepicycline, rolitetracycline, tigccycline or eravacycline; amphenicol-derived antibiotics such as, e.g., chloramphenicol, azidamfenicol, thiamphenicol, or florfenicol; macrolide-derived antibiotics such as, e.g., erythromycin, azithromycin, spiramycin, midecamycin, oleandomycin, roxithrvmycin, josamycin, troleandomycin, clarithromycin, miocamycin, rokitamycin, dirithromycin, flurithromycin, telithromycin, cethromycin, solithromycin, tulathromycin, carbomycin A, kitasamycin, midecamicine, midecamicine acetate, fosfomycin, tylosin (tylocine); or ketolide-derived antibiotics such as, e.g., telithromycin, cethromycin; lincosamide-derived antibiotics such as, e.g., clindamycin, lincomycin, pirlimycin; streptogramin-derived antibiotics such as, e.g., pristinamycin, quinupristin/dalfopristin, virginiamycin; oxazolidinone-derived antibiotics such as, e.g., linezolid, tedizolid, eperezolid, posizolid, radezolid, ranbenzolid, sutezolid or cycloserine; peptidyl transferases such as, e.g., chloramphenicol, azidamfenicol, thiamphenicol, florfenicol, retapamulin, tiamulin, valnemulin; beta-lactam-derived antibiotics such as, e.g., amoxicillin, ampicillin, pivampicillin, hetacillin, bacampicillin, metampicillin, talampicillin, epicillin, carbcnicillin, carindacillin, ticarcillin, temocillin, azlocillin, piperacillin, mezlocillin, mecillinam, pivmecillinam, sulbenicillin, benzylpenicillin, azidocillin, penamecillin, clometocillin, benzathine benzylpenicillin, procaine benzylpenicillin, phenoxymethylpenicillin, propicillin, benzathine, phenoxymethylpenicillin, pheneticillin, oxacillin, cloxacillin, dicloxacillin, flucloxacillin, meticillin, nafcillin, faropenem, biapenem, doripenem, ertapenem, imipenem, meropenem, panipenem, cefacetrile, cefadroxil, cefalexin, cefaloglycin, cefalonium, cefaloridine, cefalotin, cefapirin, cefatrizine, cefazedone, cefazaflur, cefazolin, cefradine, cefroxadine, ceftezole, cefaclor, cefamandole, cefminox, cefonicid, ceforanide, cefotiam, cefprozil, cefbuperazone, cefuroxime, cefuzonam, cefoxitin, cefotetan, cefinetazole, loracarbef, cefcapene, cefdaloxime, cefdinir, cefditoren, cefetamet, cefixime, cefmenoxime, cefodizime, cefoperazone, cefotaxime, cefpimizole, cefpiramide, cefpodoxime, cefsulodin, ceftazidime, ceftaroline, cefteram, ceftibuten, ceftiolene, ceftizoxime, ceftriaxone, flomoxef, latamoxef, cefepime, cefozopran, cefpirome, ccfquinome, ceftobiprole, aztreonam, tigemonam, sulbactam, tazobactam, clavulanate, clavulanic acid, ampicillin/sulbactam, sultamicillin, piperacillin/tazobactam, co-amoxiclav, amoxicillin/clavulanic acid, ceftazidime/avibactam, ceftolozane/tazobactam, piperacillin/tazobactam, meropenem/RPX-7009, imipenem/cilastatin/relebactam, amoxicillin/clavulanate, or imipenem/cilastatin; sulfonamide-derived antibiotics such as, e.g., acetazolamide, benzolamide, bumetanide, celecoxib, chlorthalidone, clopamide, dichlorphenamide, dorzolamide, ethoxzolamide, furosemide, hydrochlorothiazide, indapamide, mafenide, mefruside, metolazone, probenecid, sulfacetamide, sulfadiazine, sulfadimethoxine, sulfadoxine, sulfanilamides, sulfamethoxazole, sulfamethoxypyridazine, sulfasalazine, sultiame, sumatriptan, xipamide, zonisamide, sulfaisodimidine, sulfamethizole, sulfadimidine, sulfapyridine, sulfafurazole, sulfathiazole, sulfathiourea, sulfamoxole, sulfadimethoxine, sulfalene, sulfametomidine, sulfametoxydiazine, sulfaperin, sulfamerazine, sulfaphenazole, or sulfamazone; quinolone-derived antibiotics such as, e.g., cinoxacin, flumequine, nalidixic acid, oxolinic acid, pipemidic acid, piromidic acid, rosoxacin, ciprofloxacin, enoxacin, fleroxacin, lomefloxacin, nadifloxacin, ofloxacin, norfloxacin, pefloxacin, rufloxacin, balofloxacin, grepafloxacin, levofloxacin, pazufloxacin, sparfloxacin, temafloxacin, tosufloxacin, besifloxacin, clinafloxacin, garenoxacin, gemifloxacin, moxifloxacin, gatifloxacin, sitafloxacin, trovafloxacin, alatrofloxacin, prulifloxacin, danofloxacin, difloxacin, enrofloxacin, ibafloxacin, marbofloxacin, orbifloxacin, pradofloxacin, sarafloxacin, ecinofloxacin, or delafloxacin; imidazole-derived antibiotics such as, e.g., metronidazole; nitrofuran-derived antibiotics such as, e.g., nitrofurantoin, or nifurtoinol; aminocoumarin-derived antibiotics such as, e.g., novobiocin, clorobiocin, or coumermycin A 1; ansamycin-derived antibiotics, including rifamycin-derived antibiotics such as, e.g., rifampicin (rifampin), rifabutin, rifapentine, or rifaximin; and also further antibiotics such as, e.g., bacitracin, colistin, polymyxin B, daptomycin, xibornol, clofoctol, methenamine, mandelic acid, nitroxoline, mupirocin, trimethoprim, brodimoprim, iclaprim, tetroxoprim, or sulfametrole; pleuromutilins, e.g., lefamulin; without being limited thereto.

Furthermore, the one or more antiseptics can include, for example, acridine-derived antiseptics such as, e.g., ethacridine lactate, aminoacridine, or euflavine; amidine-derived or biguanide-derived antiseptics such as, e.g., dibrompropamidine, chlorhexidine, propamidine, hexamidine, or polihexanide; phenol-derived antiseptics such as, e.g., phenol, hexachlorophene, policresulen, triclosan, chloroxylenol, or biphenylol; nitrofuran-derived antiseptics such as, e.g., nitrofurazone; iodine-based antiseptics such as, e.g., iodine/octylphenoxypolyglycolether, povidone-iodine, or diiodohydroxypropane; quinoline-derived antiseptics such as, e.g., dequalinium, chlorquinaldol, oxyquinoline, or clioquinol; quatemary ammonium-derived antiseptics such as, e.g., benzalkonium, cetrimonium, cetylpyridinium, cetrimide, benzoxonium chloride, or didecyldimethylammonium chloride; mercurial antiseptics such as, e.g., mercuric amidochloride, phenylmercuric borate, mercuric chloride, mercurochrome, thiomersal, or mercuric iodide; silver-based antiseptics such as, e.g., silver nitrate; alcoholic antiseptics such as, e.g., propanol (including isopropanol), or ethanol; and also further antiseptics such as, e.g., potassium permanganate, sodium hypochlorite, hydrogen peroxide, cosin, tosylchloramide sodium, dichlorobenzyl alcohol, ambazone, benzethonium, myristyl-benzalkonium, hexylresorcinol, or acriflavinium chloride; without being limited thereto.

In certain embodiments, the administration of the antibacterial peptide described herein in combination with one or more therapeutic agents can be for treating, preventing, or reducing the risk of acquiring a bacterial infection, wherein the bacterial infection is selected from the group consisting of a RT), a lung infection, an upper respiratory tract infection, a lower respiratory tract infection, a nasopharyngeal infection, a UTI, pneumonia, NP, CAP, HAP, VAP, bacteremia, a BSI, central line associated bloodstream infection, IAI, cIAI, SST), cSSTI, SSI, cSSI, SSSI, cSSSI, osteomyelitis, prosthetic joint infection, a post-operative infection, and, a bacterial infection at a site of a foreign device such as but not limited to a shunt or intraventricular catheter.

In certain embodiments, an antibacterial peptide described herein may be administered in combination with a standard of care for treating, preventing, or reducing the risk of a Gram-negative infection.

Antibiotic therapy or treatment for Gram-negative infections target various mechanisms of action in eliciting their anti-microbial effects. These include cell-wall synthesis inhibitors (e.g., penicillins, cephalosporins, carbapenems, beta-lactams, and monobactams); protein synthesis inhibitors (e.g., aminoglycosides, tetracyclines and tetracycline derivatives); nucleic acid synthesis inhibitors (e.g., quinolones, nitroimidazoles, diaminopyrimidies); and cell membrane structural inhibitors (e.g., polymyxins).

Treatment for Gram-negative bacterial infections include Penicillins (e.g., Amoxicillin, Ampicillin, Piperacillin); Beta-lactam/beta-lactamase inhibitors and combinations (e.g., Amoxicillin/clavulanate, Ampicillin/sulbactam, Ceftazidime/avibactam, Ceftolozane/tazobactam, Piperacillin/tazobactam, Meropenem/RPX-7009, Imipenem/cilastatin/relebactam); Cephalosporins (first generation Cephalosporins such as, e.g., Cefazolin; second generation Cephalosporins such as, e.g., Cefuroxime; third generation Cephalosporins such as, e.g., Ceftriaxone, Ceftazidime, and Cefotaxime; fourth generation Cephalosporins such as, e.g., Cefepime; fifth generation Cephalosporins such as, e.g., Ceftaroline and Ceftobiprole); Carbepenems (e.g., Doripenem, Imipenem/cilastatin, Meropenem, Ertapenem); monobactams (e.g., Aztreonam); Aminoglycosides (e.g., Gentamicin, Tobramycin, Amikacin, Arbekacin, Plazomicin); Tetracyclines/glycylcyclines (e.g., Tigecycline, Doxycycline, Minocycline, Eravacycline); Lincosamides (e.g., Clindamycin); Quinolones (e.g., Ciprofloxacin, Levofloxacin, Moxifloxacin, Garenoxacin, Ofloxacin, Sitafloxacin, Delafloxacin); Diaminopyrimidines (e.g., Trimethoprim/sulfamethoxazole); Phosphomycins (e.g., Fosfomycin); Polymyxins (e.g., Colistin, Polymyxin B); Dihydrofolate reductase inhibitors (e.g., Trimethoprim, Trimethoprim/sulfiethoxazole); and Nitroimidazoles (e.g., Metronidazole).

Generally, five drug classes are most often used for the treatment of Gram-negative bacterial infections: cephalosporins, quinolones, beta-lactam/beta-lactamase inhibitor combinations, aminoglycosides, and carbapenems. In particular, three therapies (piperacillin/tazobactam, imipenem/cilastatin, and meropenem make up nearly half of the total patient share for treatment of Gram-negative infections in hospital settings. Empiric treatment for a suspected Gram-negative bacterial infection is most common for (but not limited to) individuals with urinary tract infections, complicated intra-abdominal infections, and nosocomial pneumonia infections. Antibiotics from the cephalosporin class and the beta-lactam/beta-lactamase inhibitor class are often the empiric first-line treatments.

In other aspects, is methods of treating a bacterial infection in an individual, methods of preventing a bacterial infection in an individual, or methods of reducing the risk of acquiring a bacterial infection in an individual, the method comprising administering to the individual a therapeutically effective amount of an antibacterial peptide described herein, and further comprising administering to the individual a second or additional therapeutic agent, thereby treating the bacterial infection, preventing the bacterial infection, or reducing the risk of acquiring the bacterial infection. In some aspects, the additional therapeutic agent is an anti-microbial agent or antibiotic. In some aspects, the additional therapeutic agent is selected from the group consisting of Penicillins (e.g., Amoxicillin, Ampicillin, Piperacillin); Beta-lactam/beta-lactamase inhibitors and combinations (e.g., Amoxicillin/clavulanate, Ampicillin/sulbactam, Ceftazidime/avibactam, Ceftolozane/tazobactam, Piperacillin/tazobactam, Meropenem/RPX-7009, Imipenem/cilastatin/relebactam); Cephalosporins (first generation Cephalosporins such as, e.g., Cefazolin; second generation Cephalosporins such as, e.g., Cefuroxime; third generation Cephalosporins such as, e.g., Ceftriaxone, Ceftazidime. and Cefotaxime; fourth generation Cephalosporins such as, e.g., Cefepime; fifth generation Cephalosporins such as, e.g., Ceftaroline and Ceftobiprole); Carbepenems (e.g., Doripenem, Imipenem/cilastatin. Meropenem, Ertapenem); monobactams (e.g., Aztreonam); Aminoglycosides (e.g., Gentamicin, Tobramycin, Amikacin, Arbekacin, Plazomicin); Tetracyclines/glycylcyclines (e.g., Tigecycline, Doxycycline, Minocycline, Eravacycline); Lincosamides (e.g., Clindamycin); Quinolones (e.g., Ciprofloxacin, Levofloxacin, Moxifloxacin, Garenoxacin, Ofloxacin, Sitafloxacin, Delafloxacin); Diaminopyrimidines (e.g., Trimethoprim/sulfamethoxazole); Phosphomycins (e.g., Fosfomycin); Polymyxins (e.g., Colistin, Polymyxin B); Dihydrofolate reductase inhibitors (e.g., Trimethoprim, Trimethoprim/sulfinethoxazole); and Nitroimidazoles (e.g., Metronidazole).

In certain embodiments, an antibacterial peptide described herein is administered in combination with an antipseudomonal beta-lactam (e.g., ticarcillin-clavulanate or piperacillin-tazobactam) to a subject in need thereof, e.g., for treating, preventing, or reducing the risk of a bacterial infection caused by Pseudomonas aeruginosa. In certain embodiments, an antibacterial peptide described herein is administered in combination with an aminoglycoside (e.g., tobramycin) to a subject in need thereof, e.g., for treating, preventing, or reducing the risk of a bacterial infection caused by Pseudomonas aeruginosa. In certain embodiments, an antibacterial peptide described herein is administered in combination with an antipseudomonal beta-lactam and an aminoglycoside to a subject in need thereof, e.g., for treating, preventing, or reducing the risk of a bacterial infection caused by Pseudomonas aeruginosa.

### Articles of Manufacture

In another aspect described herein, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a peptide described herein (e.g. a peptide that binds to a lipopolysaccharide). The label or package insert indicates that the composition is used for treating the condition of choice. Moreover, the article of manufacture may comprise (a) a first container with a composition contained therein, wherein the composition comprises a peptide described herein (e.g. a peptide that binds to a lipopolysaccharide); and (b) a second container with a composition contained therein, wherein the composition comprises a further cytotoxic or otherwise therapeutic agent. The article of manufacture in this aspect described herein may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

The following are exemplary, non-limiting embodiments of the invention.

Embodiment No. 1: A peptide of Formula I:

R¹—X¹-Pro-X⁷—X³—X⁴—X⁵-Arg-X⁶-Leu-X⁷-Lys-X⁸-Gly-Leu-Leu-Arg-R²(SEQ ID NO:66) (Formula I)

wherein,

R¹ is acetyl or is absent;

X¹, X², X³, X⁴, X⁵, X⁶, and X⁷ are each independently a natural or non-natural amino acid residue;

X is tryptophan or histidine; and

R² is amino or is absent.

Embodiment No. 2: A peptide of Formula II:

R¹—X¹-Pro-X²—X³—X⁴—X⁵-Arg-X⁶-Leu-X⁷-Lys-X⁸-Gly-Leu-R²(SEQ ID NO: 74); (Formula II)

wherein,

R¹ is acetyl or is absent;

X¹, X², X³, X⁴, X⁵, X⁶, and X⁷ are each independently a natural or non-natural amino acid residue;

X⁸ is tryptophan or histidine; and

R² is amino or is absent.

Embodiment No. 3: The peptide of any one of embodiments 1-2, wherein X¹ is tyrosine, lysine, alanine, phenylalanine, tryptophan, or arginine.

Embodiment No. 4: The peptide of any one of embodiments 1-3, wherein X¹ is tyrosine.

Embodiment No. 5: The peptide of any one of embodiments 1-3, wherein X¹ is lysine.

Embodiment No. 6: The peptide of any one of embodiments 1-5, wherein X² is methionine, N-methylmethionine, norleucine, alanine, leucine, phenylalanine. N-methylphenylalanine, homophenylalanine, (S)-2,3-diaminopropionic acid, or tryptophan.

Embodiment No. 7: The peptide of any one of embodiments 1-6, wherein X² is methionine.

Embodiment No. 8: The peptide of any one of embodiments 1-6, wherein X² is N-methylmethionine.

Embodiment No. 9: The peptide of any one of embodiments 1-6, wherein X² is norleucine.

Embodiment No. 10: The peptide of any one of embodiments 1-9, wherein X³ is threonine, allo-threonine, serine, asparagine, (S)-2,3-diaminopropionic acid, (S)-2,3-diaminobutyric acid, homoserine, or alanine.

Embodiment No. 11: The peptide of any one of embodiments 1-10, wherein X³ is threonine.

Embodiment No. 12: The peptide of any one of embodiments 1-10, wherein X³ is allo-threonine.

Embodiment No. 13: The peptide of any one of embodiments 1-10, wherein X³ is serine.

Embodiment No. 14: The peptide of any one of embodiments 1-10, wherein X³ is asparagine.

Embodiment No. 15: The peptide of any one of embodiments 1-10, wherein X³ is 2,3-diaminopropionic acid.

Embodiment No. 16: The peptide of any one of embodiments 1-10, wherein X³ is homoserine.

Embodiment No. 17: The peptide of any one of embodiments 1-16, wherein X⁴ is alanine, 2-aminobutyric acid, methionine, N-methylmethionine, phenylalanine, N-methylphenylalanine. N-methylalanine, tyrosine, (S)-2-aminoheptanoic acid, 2-amino-2-methylpropanoic acid, (S)-2,3-diaminopropionic acid, tryptophan, or arginine.

Embodiment No. 18: The peptide of any one of embodiments 1-17, wherein X⁴ is alanine.

Embodiment No. 19: The peptide of any one of embodiments 1-17, wherein X⁴ is 2-aminobutyric acid.

Embodiment No. 20: The peptide of any one of embodiments 1-17, wherein X⁴ is methionine.

Embodiment No. 21: The peptide of any one of embodiments 1-17, wherein X⁴ is phenylalanine.

Embodiment No. 22: The peptide of any one of embodiments 1-17, wherein X⁴ is N-methylalanine.

Embodiment No. 23: The peptide of any one of embodiments 1-22, wherein X⁵ is arginine, omithine, glutamine, or lysine.

Embodiment No. 24: The peptide of any one of embodiments 1-23, wherein X⁵ is arginine.

Embodiment No. 25: The peptide of any one of embodiments 1-23, wherein X⁵ is ornithine.

Embodiment No. 26: The peptide of any one of embodiments 1-23, wherein X⁵ is glutamine.

Embodiment No. 27: The peptide of any one of embodiments 1-26, wherein X⁶ is phenylalanine, homophenylalanine, (S)-3-([1,1′-biphenyl]-4-yl)-2-aminopropanoic acid, (S)-2-amino-2-(naphthalen-1-yl)acetic acid, tyrosine, or tryptophan.

Embodiment No. 28: The peptide of any one of embodiments 1-27, wherein X⁶ is phenylalanine.

Embodiment No. 29: The peptide of any one of embodiments 1-27, wherein X⁶ is (S)-3-([1,1′-biphenyl]-4-yl)-2-aminopropanoic acid.

Embodiment No. 30: The peptide of any one of embodiments 1-27, wherein X⁶ is (S)-2-amino-2-(naphthalen-1-yl)acetic acid.

Embodiment No. 31: The peptide of any one of embodiments 1-27, wherein X⁶ is tyrosine.

Embodiment No. 32: The peptide of any one of embodiments 1-31, wherein X⁷ is glutamic acid, glutamine, alanine, serine, homoserine, or arginine.

Embodiment No. 33: The peptide of any one of embodiments 1-32, wherein X⁷ is glutamic acid.

Embodiment No. 34: The peptide of any one of embodiments 1-32, wherein X⁷ is alanine.

Embodiment No. 35: The peptide of any one of embodiments 1-34, wherein X⁸ is tryptophan.

Embodiment No. 36: The peptide of any one of embodiments 1-35, wherein X⁸ is histidine.

Embodiment No. 37: The peptide of any one of embodiments 1-36, wherein:
R¹ is acetyl or is absent;
X¹ is tyrosine, lysine, alanine, phenylalanine, tryptophan, or arginine;
X² is methionine, N-methylmethionine, norleucine, alanine, leucine, phenylalanine. N-methylphenylalanine, homophenylalanine, (S)-2,3-diaminopropionic acid, or tryptophan;
X³ is threonine, allo-threonine, serine, asparagine, (S)-2,3-diaminopropionic acid, (S)-2,3-diaminobutyric acid, homoserine, lysine, arginine, or alanine;
X⁴ is alanine, 2-aminobutyric acid, methionine. N-methylmethionine, phenylalanine, N-methylphenylalanine, N-methylalanine, tyrosine, (S)-2-aminoheptanoic acid, 2-amino-2-methylpropanoic acid, (S)-2,3-diaminopropionic acid, tryptophan, or arginine;
X⁵ is arginine, ornithine, glutamine, 2-amino-2-methylpropanoic acid, (S)-2,3-diaminopropionic acid, or lysine;
X^{b} is phenylalanine, homophenylalanine, (S)-3-([1,1′-biphenyl]-4-yl)-2-aminopropanoic acid, (S)-2-amino-2-(naphthalen-1-yl)acetic acid, tyrosine, or tryptophan;
X⁷ is glutamic acid, glutamine, alanine, serine, homoserine, or arginine;
X⁸ is tryptophan or histidine: and
R² is amino or is absent.

Embodiment No. 38: A peptide of Formula III:

R¹-Arg-X^{a}—X^{b}—X^{c}—X^{d}-Arg-Arg-X^{e}-Leu-X^{f}—X^{g}—X^{h}-Gly-Leu-R²(SEQ ID NO: 228) (Formula III)
wherein,
R¹ is acetyl or is absent;
X^{a}, X^{b}, X^{c}, X^{d}, X^{e}, X^{f}, X^{g}, and X^{h} are each independently a natural or non-natural amino acid residue; and
R² is amino or is absent.

Embodiment No. 39: The peptide of embodiment 38, wherein X^{a} is proline or (S)-piperidine-2-carboxylic acid.

Embodiment No. 40: The peptide of embodiment 38 or 39, wherein X^{b} is methionine. N-methylmethionine, homophenylalanine or (S)-2-amino-5-phenylpentanoic acid.

Embodiment No. 41: The peptide of any one of embodiments 38 to 40, wherein X^{c} is threonine or (S)-2,3-diaminopropionic acid.

Embodiment No. 42: The peptide of any one of embodiments 38 to 41, wherein X^{d} is tryptophan, alanine, serine, methionine, (S)-2-aminoheptanoic acid, (S)-3-([1,1′-biphenyl]-4-yl)-2-aminopropanoic acid, O-methyl-L-serine, N-methylalanine, or 2-amino-2-methylpropanoic acid.

Embodiment No. 43: The peptide of any one of embodiments 38 to 42, wherein X^{d} is tryptophan.

Embodiment No. 44: The peptide of any one of embodiments 38 to 43, wherein X^{e} is phenylalanine, tryptophan, (S)-3-([1,1′-biphenyl]-4-yl)-2-aminopropanoic acid, or (S)-2-amino-2-(naphthalen-1-yl)acetic acid.

Embodiment No. 45: The peptide of any one of embodiments 38 to 44, wherein X^{f} is alanine, glutamic acid, or homoserine.

Embodiment No. 46: The peptide of any one of embodiments 38 to 45, wherein X^{g} is lysine or (S)-2,3-diaminobutyric acid.

Embodiment No. 47: The peptide of any one of embodiments 38 to 46, wherein X^{h} is arginine, tyrosine, histidine, tryptophan, (S)-2,3-diaminobutyric acid, or (S)-2-aminoheptanoic acid.

Embodiment No. 48: The peptide of embodiment 38, wherein:
R¹ is acetyl or is absent;
X^{a} is proline or (S)-piperidine-2-carboxylic acid;
X^{b} is methionine, N-methylmethionine, homophenylalanine or (S)-2-amino-5-phenylpentanoic acid;
X^{c} is threonine or (S)-2,3-diaminopropionic acid;
X^{d} is tryptophan, alanine, serine, methionine, (S)-2-aminoheptanoic acid, (S)-3-([1,1′-biphenyl]-4-yl)-2-aminopropanoic acid, O-methyl-L-serine, N-methylalanine, or 2-amino-2-methylpropanoic acid;
X^{e} is phenylalanine, tryptophan, (S)-3-([1,1′-biphenyl]-4-yl)-2-aminopropanoic acid, or (S)-2-amino-2-(naphthalen-1-yl)acetic acid;
X^{f} is alanine, glutamic acid, or homoserine;
X^{g} is lysine or (S)-2,3-diaminobutyric acid;
X^{b} is Arg, tyrosine, histidine, tryptophan, (S)-2,3-diaminobutyric acid, or (S)-2-aminoheptanoic acid; and
R² is amino or is absent

Embodiment No. 49: The peptide of any one of embodiments 1 to 48, wherein R¹ is acetyl.

Embodiment No. 50: The peptide of any one of embodiments 1 to 48, wherein R¹ is absent.

Embodiment No. 51: The peptide of any one of embodiments 1-50, wherein R² is amino.

Embodiment No. 52: The peptide of any one of embodiments 1-50, wherein R² is absent.

Embodiment No. 53: A peptide comprising an amino acid corresponding to one of SEQ ID NOs: 5-65, 68-73, or 78-108.

Embodiment No. 54: A peptide comprising an amino acid corresponding to SEQ ID NOs: 14, 48, 49, 50, 51, 52, 53, 55, 57, 58, 59, 60, 62, 63, 64, and 65 or 69-73 or to SEQ ID NOs: 11, 12, 14, 48, 49, 50, 51, 52, 53, 55, 57, 58, 59, 60, 62, 63, 64, 65, 68, 69, 70, 71, 72, or 73.

Embodiment No. 55: A peptide comprising an amino acid corresponding to one of SEQ ID NOs: 200-227.

Embodiment No. 56: The peptide of any one of embodiments 1-55, wherein the peptide binds to a lipopolysaccharide.

Embodiment No. 57: The peptide of embodiment 56, wherein the peptide binds to the lipid A portion of a lipopolysaccharide.

Embodiment No. 58: The peptide of embodiment 55 or 56, wherein the peptide has a lipopolysaccharide-binding affinity in terms of Kd of ≤100 μM as measured by biolayer interferometry.

Embodiment No. 59: The peptide of embodiment 55 or 56, wherein the peptide has a lipopolysaccharide-binding affinity in terms of Kd of ≤10 μM as measured by biolayer interferometry.

Embodiment No. 60: The peptide of embodiment 55 or 56, wherein the peptide has a lipopolysaccharide-binding affinity in terms of Kd of ≤1 μM as measured by biolayer interferometry.

Embodiment No. 61: A pharmaceutical composition comprising a peptide of any one of embodiments 1-55, and a pharmaceutically acceptable excipient.

Embodiment No. 62: The pharmaceutical composition of embodiment 61, further comprising an additional therapeutic agent.

Embodiment No. 63: The pharmaceutical composition of embodiment 62, wherein the additional therapeutic agent comprises antibiotics or antiseptics.

Embodiment No. 64: A peptide of any one of embodiments 1-55, for use as therapeutically active substance.

Embodiment No. 65: A use of a peptide of any one of embodiments 1-55, for the therapeutic treatment of a bacterial infection.

Embodiment No. 66: A use of a peptide of any one of embodiments 1-55, for the preparation of a medicament for the therapeutic treatment of a bacterial infection.

Embodiment No. 67: A peptide of any one of embodiments 1-55, for the therapeutic treatment of a bacterial infection.

Embodiment No. 68: A method for the therapeutic treatment of a bacterial infection, which method comprises administering a therapeutically effective amount of a peptide of any one of embodiments 1-55.

Embodiment No. 69: The method of embodiment 68, further comprising administering an additional therapeutic agent.

Embodiment No. 70: The method of embodiment 69, wherein the additional therapeutic agent comprises antibiotics or antiseptics.

Embodiment No. 71: The use of embodiment 65 or 66, or the peptide of embodiment 67, or the method of any one of embodiments 68-71, wherein the bacterial infection is caused by a Gram-negative bacterium.

Embodiment No. 72: The use of 65 or 66, or the peptide of embodiment 67, or the method of any one of embodiments 68-71, wherein the bacterial infection is caused by a Gram-negative bacterium selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp.

Embodiment No. 73: The use of embodiment 65 or 66, or the peptide of embodiment 67, or the method of any one of embodiments 68-71, wherein the bacterial infection is caused by a Gram-negative bacterium selected from the group consisting of Escherichia coli, Klebsiella pneumoniae, Pseudomonas aeruginosa, Acinetobacter baumannii, Enterobacter cloacae.

Embodiment No. 74: The use of embodiment 65 or 66, or the peptide of embodiment 67, or the method of any one of embodiments 68-71, wherein the bacterial infection is caused by a Gram-negative bacterium selected from the group consisting of Escherichia coli, Klebsiella pneumoniae, Pseudomonas aeruginosa, and Acinetobacter baumannii.

Embodiment No. 75: The use of embodiment 65 or 66, or the peptide of embodiment 67, or the method of any one of embodiments 68-71, wherein the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

Embodiment No. 76: The peptide of any one of embodiments 1-55, conjugated to a therapeutic agent.

Embodiment No. 77: The peptide of any one of embodiments 1-55, conjugated to a label.

Embodiment No. 78: The peptide of embodiment 77, wherein the label is a radioisotope, a fluorescent dye, or an enzyme.

Embodiment No. 79: A method of producing the peptide of any one of embodiments 1-55, comprising chemically synthesizing the peptide.

Embodiment No. 80: An isolated nucleic acid encoding the peptide of any one of embodiments 1-55.

Embodiment No. 81: An expression vector encoding the nucleic acid molecule of embodiment 80.

Embodiment No. 82: A cell comprising the expression vector of embodiment 81.

Embodiment No. 83: A method of producing the peptide of any one of embodiments 1-55, comprising culturing the cell of embodiment 82 and recovering the peptide from the cell culture.

Embodiment No. 84: A method of treating an individual having a bacterial infection comprising administering to the individual an effective amount of a peptide that binds to a lipopolysaccharide comprising an amino acid sequence having a homology of ≥50% with SEQ ID NO: 1.

Embodiment No. 85: The method of embodiment 84, wherein the peptide binds to a lipopolysaccharide of a Gram-negative bacterium.

Embodiment No. 86: The method of embodiment 85, wherein the Gram-negative bacterium is selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp.

Embodiment No. 87: The method of embodiment 85, wherein the Gram-negative bacterium is selected from the group consisting of Escherichia coli, Klebsiella pneumoniae, Pseudomonas aeruginosa, Acinetobacter baumannii, and Enterobacter cloacae.

Embodiment No. 88: The method of embodiment 85, wherein the Gram-negative bacterium is selected from the group consisting of Escherichia coli, Klebsiella pneumoniae, Pseudomonas aeruginosa, and Acinetobacter baumannii.

Embodiment No. 89: The method of any one of embodiments 84-88, wherein the peptide binds to the lipid A portion of a lipopolysaccharide.

Embodiment No. 90: The method of any one of embodiments 84-88, wherein the peptide has a lipopolysaccharide-binding affinity in terms of Kd of ≤≤100 μM as measured by biolayer interferometry.

Embodiment No. 91: The method of any one of embodiments 84-88, wherein the peptide has a lipopolysaccharide-binding affinity in terms of Kd of ≤≤10 μM as measured by biolayer interferometry.

Embodiment No. 92: The method of any one of embodiments 84-88, wherein the peptide has a lipopolysaccharide-binding affinity in terms of Kd of ≤1 μM as measured by biolayer interferometry.

Embodiment No. 93: The method of any one of embodiments 84-92, wherein the peptide binds to a lipopolysaccharide selectively over a bacterial membrane phospholipid.

Embodiment No. 94: The method of embodiment 93, wherein the bacterial membrane phospholipid is phosphatidylethanolamine, phosphatidylglycerol, or cardiolipin.

Embodiment No. 95: The method of any one of embodiments 84-88, wherein the peptide has an IC₅₀ of ≤10 μM against a Gram-negative bacterium, as measured by an in vitro bacterial growth assay in LB or Mueller Hinton II cation-adjusted broth at 37° C.

Embodiment No. 96: The method of any one of embodiments 84-88, wherein the peptide has an IC₅₀ of ≤1 μM against a Gram-negative bacterium, as measured by an in vitro bacterial growth assay in LB or Mueller Hinton II cation-adjusted broth at 37° C.

Embodiment No. 97: The method of any one of embodiments 84-88, wherein the peptide has an IC₅₀ of ≤100 nM against a Gram-negative bacterium, as measured by an in vitro bacterial growth assay in LB or Mueller Hinton II cation-adjusted broth at 37° C.

Embodiment No. 98: The method of any one of embodiments 84-88, wherein the peptide has an MIC of ≤500 μM against a Gram-negative bacterium, as measured by an in vitro bacterial growth assay in LB or Mueller Hinton II cation-adjusted broth at 37° C.

Embodiment No. 99: The method of any one of embodiments 84-88, wherein the peptide has an MIC of ≤50 μM against a Gram-negative bacterium, as measured by an in vitro bacterial growth assay in LB or Mueller Hinton II cation-adjusted broth at 37° C.

Embodiment No. 100: The method of any one of embodiments 84-88, wherein the peptide has an MIC of ≤5 μM against a Gram-negative bacterium, as measured by an in vitro bacterial growth assay in LB or Mueller Hinton II cation-adjusted broth at 37° C.

Embodiment No. 101: The method of any one of embodiments 95-100, wherein the Gram-negative bacterium is selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp.

Embodiment No. 102: The method of any one of embodiments 95-100, wherein the Gram-negative bacterium is selected from the group consisting of Escherichia coli, Klebsiella pneumoniae, Pseudomonas aeruginosa, Acinetobacter baumannii, and Enterobacter cloacae.

Embodiment No. 103: The method of any one of embodiments 95-100, wherein the Gram-negative bacterium is selected from the group consisting of Escherichia coli, Klebsiella pneumoniae, Pseudomonas aeruginosa, and Acinetobacter baumannii.

Embodiment No. 104: The method of any one of embodiments 84-103, wherein the peptide has a length of 10-20 amino acid residues.

Embodiment No. 105: The method of any one of embodiments 84-103, wherein the peptide has a length of 12-18 amino acid residues.

Embodiment No. 106: The method of any one of embodiments 84-103, wherein the peptide has a length of 14-16 amino acid residues.

Embodiment No. 107: The method of any one of embodiments 84-106, wherein the peptide comprises an amino acid sequence having a homology of ≥60% with SEQ ID NO: 1.

Embodiment No. 108: The method of any one of embodiments 84-106, wherein the peptide comprises an amino acid sequence having a homology of ≥70% with SEQ ID NO: 1.

Embodiment No. 109: The method of any one of embodiments 84-106, wherein the peptide comprises an amino acid sequence having a homology of ≥80% with SEQ ID NO: 1.

Embodiment No. 110: The method of any one of embodiments 84-106, wherein the peptide comprises an amino acid sequence having a homology of ≥90% with SEQ ID NO: 1.

Embodiment No. 111: The method of any one of embodiments 84-106, wherein the peptide comprises an amino acid sequence having a homology of ≥95% with SEQ ID NO: 1.

Embodiment No. 112: The method of embodiment 84, wherein the peptide comprises an amino acid sequence selected from the group consisting of SEQ ID NOs: 14, 48, 49, 50, 51, 52, 53, 55, 57, 58, 59, 60, 62, 63, 64, and 65 or 69-73.

Embodiment No. 113: The method of embodiment 84, wherein the bacterial infection is caused by a Gram-negative bacterium.

Embodiment No. 114: The method of embodiment 113, wherein the Gram-negative bacterium is selected from the group consisting of Escherichia coli, Klebsiella spp., Pseudomonas spp., Enterobacter spp., Bordatella spp., Burkholderia sp., Stenotrophomonas maltophilia, Bacteroides spp., Campylobacter spp., Francisella tularensis, Helicobacter pylori, Legionella spp., and Vibrio spp.

Embodiment No. 115: The method of embodiment 113, wherein the Gram-negative bacterium is selected from the group consisting of Escherichia coli, Klebsiella pneumoniae, Pseudomonas aeruginosa, Acinetobacter baumannii, and Enterobacter cloacae.

Embodiment No. 116: The method of embodiment 99, wherein the Gram-negative bacterium is selected from the group consisting of Escherichia coli, Klebsiella pneumoniae, Pseudomonas aeruginosa, and Acinetobacter baumannii.

Embodiment No. 117: The method of any one of embodiments 84-116, wherein the individual is human.

Embodiment No. 118: The method of embodiment 113, wherein the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

### EXAMPLES

The following are examples of methods and compositions described herein. It is understood that various other embodiments may be practiced, given the general description provided above.

Bacterial strains and plasmids. To generate pBAD-pbgA, pbgA was amplified from uropathogenic E. coli (UPEC CFT073) and cloned into pBAD vector using Gibson assembly according to manufacturer's instructions (New England Biolabs). Mutations in pbgA were created using QuikChange II XL site-directed mutagenesis kit (Agilent Technologies) and confirmed by PCR and DNA sequencing.

Mutant strains were created using λ Red recombination (Datsenko, et al. PNASci USA 97, 6640-45, (2000)). Briefly, the kanamycin or gentamicin cassette from pKD4 was amplified with primers containing ˜50 bp nucleotide homology extensions to the gene of interest. The linear product was transformed into the appropriate background strain containing pSIM18 (Chan, et al. Nucleic Acids Res 35, e64, (2007)), recovered for 4 hr at 37° C. and selected on media containing 50 μg/mL kanamycin or 12.5 μg/mL chloramphenicol or 10 μg/mL gentamicin, as appropriate. Mutations were confirmed by PCR and sequencing. Construction of the UPEC-ΔpbgA and K-12-ΔpbgA strains resulted in single clones and the pbgA deletions were confirmed by PCR. Genomic DNA was isolated using the Blood and Cell Culture DNA Maxi kit (Qiagen) and sequenced using the Ilumina HiSeq 2000 platform to identify the suppressor. Paired-end 75 bp reads were aligned to the E. coli CFT073 genome using GSNAP version 2013-10-10 with the following parameters: −M 2-n 10-B 2-i 1—pairmax-dna=1000—terminal-threshold=1000—gmap-mode=none—clip-overlap. Variant calling was performed using an in-house bioinformatics pipeline utilizing R and Bioconductor packages, GenomicRanges, GenomicAlignments, VariantTools, and gmapR, with a required base quality score for variant tallying of 30. (See Lawrence, et al. PLoS Comput Biol 9, e1003118, (2013).) No single-nucleotide variants or indels were found, but mapping confirmed this strain lacked the pbgA gene and identified a large (>560 kb base pair) genomic duplication that straddles the origin (nucleotide positions 1-255,360 and 4,933,251-5,242,300). The mechanism of pbgA suppression in this strain has not yet been determined, but αcpT, a reported multi-copy suppressor of ΔpbgA⁴⁰, is not duplicated in UPEC-ΔpbgA.

The conditional pbgA strain, ΔpbgA::pBADpbgA, was created by inserting pBADpbgA at the attB site in BW25113 followed by deletion of the native copy of pbgA (Datsenko et al.; Diederich, et al., Plasmid 28, 14-24 (1992).) Briefly, pbgA was cloned into pBAD28 using standard methods. pBADpbgA was amplified from pBAD28-pbgA and sub-cloned into pLDR9. pLDR9-pBADpbgA was digested with NotI, ligated, and transformed into BW25113 expressing pLDR8. PCR and DNA sequencing confirmed insertion of pBADpbgA at the attB site. After integration of pBADpbgA, the native copy of pbgA was deleted using λ Red recombination as described above.

The triple ΔclsABC mutant was constructed by sequentially introducing each individual cls deletion from the Keio collection (Baba, et al. Mol Syst Biol 2, 2006 0008, (2006)) into E. coli BW25113 by P1vir transduction using standard procedures (Miller, J. H. Experiments in molecular genetics. (Cold Spring Harbor Laboratory, 1972). Deletions were confirmed by PCR.

pFhuAΔC/Δ4L was constructed by synthesizing the fhuA coding sequence lacking the N-terminal cork domain, Δ1-160, and extracellular loops L3, L4, L5, and L11 (Mohammad, et al., J Biol Chem 286, 8000-8013, (2011)).fhuAΔcΔ4L was amplified with primers N3P-105 (encoding the bla constitutive promoter, ribosome binding site, and AUG start codon from pUC19 (New England BioLabs) and N3P-107, and cloning into pACYC184 with BamHI and HindIII (New England BioLabs).

Bacterial growth conditions. LB or Mueller Hinton II cation-adjusted broth (MHB II, BBL 212322) was prepared according to manufacturer's instructions and supplemented with arabinose at 0.02% for overnight growth or at indicated concentrations in the description of the figures. Bacterial cultures were grown at 37° C. When appropriate, media was supplemented with kanamycin (50 μg/mL), carbenicillin (50 μg/mL), chloramphenicol (12.5 or 25 μg/mL), hygromycin (200 μg/mL), and/or gentamicin (10 μg/mL).

Peptide sensitivity and MIC assays. Lyophilized peptides (Smartox Biotechnology, CPC Scientific. ABclonal) were solubilized to 10 mM in Tris buffer, pH 8, prior to making a 2-fold dilution series in MHBII cation adjusted broth with highest final assay concentration of 400-800 μM. Bacteria were grown to log phase and added to final OD₆₀₀˜0.001. The final EDTA concentration was 0.5 mM for E. coli strains and 0.125 mM for USA300. The final volume of 10 μL/well in a 384 well black, clear bottom plate (Corning) was spun <1 min at <250×g and then incubated at 37° C. After 4 h, an equal volume (10 μL) of BacTiter-Glo™ (Promega) was added to each well and luminescence was read after 5 min on an EnVision plate reader (PerkinElmer). To measure the minimal inhibitory concentrations (MICs), bacteria from fresh overnight plates or log phase cultures were diluted with or without EDTA and peptides as described above to final OD₆₀₀˜0.0002. Plates were incubated at 37° C. for 20 h before measuring OD₆₀₀ with an EnVision plate reader.

Recombinant protein expression and purification. Full-length (residues 1-586) of E. coli PbgA followed by a TEV cleavage site, 2xFLAG-tag and a hexahistidine tag (SEQ ID NO: 75) at the C-terminus were cloned into a modified pET52b vector. Proteins were expressed in E. coli BL21-Gold(DE3) for 48 h in TB autoinduction media at 17° C. Fifty grams of cell pellet was resuspended in 250 mL of 50 mM Tris pH 8, 300 mM NaCl, 1 μg/mL benzonase, 1 mM PMSF and Roche protease inhibitor tablets. Cells were lysed by sonication and PbgA were subsequently solubilized by addition of 1% (wt/v) lauryl maltose neopentyl glycol (LMNG) for 2 h at 4° C. under gentle agitation. Insoluble debris was pelleted by centrifugation at 18,000 rpm for 1 h, and the supernatant containing the solubilized protein was collected for affinity purification by batch-binding to 20 mL of M2-agarose FLAG resin (Sigma) for 2 h at 4° C. Unbound proteins were washed with 10 column volumes of purification buffer (50 mM Tris pH 8, 300 mM NaCl, 0.025% (wt/v) LMNG) and eluted with 5 column volumes of purification buffer supplemented with 150 μg/mL FLAG peptide (Sigma). The eluate was collected and concentrated with 100 kDa MWCO concentrators to 1 mg/mL prior to tag removal by TEV cleavage overnight at 4° C. PbgA was then concentrated to 4 mg/mL, supplemented with 1 mM NiCl₂, and injected onto a Superdex™ S200 Increase 10/300 column attached to an ÄKTA system (GE Healthcare) for size-exclusion chromatography into crystallization or SEC-MALS buffer (20 mM sodium citrate pH 5, 200 mM NaCl, 0.025% LMNG). Elution fractions corresponding to monomeric PbgA in LMNG were pooled and concentrated to 40 mg/mL for crystallization.

Crystallization, data collection and structure determination. Crystal screens in LCP were set up using 40 mg/mL PbgA and a monoolein (Sigma): phosphatidylethanolamine (E. coli PE, Avanti Polar Lipids) 99.5:0.5% m/m mixture at 40% hydration. Protein-lipid mixes were prepared at room temperature as previously described (M. Caffrey, V. Cherezov, Nat Protoc 4, 706-731 (2009)) and crystals grew in 50 nL drops surrounded by 800 nL reservoir solution. Rounds of optimization in MemMeso™ HT screens (Molecular dimensions) yielded the best-diffracting PbgA crystals that were obtained in a buffer containing 0.1 M Tris pH 8.0, 0.2 M ammonium sulfate, 40% PEG200 at 4° C., and grew to their maximum size in ˜20 days. Crystals were flashed-frozen without further cryoprotection for screening. 180° of X-ray diffraction data was collected from a single crystal at the Stanford Synchrotron Radiation Lightsource beamline SSRL12-2 at 100 K, and integrated and scaled using HKL2000 (Z. Otwinowski, W. Minor, Methods Enzymol 276, 307-326 (1997)). PbgA crystallized in the C2 space group with one monomer in the asymmetric unit. The PbgA structure was determined by molecular replacement using PHASER (A. J. McCoy et al., J Appl Crystallogr 40, 658-674 (2007)) with the PbgA periplasmic domain search model (PDB: 5I5H). Following rigid-body refinement of the periplasmic domain template, clear electron density was visible for the transmembrane domain. The model was completed manually and rebuilt through iterative refinement and omit maps using COOT (P. Emsley et al., Acta Crystallogr D Biol Crystallogr 66, 486-501 (2010)) and PHENIX (P. D. Adams et al., Acta Crystallogr D Biol Crystallogr 66, 213-221 (2010)). Secondary structure restraints were initially applied during refinement but relaxed, and TLS parameters were also employed at late stages in refinement (M. D. Winn et al., Acta Crystallogr D Biol Crystallogr 57, 122-133 (2001)). LPS was modeled only at very late stages of refinement after all protein, other lipids, and most solvent molecules were accounted for. Because reasonable completeness and data quality were available to 1.85 Å, the structure with ligands were refined against all available data until the last round of refinement, where the resolution was cut back to 2.0 Å. All structural figures were generated using PyMOL (V. S. The PyMOL Molecular Graphics System, LLC) and all density maps were calculated to 2.0 Å, where Fₒ-F_{c} maps were calculated prior to the inclusion of LPS into the refined model in order to avoid introducing model bias from this ligand.

Biolayer interferometry. Phospholipid (Avanti polar lipids) and KDO₂-lipid A (US Biological Life Sciences) stock solutions were prepared by resuspension into 25 mM Tris pH 8, 100 mM NaCl, 0.05% LMNG buffer and solubilized overnight at 4° C. Lipid stocks were diluted prior to experiments into 25 mM Tris pH 8, 100 mM NaCl, 0.5 mg/mL BSA, 0.05% LMNG. All assays were performed at 25° C. in 25 mM Tris pH 8, 100 mM NaCl, 0.5 mg/mL BSA, 0.05% LMNG. Biotinylated-LAB peptides were loaded onto SA biosensors to a response of approximately 0.5 nm. Binding to phospholipids and KDO₂-lipid A was measured at concentrations of 150, 100, 50, 25, and 10 μM with 300s association and dissociation steps. Assays were performed in triplicate on an Octet Red384 (ForteBio) and buffer and lipid signals were subtracted by using a biotin-blocked reference streptavidin (SA) biosensor. Dissociation constants for LAB_{WT} and LAB_{WT+}, interactions with KDO₂-lipidA were estimated by plotting response values at equilibrium as a function of concentration and fit to a global specific binding with Hill slope model in Prism (Graphpad Software).

### Example 1: Purification and Lipid-Dependent Crystallization of PbgA

The structure of full-length PbgA was determined to gain insight into its essential function. Full-length PbgA from E. coli and Salmonella typhimurium were monomeric when purified in mild detergent as determined by size-exclusion chromatography with multi-angle light scattering (SEC-MALS). PbgA crystals were obtained upon reconstitution into monoolein (MO)-based lipidic cubic phases (LCP), and addition of the zwitterionic lipid phosphatidylethanolamine (PE) into the LCP matrix was essential for obtaining diffraction data that extended beyond 2.0 Å resolution. The high-resolution crystal structure of full-length E. coli PbgA was determined and found to display a membrane bilayer-like arrangement characteristic of proteins reconstituted into LCP.

### Example 2: Structure of PbgA in a Membrane-Like Environment

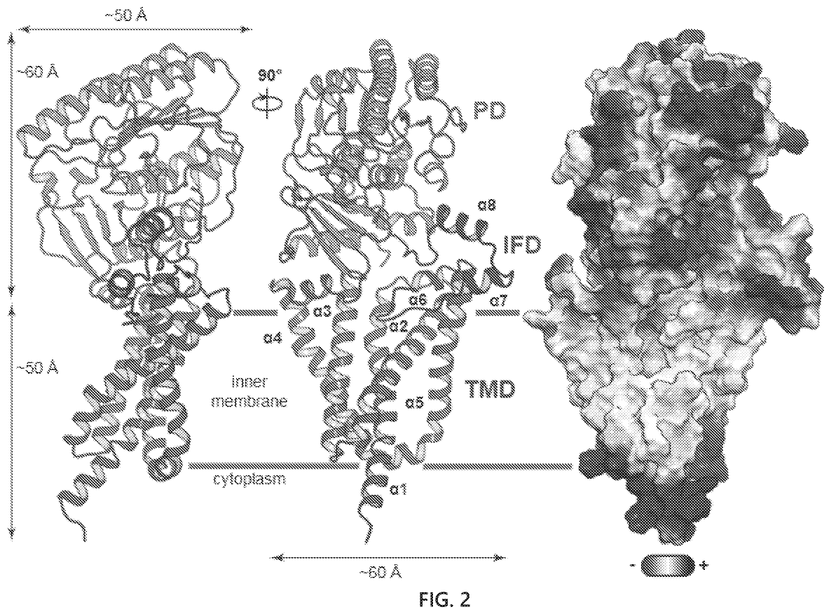

The overall structure of PbgA is reminiscent of a baseball glove (FIG. 2). Five N-terminal TM-helices form a convex palm upon which the C-terminal periplasmic domain (PD) sits with extended beta-sheets and loops forming the fingers and inner surface of the glove (FIG. 2). A ˜65 residue long linker (the interfacial domain (IFD)) connects the TM domain (TMD) and PD by forming a compact helix-turn-helix-turn-helix module which fuses the membrane and soluble domains together (FIG. 2). The electrostatic surface potential of PbgA illustrates its relative positioning within the IM bilayer and provides the impression that the TMD, IFD, and PD are welded together (FIG. 2).

A number of observations emerged from structural analysis of full-length PbgA. First, the TMD and PD are tightly structurally coupled, burying ˜740 Å² of mainly hydrophobic surface contacts, which increases to ˜1760 Å² when IFD interactions are also considered. Second, the IFD forms a compact and integrated structural module that is not a simple unstructured linker as previously suggested. Third, the putative CL-binding site hypothesized to exist within the PD (H. Dong et al., Sci Rep 6, 30815 (2016)) is distant from the IM and shows no sign of extra electron density or plausible structural rearrangements that might permit PL access. Fourth, simple geometric considerations and crystal packing analysis supports the conclusion that PbgA is a monomeric protein, consistent with solution-based studies. Fifth, a remarkable extra electron density consistent with a bound LPS molecule was observed at the IFD-membrane interface in both the X-ray and XFEL PbgA crystal structures (Example 3).

### Example 3: LPS is Bound to PbgA

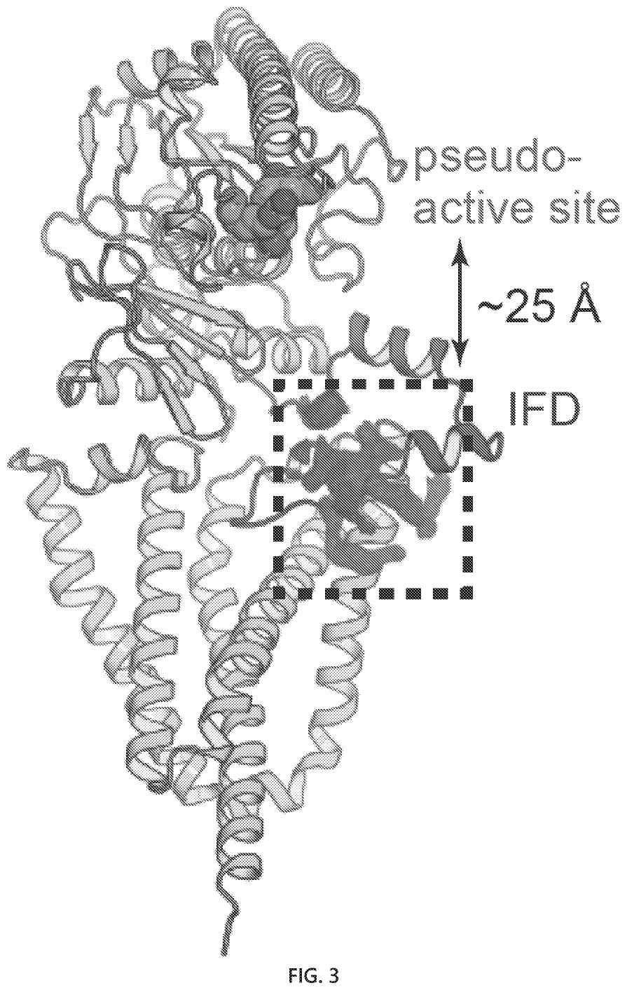

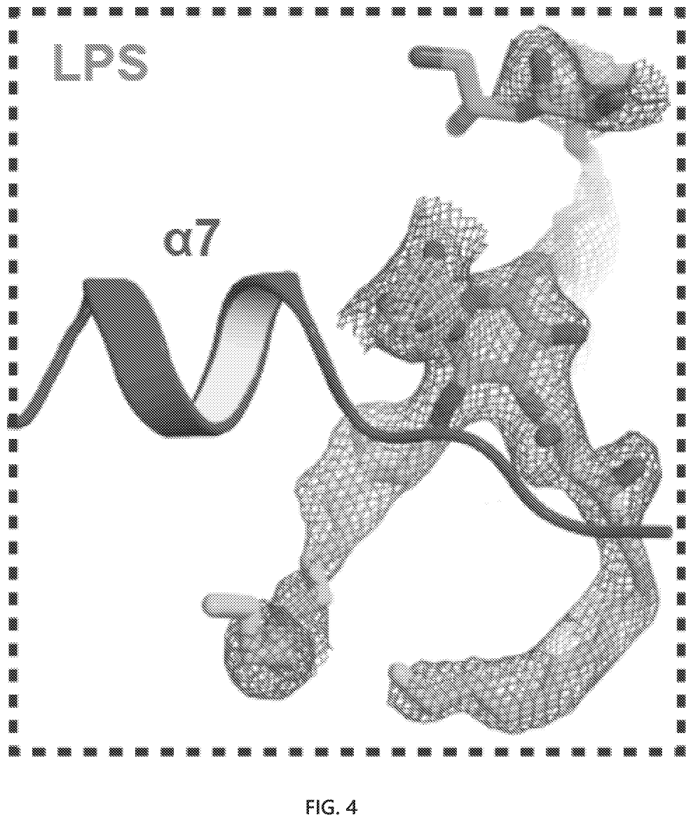

Within the PbgA crystal structure, a strong extra electron density was observed along the periplasmic membrane leaflet cradled against the IFD (FIGS. 3 and 4). This bilobal feature sits at the lipid-aqueous interface ˜25 Å away from a defunct hydrolase site (FIGS. 3 and 4).

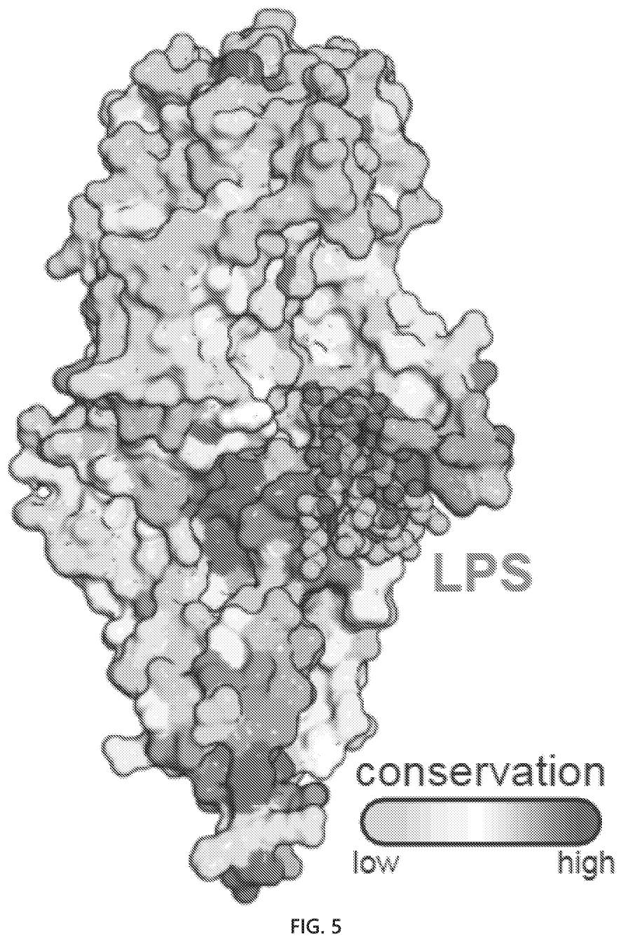

The distinctive bilobal structure of the unidentified density warranted consideration that LPS may remain bound to PbgA through purification and reconstitution into the MO-based crystallization matrix. Lipid A was subsequently found to rationalize the unassigned electron density (FIGS. 3 and 4) where the electrostatic surface potential of PbgA matches the amphipathic features expected to bind lipid A (FIG. 2). Sequence analysis (H. Ashkenazy et al., Nucleic Acids Res 44, W344-350 (2016)) further identifies high conservation of the lipid A binding region across PbgA homologs (FIG. 5).

Thus, a single co-purifying LPS molecule is bound to PbgA along the periplasmic leaflet of the membrane.

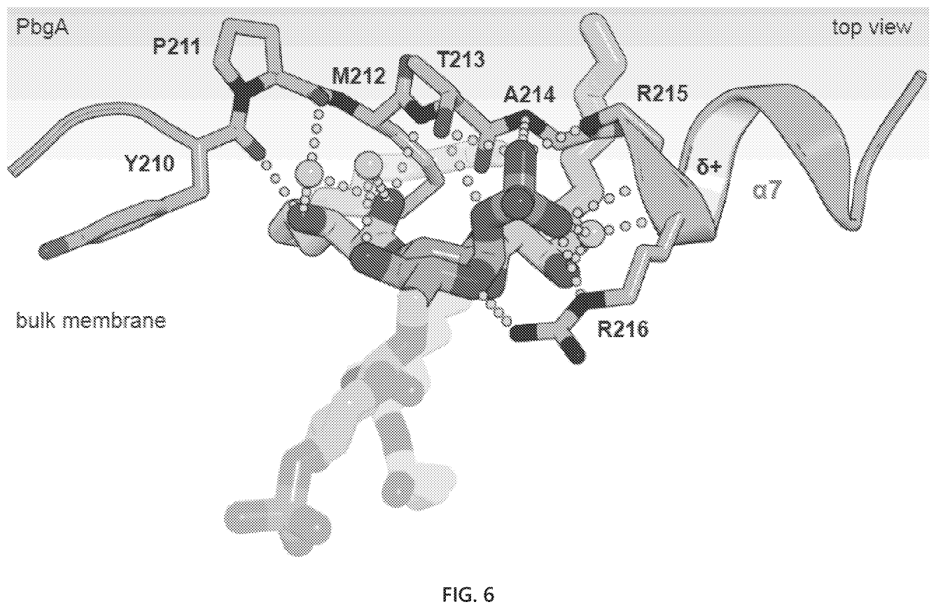

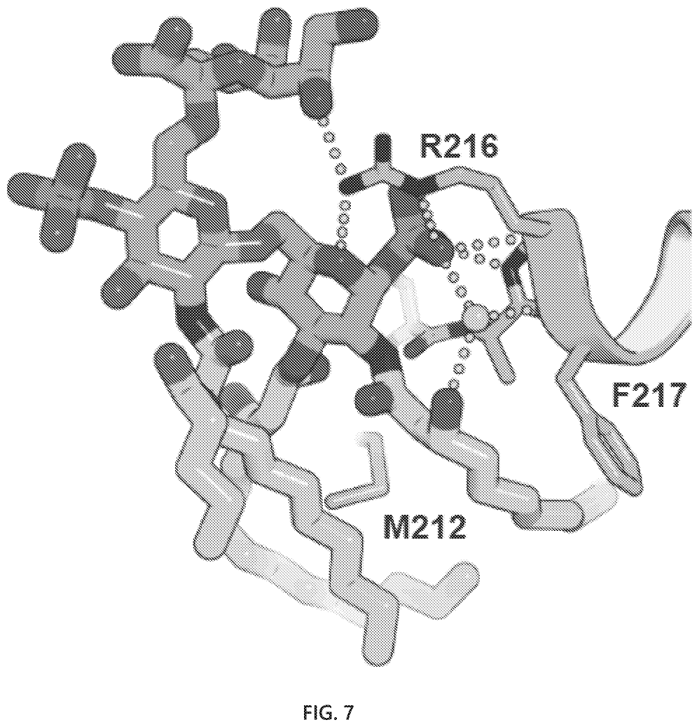

The high-resolution LPS-PbgA complex is shown in (FIGS. 6 and 7). PbgA engages LPS through eight residues that precede and form part of the IFD-helix α7, ₂₁₀YPMTARRF₂₁₇ (SEQ ID NO: 76) (FIG. 6). Using this simple linear-motif, PbgA forms an elaborate interaction network directly to a characteristic feature of LPS, a single phosphorylated D-glucosamine (GlcNac) moiety of lipid A (FIG. 6). This lipid A coordination strategy is unprecedented (H. M. Berman et al., The Protein Data Bank. Nucleic Acids Res 28, 235-242 (2000)) and sharply contrasts with the selective LPS transporter MsbA and the high-affinity toll-like receptor 4 (TLR4) which engulf LPS to exploit the bivalent nature of lipid A chemistry (W. Mi et al., Nature 549, 233-237 (2017); H. Ho et al., Nature 557, 196-201 (2018); B. S. Park et al., Nature 458, 1191-1195 (2009)).

Within the LPS-PbgA complex, Phe217 anchors the α7 helix into the membrane while its backbone hydrogen bonds through a water to the R-3-hydroxymyristoyl and 1′-phospho-GlcNac of lipid A (FIGS. 6 and 7). The backbone amides of Arg216 and Arg215 complex directly to the 1′-phospho-group of the GlcNac moiety, which is stabilized further by the α7 helical dipole (FIGS. 6 and 7). The Arg216 side-chain extends to the 5′-ether and 1′-phospho-positions of GlcNac, as well as to the 07-hydroxyl of the proximal keto-deoxyoctulosonate (KDO) sugar (FIGS. 6 and 7), although this guanidino group is not conserved. Ala214 provides a key spatial link to the₂₁₀YPMT₂₁₃ segment (SEQ ID NO: 77) which allows the backbone of Thr213 to engage the 3′-linked R-3-hydroxymyristoyl group of lipid A, and the Thr213 hydroxyl to interact with the 1′-hydroxyl and 1′-phospho-positions of the GlcNac substituent (FIG. 6). Within the membrane phase, Met212 provides hydrophobic and van der Waals contacts by wedging in-between the 2′-linked and 3′-linked R-3-hydroxymyristoyl groups (FIGS. 6 and 7). Finally, the backbone Pro211 and Tyr 210 bond to the 3′-linked R-3-hydroxymyristoyl substituent of lipid A, where Pro211 mediates this interaction through a water molecule (FIG. 6). Overall, a dense 14-point interaction network was observed that allows PbgA to bind to LPS within a bulk PL membrane. PbgA may achieve selective lipid A-coordination primarily through 10 backbone- and water-mediated interactions to a single phospho-GlcNAc unit of lipid A.

### Example 4: PbgA-Inspired Peptides Bind LPS Selectively

The LPS coordination strategy employed by PbgA sharply contrasts with other LPS-selective binding proteins and bacterial OM proteins-LPS complexes, since these proteins generally engage multiple acyl chains and the phospho-disaccharide of lipid A simultaneously (W. Mi et al., Nature 549, 233-237 (2017); H. Ho et al., Nature 557, 196-201 (2018); B. S. Park et al., Nature 458, 1191-1195 (2009); A. D. Ferguson et al., Science 282, 2215-2220 (1998); W. Arunmanee et al., Proc Natl Acad Sci USA 113, E5034-5043 (2016)). Because PbgA only contacts the minimal and stable chemistry which defines lipid A, PbgA appears competent to bind any LPS species present within the IM, including those modified by PMX-resistance enzymes.

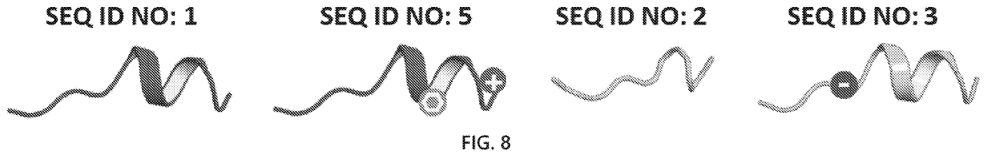

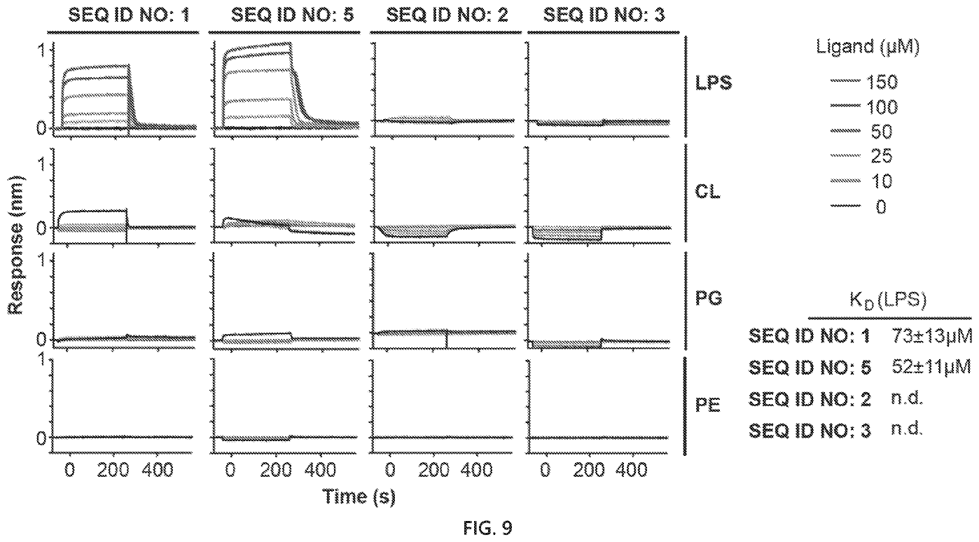

Based on the intriguing chemical characteristics of the LPS-PbgA interface, the possibility that a linear peptide derived from PbgA might selectively bind to LPS in vitro was considered. A peptide encompassing the lipid A-binding (LAB)-motif (₂₀₉SYPMTARRFLEKHGLLD₂₂₅; SEQ ID NO: 67) with an additional Gly-Ser-linker and biotin modification was generated and used in an interferometry-based assay. This synthetic lipid A-binding (LAB_{WT}) peptide (SEQ ID NO: 1) bound LPS selectively over the major PLs found in E. coli (K_{d}˜75 μM; FIGS. 8 and 9). Two peptides intended to destabilize essential lipid A binding determinants were tested as controls. Neither a peptide lacking the α7 helix (LAB_{Δα7}; SEQ ID NO: 2) nor a peptide containing the T213D mutation equivalent (LAB_{T213D}; SEQ ID NO: 3) showed detectable binding to LPS or PLs above background (FIGS. 8 and 9).

A peptide intended to promote membrane association (LAB_{WT+}; SEQ ID NO: 68) was designed because excising the LAB_{WT} sequence from its native protein and membrane context should reduce its intrinsic affinity for lipid A. This LAB_{WT+} peptide variant (SEQ ID NO: 68) introduced the H221W and D225R mutation equivalents (₂₀₉SYPMTARRFLEKWGLLR₂₂₅; SEQ ID NO: 68) and showed ˜1.5-fold improved affinity towards LPS (K_{d} ˜55 μM) while also maintaining selectivity over PL binding (FIGS. 8 and 9). It is notable that the synthetic LAB_{WT} (SEQ ID NO: 1) and LAB_{WT+} (SEQ ID NO: 68) peptides selectively bound to LPS over PLs despite containing multiple arginine residues because basic side-chains are typically employed by selective PL-binding domains (M. A. Lemmon, Nat Rev Mol Cell Biol 9, 99-111 (2008)). Thus, an interferometry-based in vitro assay established that the isolated lipid A-binding motif identified from the LPS-PbgA crystal structure represents a peptide capable of selective lipid A coordination.

### Example 5: LAB-Peptides Inhibit Growth of Escherichia coli

Because PMXs kill Gram-negative bacteria by binding to lipid A, the antimicrobial activity of the LAB_{WT+} peptide (SEQ ID NO: 68) was explored. The LAB_{WT} peptide (SEQ ID NO: 1) significantly impacted the growth of two E. coli strains (E. coli imp4213 and E. coli+FhuAΔC/Δ4L) known to promote the penetration of large molecules across the OM. However, the LAB_{WT} peptide (SEQ ID NO: 1) failed to inhibit the growth of a wild-type E. coli strain. The LAB_{WT+} peptide (SEQ ID NO: 68) also failed to inhibit the growth of a wild-type E. coli strain.

TABLE 2
Peptide SEQ ID NO. 68 inhibits growth of OM permeabilized E. coli

| Strain | Strain Description | MIC (μM) |
|---|---|---|
| E. coli BW25113 | Gram-negative bacterium, wild-type strain | >800 |
| S. aureus USA300 | Gram-positive bacterium, wild-type strain | >800 |
| E. coli BW25113 + EDTA | Permeabilized OM (chemical) | 200 |
| E. coli imp4213 | Permeabilized OM (genetic) | 25 |
| E. coli + FhuAΔC/Δ4L | Permeabilized OM (genetic) | 50 |
| E. coli ΔwaaD | Permeabilized G (genetic) | 400 |

¹ MIC, minimal inhibitory concentration, is the lowest concentration of compound that results in complete growth inhibition. LAB_{wr+} was synthesized with an N-tenninal acetyl and C-terminal amide.
² 0.5 mM EDTA included to penneabilizes E. coli OM.
³ E. coli imp4213 encodes a mutant lptD leading to OM penneabilization,
⁴ E. coli ΔwaaD possess truncated LPS leading to OM penneabilization.
⁵ E. coli FhuAΔCΔ4L produces large porins in the OM leading to permeabilization.

It was reasoned that the relatively large molecular weight (>2 kDa) and limited α-helical propensity of the linear LAB_{WT} peptide (SEQ ID NO: 1) might limit its access to LPS in the bacterial OM. A cell growth inhibition assay was performed in the presence of 0.5 mM Methylenediaminetetraacetic acid (EDTA), a chemical that destabilizes the OM by disrupting divalent cation-LPS interactions (H. Nikaido, Microbiol Mol Biol Rev 67, 593-656 (2003)), and the LAB_{WT+} peptide (SEQ ID NO: 68) (Table 2). The LAB_{Δα7} (SEQ ID NO: 2) and LAB_{T213D} (SEQ ID NO: 3) peptides which failed to bind LPS in vitro (FIGS. 8 and 9) showed no effect on bacterial cell growth irrespective of the presence or absence of EDTA.

In light of the apparent chemical-mediated sensitization, the antibacterial activity of the LAB_{WT+} peptide (SEQ ID NO: 68) was examined on different E. coli strains that may promote access to LPS. In the absence of EDTA, an MIC of 400 μM on E. coli ΔwaaD was measured (Table 2), a strain that produces a truncated LPS layer and compromised OM barrier (D. Missiakas et al., Mol Microbiol 21, 871-884 (1996)). The LAB_{WT+} peptide (SEQ ID NO: 68) also had MICs of 25 μM and 50 μM against E. coli imp4213 and E. coli FhuAΔC/Δ4L in the absence of EDTA (Table 2); where these strains are known promote the penetration of large molecules across the OM (B. A. Sampson et al., Genetics 122, 491-501 (1989); M. M. Mohammad et al., J Biol Chem 286, 8000-8013 (2011)). These findings establish the ability of the LAB_{WT+} (SEQ ID NO: 68) peptide to inhibit the growth of multiple E. coli strains.

### Example 6: Broad-Spectrum Bacterial Growth Inhibition by LAB-Peptides

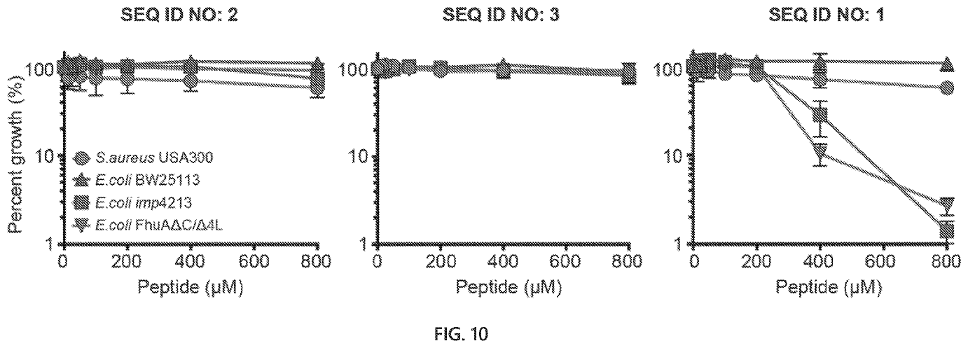

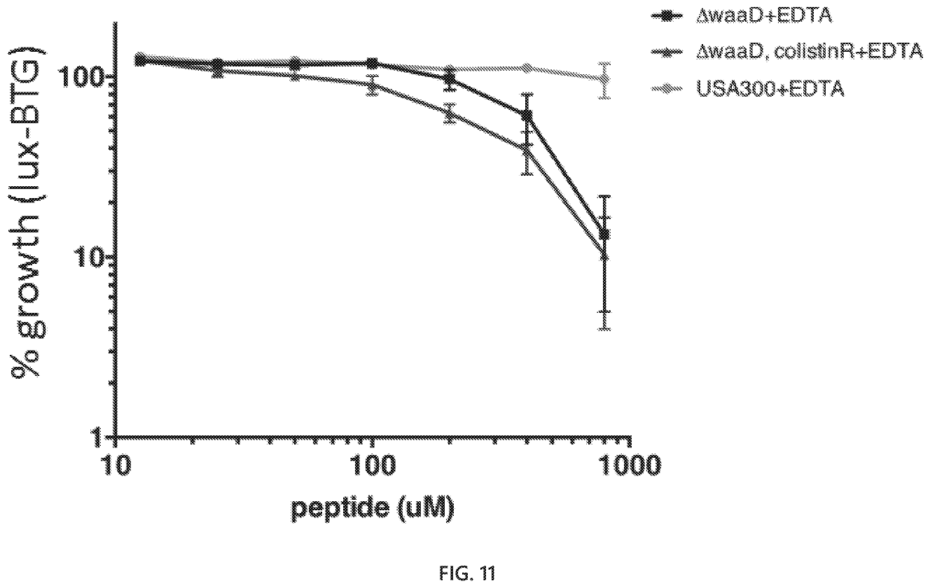

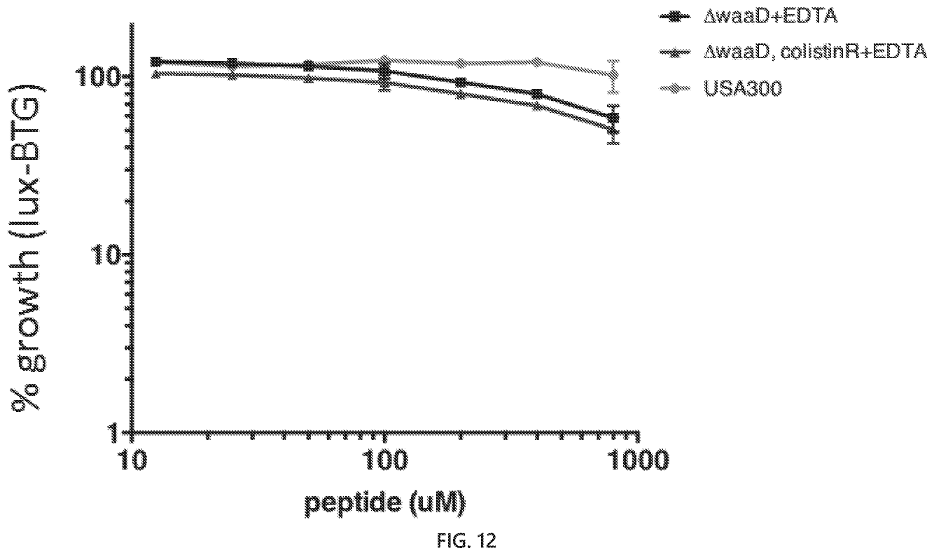

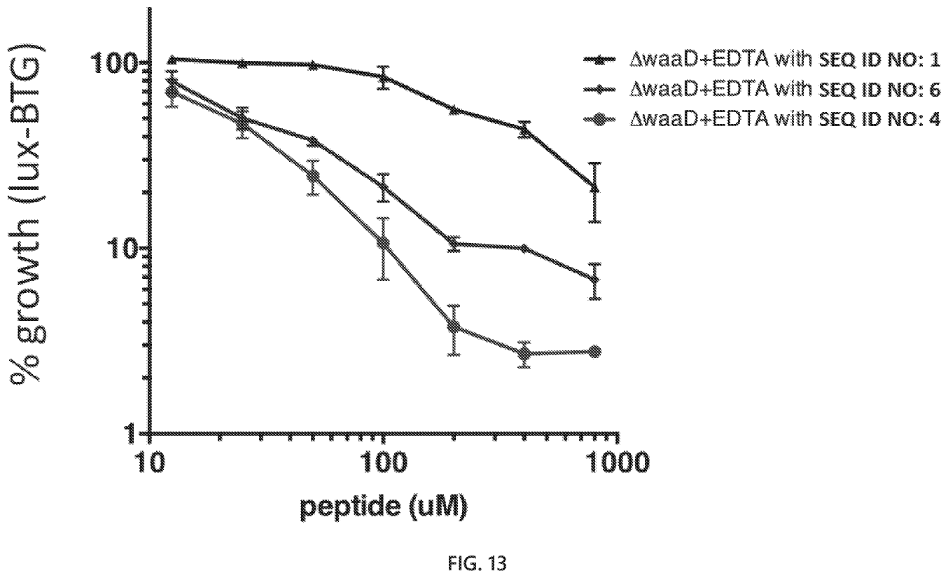

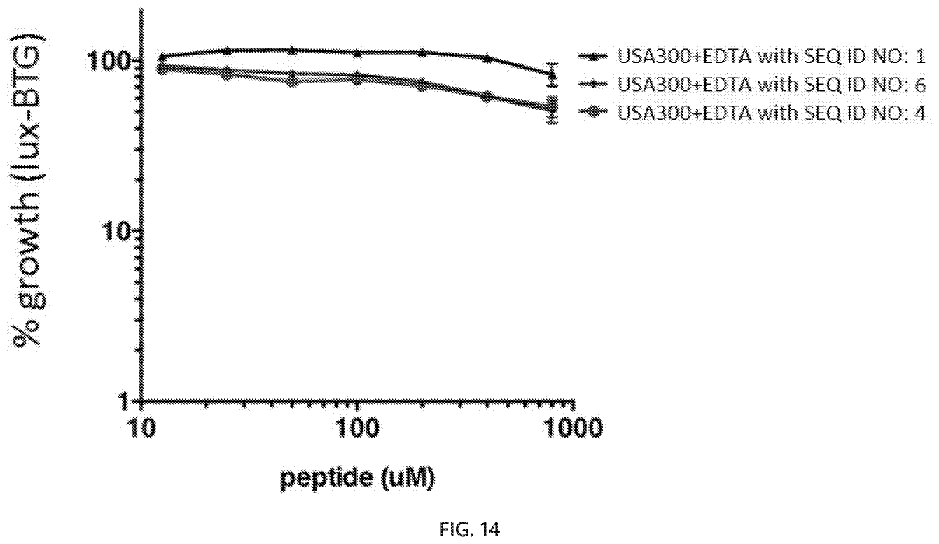

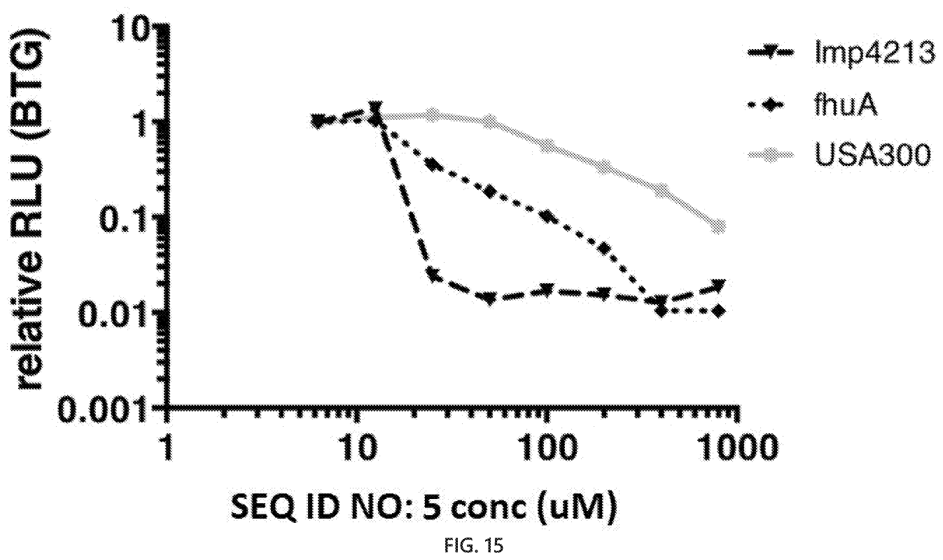

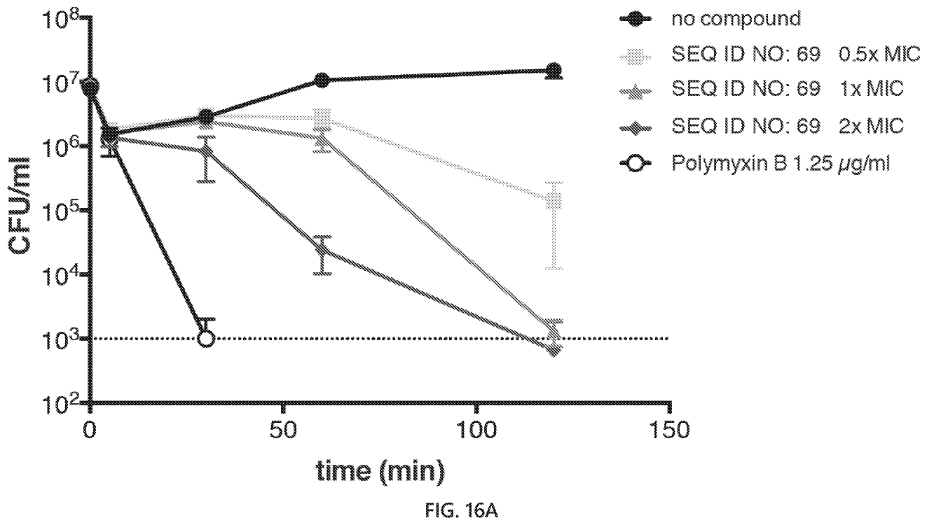

The interactions observed along the LPS-PbgA interface in the crystal structure lead directly to three testable hypotheses for the activity of the synthetic LAB_{WT+} peptide (SEQ ID NO: 68). First, all LAB peptides tested failed to impact the growth of Staphylococcus aureus (USA300), a Gram-positive bacterium which lacks LPS (FIGS. 10-12, 14, and 15; Table 2). Second, MICs of 100 μM, 200 μM, and 400 μM were measured against clinically-relevant pathogens including Enterobacter cloacae, Acinetobacter baumannii, and Pseudomonas aeruginosa in the presence of 2 mM EDTA (Table 3). These findings are in-line with the conservation of lipid A across Gram-negative bacteria and the molecular selectivity expected for a lipid A-targeting antibacterial mechanism.

TABLE 3
Exemplary MICs for SEQ ID NO: 68

| Strain | Purpose | MIC (μM) |
|---|---|---|
| E. cloacae + EDTA | Gram-negative bacterium, wild-type strain, permeabilized | 100 |
| K. pneumoniae + EDTA | Grain-negative bacterium, wild-type strain, permeabilized | >400 |
| A. baumainnii + EDTA | Gram-negative bacterium, wild-type strain, permeabilized | 400 |
| P. aeruginosa + EDTA | Gram-negative bacterium, wild-type strain, permeabilized | 400 |

Finally, two PMX-resistance determinants known to modify the phosphates on lipid A (mcr-1⁺ and pmrB^{D149Y}) were introduced and LAB_{WT+} peptide (SEQ ID NO: 68) MICs of 100 μM were measured equivalent to the parental E. coli ΔwaaD strain in the presence of EDTA (Table 4).

TABLE 4
Exemplary MICs for SEQ ID NO: 68

| Strain | Purpose | MIC (μM) |
|---|---|---|
| E. coli ΔwaaD + EDTA | Permeabilized | 100 |
| E. coli ΔwaaD pmrB^{D149Y} + EDTA | PMX-resistant, permeabilized | 100 |
| E. coli ΔwaaD MCR-1 + EDTA | PMX-resistant, permeabilized | 100 |

These results indicate that PbgA and LAB_{WT+} peptide (SEQ ID NO: 68) can bind to LPS containing PMX-resistance lipid A modifications and coordinate lipid A without depending upon positively charged side-chains (FIGS. 6 and 7). Thus SEQ ID NO: 68 has activity against a Gram-negative bacterial species and can overcome LPS modifications that impart PMX-resistance.

An A214F mutation of the LAB_{WT+} (FIGS. 6 and 7) was introduced with a (S)-2,3-diaminopropionic acid (Dap) at the T213 equivalent position. This peptide, LABv2.1 (SEQ ID NO: 69) demonstrated a MIC of 25 μM against E. coli K-12, an improvement compared to LABv2.0.

MICs of 12.5-200 μM were measured against clinically-relevant, wild-type bacterial pathogens Enterobacter cloacae, Klebsiella pneumoniae, Acinetobacter baumannii, and Pseudomonas aeruginosa for the LAB_{v2.1} peptide (Table 5). The LAB_{v2.1} peptide (SEQ ID NO: 69, YPMDapFRRFLEKWGLLR) has equivalent MICs against E. coli strains irrespective of whether PMX-resistance determinants are expressed (Table 5).

TABLE 5
Exemplary MICs for SEQ ID NO: 69

| | | MICs (μM)¹ |
|---|---|---|
| | | LAB_{v2.1} |
| | | YPMDapFRRFLEKWGLLR |
| strain² | phenotype | (SEQ ID NO: 69) |
| E. coli | WT | 50 |
| E. cloacae | WT | 12.5 |
| K. pneumoniae | WT | 100 |
| A. baumannii | WT | 12.5 |
| P. aeruginosa | WT | 200 |
| E. coli K-12 | WT | 25 |
| E. coli pmrA^{G53Y} | colistin^{R} | 12.5 |
| E. coli mcr-1 | colistin^{R} | 25 |
| E. coli imp4213 | permeable | 6.25 |
| S. aureus | WT | 400 |

¹MIC, minimal inhibitory concentration, is the lowest concentration of compound that results in complete growth inhibition. Peptides were synthesized with an N-terminal acetyl and C-terminal amide. ‘Dap’ indicates the non-natural amino acid (S)-2,3-diaminopropionic acid. See methods for details.
²The following strains were tested: E. coli-ATCC 25922 is wild-type with O-antigen, S. aureus-USA300 Gram-positive control, E. cloacae-ATCC 222 is wild-type, K. pneumoniae-ATCC 43816 is wild-type, A. baumannii-ATCC 19606 is wild-type, P. aeruginosa-PA-14 is wild-type, E. coli BW25113-a wild-type laboratory E. coli K-12 strain tacking O-antigen. E. coli pmrA^{G53Y}-BW25113 with chromosomal mutation leading to ~64-fold MIC shift for colistin under tile-tested conditions, E. coli mcr-1-BW25113 with extrachromosomal plasmid encoding mcr-1 that causes a ~64-fold MIC shift for colistin under the tested conditions.

The growth of a Gram-positive bacterium which lacks LPS, Staphylococcus aureus (USA300), was impacted only at very high concentrations of LAB_{v2.1} (Table 5). MICs of 12.5-25 μM for LAB_{v2.1} were measured when PMX-resistance determinants that modify the phospho-GlcNAc moieties of lipid A were introduced into E. coli K-12 (pmrA^{G53E} and mcr-1) (Table 5). These findings are consistent that full-length PbgA appears competent to bind modified forms of LPS (FIGS. 5-8).

TABLE 6
Activity of polymyxin B against E. coli strains with modified lipid A

| Strain¹ | Polymyxin B MIC (μM)² |
|---|---|
| E. coli | 0.2 |
| E. coli pmrA^{G53Y} | 12.5 |
| E. coli mcr-1 | 12.5 |

¹The following strains were tested: E. coli K-12 wild-type parent strain is BW25113, E. coli pmrA^{G53Y} is BW25113 with chromosomal mutation pmrA gene leading to a G53Y amino acid substitution. E. coli mcr-1 is BW25113 with extrachromosomal plasmid encoding mcr-1.
²MIC, minimal inhibitory concentration, is the lowest concentration of compound that results in complete growth inhibition. See methods for details.

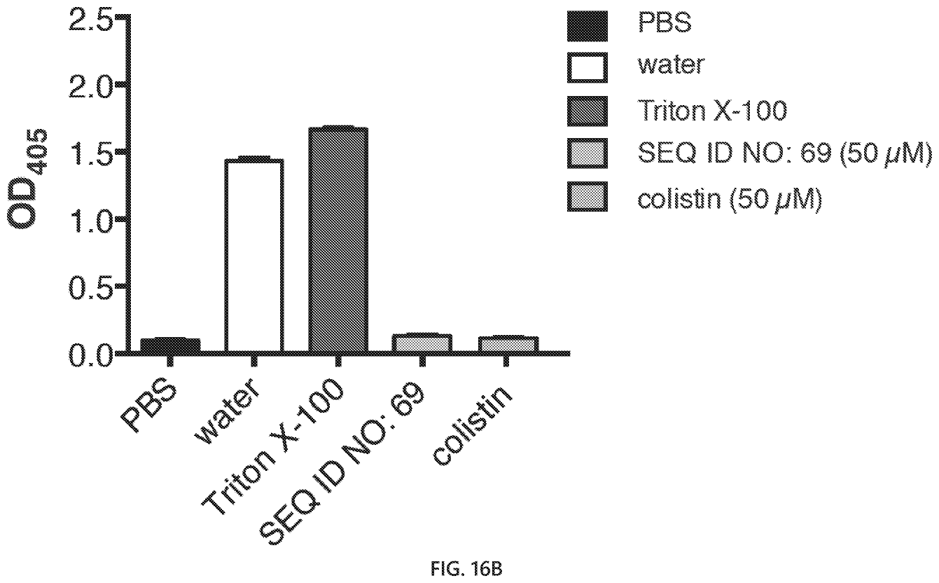

It has been established that LAB_{v2.1} peptide is bactericidal with time-kill kinetics distinct from the antibiotic polymyxin B (FIG. 16A). LAB_{v2.1} peptide (SEQ ID NO: 69) was tested in a red blood cell assay and there was no observe lysis at the 50 μM concentration tested (FIG. 16B). The instant peptides represent a new class of selective lipid A-binding peptides having activity against Gram-negative bacterial pathogens that can overcome LPS modifications which impart PMX-resistance.

### Example 7: MICs of Certain SEQ ID NOs

The following MICs shown in Table 7 were measured for exemplary SEQ ID NOs. Peptides are capped with N-terminal acetyl and C-terminal amino groups except where indicated.

TABLE 7
Exemplary peptide MIC values

| | MIC (μM) | | | | | | |
|---|---|---|---|---|---|---|---|
| | | | E coli | E. coli | E. coli | E coli | |
| SEQ ID | E. coli | E. coli | BW25113 + | ΔwaaD + | ΔwaaD + | ΔwaaD − | S. aureus |
| NO: | imp4213 | BW25113 | EDTA | EDTA | vect. | MCR1 | USA300 |
| 1 | >800 | >800 | ND | ND | ND | ND | >800 |
| 2 | >800 | >800 | ND | ND | ND | ND | >800 |
| 3 | ND | ND | ND | ND | ND | ND | ND |
| 4 | ND | ND | ND | ND | ND | ND | ND |
| 5 | 25 | >800 | 400 | 100 | ND | ND | >800 |
| 6 | 400 | >800 | 800 | >800 | ND | ND | >800 |
| 7 | 200/400 | >800 | 400 | 400 | ND | ND | >800 |
| 8 | 200 | >800 | 800 | 800 | ND | ND | >800 |
| 9 | 800 | >800 | >800 | >800 | ND | ND | >800 |
| 10 | 800 | >800 | 800 | >800 | ND | ND | >800 |
| 11 | 200 | >800 | 400 | 400 | ND | ND | >800 |
| 12 | 25 | >800 | 800 | 400 | ND | ND | >800 |
| 13 | 200 | >800 | 800 | 200 | 800 | 200 | >800 |
| 14 | 25 | 400 | ND | ND | ND | ND | 800 |
| 15 | <12.5 | ND | ND | ND | ND | ND | 800 |
| 16 | 100 | ND | ND | ND | ND | ND | >800 |
| 17 | 200 | ND | ND | ND | ND | ND | >800 |
| 18 | 200 | ND | ND | ND | ND | ND | >800 |
| 19 | 25 | ND | ND | ND | ND | ND | >800 |
| 20 | 400 | ND | ND | ND | ND | ND | >800 |
| 21 | 800 | ND | ND | ND | ND | ND | >800 |
| 22 | 400 | ND | ND | ND | ND | ND | >800 |
| 23 | 400 | ND | ND | ND | ND | ND | >800 |
| 24 | 400 | ND | ND | ND | ND | ND | >800 |
| 25 | 800 | ND | ND | ND | ND | ND | >800 |
| 26 | 800 | ND | ND | ND | ND | ND | >800 |
| 27 | 400 | ND | ND | ND | ND | ND | >800 |
| 28 | 50 | ND | ND | ND | ND | ND | >800 |
| 29 | 800 | ND | ND | ND | ND | ND | >800 |
| 30 | 800 | ND | ND | ND | ND | ND | >800 |
| 31 | 800 | ND | ND | ND | ND | ND | >800 |
| 32 | 100 | ND | ND | ND | ND | ND | >800 |
| 33 | 800 | ND | ND | ND | ND | ND | >800 |
| 34 | 25 | ND | ND | ND | ND | ND | >800 |
| 35 | 400 | ND | ND | ND | ND | ND | >800 |
| 36 | 100 | ND | ND | ND | ND | ND | >800 |
| 37 | 400 | ND | ND | ND | ND | ND | >800 |
| 38 | 100 | ND | ND | ND | ND | ND | >800 |
| 39 | 200 | ND | ND | ND | ND | ND | >800 |
| 40 | 400 | ND | ND | ND | ND | ND | >800 |
| 41 | 100 | ND | ND | ND | ND | ND | >800 |
| 42 | 800 | ND | ND | ND | ND | ND | >800 |
| 43 | 100 | ND | ND | ND | ND | ND | >800 |
| 44 | 100 | >400 | ND | ND | 200 | >800 | >800 |
| 45 | 100 | ND | ND | ND | ND | ND | >800 |
| 46 | 100 | ND | ND | ND | ND | ND | >800 |
| 47 | 200 | ND | ND | ND | ND | ND | >800 |
| 48 | 17.5 | 400 | ND | ND | 100 | 400 | >400 |
| 49 | 25 | 400 | ND | ND | 100 | 400 | >400 |
| 50 | 25 | 400 | ND | ND | 200 | 800/400 | >400 |
| 51 | 12.5 | 100 | ND | ND | 100 | 200 | >400 |
| 52 | 25 | 400 | ND | ND | 400 | 400 | >400 |
| 53 | 12.5/25 | 400 | ND | ND | 100 | 400 | >400 |
| 54 | 100 | >400 | ND | ND | 100 | >800 | >400 |
| 55 | 6.25 | 100 | ND | ND | 25 | 50 | >400 |
| 56 | 25 | >400 | ND | ND | 400 | 800 | >400 |
| 57 | 12.5 | 200 | ND | ND | 100 | 100/700 | >400 |
| 58 | 12.5 | 400 | ND | ND | 50/25 | 50 | >400 |
| 59 | 12.5 | 400 | ND | ND | 25 | 50 | >400 |
| 60 | 12.5 | 400 | ND | ND | 25 | 50 | >400 |
| 61 | 12.5 | ND | ND | ND | ND | ND | ND |
| 62 | 12.5/6.25 | 400 | ND | ND | <6.25 | 200 | ND |
| 63 | 12.5/6.25 | 400/200 | ND | ND | <6.25 | 200 | ND |
| 64 | 12.5 | 400 | ND | ND | <6.25 | 400 | ND |
| 65 | 12.5 | 200 | ND | ND | <6.25 | 100 | 800 |

ND = not determined.

As can be seen in Table 7, SEQ ID NOs: 14, 48, 49, 50, 51, 52, 53, 55, 57, 58, 59, 60, 62, 63, 64, and 65 have MICs of ≤400 μM in wild type E. coli strain BW25113.

### Example 8: MICs of Exemplary SEQ ID NOs

The following MICs shown in Table 8 were measured for exemplary SEQ ID NOs. Peptides are capped with N-terminal acetyl and C-terminal amino groups except where indicated.

TABLE 8
Exemplary MICs across species

| | MIC (μM) | | | |
|---|---|---|---|---|
| SEQ | E. coli | K. | A. | R. |
| ID NO. | BW25113 | pneumoniae | baumannii | aeruginosa |
| 44 | None | >800 | >800 | 800 |
| 48 | 400 | 400 | 200 | 400 |
| 49 | 400 | 800 | 200 | 400 |
| 50 | 400 | 800 | 400 | 800 |
| 51 | 100 | 200 | 100 | 400 |
| 52 | 400 | >800 | 200 | 400 |
| 53 | 400 | 800 | 400 | 400 |
| 54 | None | >800 | >800 | >800 |
| 55 | 100 | 400 | 25 | >800 |
| 56 | None | 800 | 400 | 800 |
| 57 | 200 | 400 | 100 | 200 |
| 58 | 400 | 400 | 25 | >800 |
| 59 | 400 | 800 | 50 | >800 |
| 60 | 400 | 800 | 25 | >800 |
| 61 | None | None | 40 | 400 |
| 62 | 400 | 200 | 200 | 200 |
| 63 | 400/200 | 200 | 200 | 400 |
| 64 | 400 | None/400 | 400 | 400 |
| 65 | 200 | 200 | 50 | 800 |
| 69 | 50 | 100 | 12.5 | 200 |
| 70 | 50 | 200 | 12.5 | 400 |
| 71 | 100 | 50 | <6.25 | 50 |
| 72 | 400 | 400 | 50 | 800 |
| 73 | 50 | 200 | 50 | 800 |
| 78 | 12.5 | 25.0 | 25.0 | 100.0 |
| 79 | 200.0 | ND | ND | ND |
| 80 | 12.5 | 12.5 | 12.5 | 25.0 |
| 81 | 12.5 | 25.0 | 25.0 | 100.0 |
| 82 | 100.0 | 50.0 | 12.5 | 50.0 |
| 83 | 200.0 | 100.0 | 50.0 | 200.0 |
| 84 | 50.0 | 50.0 | 25.0 | 200.0 |
| 85 | 200.0 | 200.0 | 100.0 | 200.0 |
| 86 | 25.0 | 50.0 | 25.0 | 50.0 |
| 87 | 100.0 | 25.0 | 25.0 | 50.0 |
| 88 | 100.0 | 25.0 | 12.5 | 25.0 |
| 89 | 25.0 | 25.0 | 25.0 | 100.0 |
| 90 | 200.0 | 50.0 | 6.25 | 25.0 |
| 91 | 50.0 | >400.0 | 50.0 | >400.0 |
| 92 | 200.0 | >400.0 | 200.0 | >400.0 |
| 93 | 200.0 | 400.0 | 50.0 | >400.0 |
| 94 | 200.0 | >400.0 | 200.0 | >400.0 |
| 95 | 50.0 | ND | ND | ND |
| 96 | 50.0 | ND | ND | ND |
| 97 | 200.0 | ND | ND | ND |
| 98 | 25.0 | ND | ND | ND |
| 99 | 200.0 | ND | ND | ND |
| 100 | 25.0 | ND | ND | ND |
| 101 | 50.0 | ND | ND | ND |
| 102 | 25.0 | ND | ND | ND |
| 103 | <12.5 | ND | ND | ND |
| 104 | 200 | ND | ND | ND |
| 105 | 50 | ND | ND | ND |
| 106 | <12.5 | ND | ND | ND |
| 107 | 12.5 | ND | ND | ND |
| 108 | <12.5 | ND | ND | ND |
| 200 | 50.0 | 200.0 | 50.0 | 50.0 |
| 201 | 25.0 | >400.0 | >400.0 | >400.0 |
| 202 | 50.0 | >400.0 | >400.0 | >400.0 |
| 203 | 200.0 | >400.0 | 400.0 | >400.0 |
| 204 | 200.0 | ND | ND | ND |
| 205 | 200.0 | ND | ND | ND |
| 206 | 200.0 | 200.0 | 200.0 | 200.0 |
| 207 | 100.0 | 100.0 | 100.0 | 100.0 |
| 208 | 100.0 | 200.0 | 200.0 | 200.0 |
| 209 | 6.25 | 12.5 | 12.5 | 25.0 |
| 210 | 200.0 | >400.0 | >400.0 | >400.0 |
| 211 | 50.0 | >400.0 | >400.0 | >400.0 |
| 212 | 100.0 | >400.0 | 200.0 | >400.0 |
| 213 | 12.5 | ND | ND | ND |
| 214 | 100.0 | >400.0 | >400.0 | >400.0 |
| 215 | 200.0 | >400.0 | >400.0 | >400.0 |
| 216 | 100.0 | >400.0 | >400.0 | >400.0 |
| 217 | 12.5 | ND | ND | ND |
| 218 | 200.0 | ND | ND | ND |
| 219 | 25.0 | ND | ND | ND |
| 220 | 100.0 | ND | ND | ND |
| 221 | 200.0 | ND | ND | ND |
| 222 | 12.5 | ND | ND | ND |
| 223 | 100.0 | ND | ND | ND |
| 224 | 100.0 | ND | ND | ND |
| 225 | 100.0 | ND | ND | ND |
| 226 | 25.0 | ND | ND | ND |
| 227 | 50.0 | ND | ND | ND |

The present invention leverages the lipid A-binding (LAB) motif discovered in PbgA and demonstrates that free LAB peptides inhibit bacterial cell growth. While this work reveals important insight into LPS perception within the IM of Gram-negative bacteria, it also identifies a new class of antimicrobial peptide capable of inhibiting diverse strains of Grami-negative bacteria, including strains that are resistant to PMXs, our present-day antibiotics of last resort. Additionally, two new antibiotic strategies have been identified: (1) disrupting the LPS-PbgA interface can potentiate the access of antibiotics across the OM in E. coli; and (2) the unanticipated lipid A-binding motif in PbgA identified herein can be leveraged to produce synthetic peptides such as those provided herein that are capable of selectively binding to LPS. Growth inhibition of E. coli. E. cloacae, A. baumannii, and P. aeruginosa strains have been observed as provided herein.

Although the foregoing invention has been described in some detail by way of illustration and example for purposes of clarity of understanding, the descriptions and examples should not be construed as limiting the scope described herein. The disclosures of all patent and scientific literature cited herein are expressly incorporated in their entirety by reference.

What is claimed is:

1. A peptide of Formula I:
R¹—X¹-Pro-X²—X³—X⁴—X⁵-Arg-X⁶-Leu-X⁷-Lys-X⁸-Gly-Leu-Leu-Arg-R²(SEQ ID NO: 66) (Formula I)
wherein,
R¹ is acetyl or is absent;
X¹, X², X³, X⁴, X⁵, X⁶, and X⁷ are each independently a natural or non-natural amino acid residue;
X⁸ is tryptophan or histidine; and
R² is amino or is absent.

2. A peptide of Formula II:
R¹—X¹-Pro-X²—X³—X⁴—X⁵-Arg-X⁶-Leu-X⁷-Lys-X⁸-Gly-Leu-R²(SEQ ID NO: 74); (Formula II)
wherein,
R¹ is acetyl or is absent;
X¹, X², X³, X⁴, X⁵, X⁶, and X⁷ are each independently a natural or non-natural amino acid residue;
X⁸ is tryptophan or histidine; and
R² is amino or is absent.

3. The peptide of any one of claims 1-2, wherein X¹ is tyrosine, lysine, alanine, phenylalanine, tryptophan, or arginine.

4. The peptide of any one of claims 1-3, wherein X¹ is tyrosine or lysine.

5. The peptide of any one of claims 1-4, wherein X² is methionine, N-methylmethionine, norleucine, alanine, leucine, phenylalanine, N-methylphenylalanine, homophenylalanine, (S)-2,3-diaminopropionic acid, or tryptophan.

6. The peptide of any one of claims 1-5, wherein X³ is threonine, allo-threonine, serine, asparagine, (S)-2,3-diaminopropionic acid, (S)-2,3-diaminobutyric acid, homoserine, lysine, arginine, or alanine.

7. The peptide of any one of claims 1-6, wherein X⁴ is alanine, 2-aminobutyric acid, methionine, N-methylmethionine, phenylalanine, N-methylphenylalanine, N-methylalanine, tyrosine, (S)-2-aminoheptanoic acid, 2-amino-2-methylpropanoic acid, (S)-2,3-diaminopropionic acid, tryptophan, or arginine.

8. The peptide of any one of claims 1-7, wherein X⁵ is arginine, ornithine, glutamine, lysine, or (S)-2,3-diaminopropionic acid.

9. The peptide of any one of claims 1-8, wherein X⁶ is phenylalanine, homophenylalanine, (S)-3-([1,1′-biphenyl]-4-yl)-2-aminopropanoic acid, (S)-2-amino-2-(naphthalen-1-yl)acetic acid, tyrosine, or tryptophan.

10. The peptide of any one of claims 1-9, wherein X⁷ is glutamic acid, glutamine, alanine, serine, homoserine, or arginine.

11. The peptide of any one of claims 1-10, wherein X⁸ is tryptophan.

12. The peptide of any one of claims 1-10, wherein X⁸ is histidine.

13. The peptide of any one of claims 1-12, wherein:
R¹ is acetyl or is absent;
X¹ is tyrosine, lysine, alanine, phenylalanine, tryptophan, or arginine;
X² is methionine, N-methylmethionine, norleucine, alanine, leucine, phenylalanine, N-methylphenylalanine, homophenylalanine, (S)-2,3-diaminopropionic acid, or tryptophan;
X³ is threonine, allo-threonine, serine, asparagine, (S)-2,3-diaminopropionic acid, (S)-2,3-diaminobutyric acid, homoserine, lysine, arginine, or alanine;
X⁴ is alanine, 2-aminobutyric acid, methionine, N-methylmethionine, phenylalanine, N-methylphenylalanine, N-methylalanine, tyrosine, (S)-2-aminoheptanoic acid, 2-amino-2-methylpropanoic acid, (S)-2,3-diaminopropionic acid, tryptophan, or arginine;
X⁵ is arginine, ornithine, glutamine, 2-amino-2-methylpropanoic acid, (S)-2,3-diaminopropionic acid, or lysine;
X⁶ is phenylalanine, homophenylalanine, (S)-3-([1,1′-biphenyl]-4-yl)-2-aminopropanoic acid, (S)-2-amino-2-(naphthalen-1-yl)acetic acid, tyrosine, or tryptophan;
X⁷ is glutamic acid, glutamine, alanine, serine, homoserine, or arginine;
X⁸ is tryptophan or histidine; and
R² is amino or is absent.

14. A peptide of Formula III:
R¹-Arg-X^{a}—X^{b}—X^{c}—X^{d}-Arg-Arg-X^{e}-Leu-X^{f}—X^{g}—X^{h}-Gly-Leu-R²(SEQ ID NO: 228) (Formula III)
wherein,
R¹ is acetyl or is absent;
X^{a}, X^{b}, X^{c}, X^{d}, X^{e}, X^{f}, X^{g}, and X^{h} are each independently a natural or non-natural amino acid residue; and
R² is amino or is absent.

15. The peptide of claim 14, wherein X is proline or (S)-piperidine-2-carboxylic acid.

16. The peptide of claim 14 or 15, wherein X^{b} is methionine, N-methylmethionine, homophenylalanine or (S)-2-amino-5-phenylpentanoic acid.

17. The peptide of any one of claims 14 to 16, wherein X^{c} is threonine or (S)-2,3-diaminopropionic acid.

18. The peptide of any one of claims 14 to 17, wherein X^{d} is tryptophan, alanine, serine, methionine, (S)-2-aminoheptanoic acid, (S)-3-([1,1′-biphenyl]-4-yl)-2-aminopropanoic acid, O-methyl-L-serine, N-methylalanine, or 2-amino-2-methylpropanoic acid.

19. The peptide of any one of claims 14 to 18, X^{d} is tryptophan.

20. The peptide of any one of claims 14 to 19, wherein X^{e} is phenylalanine, tryptophan, (S)-3-([1,1′-biphenyl]-4-yl)-2-aminopropanoic acid, or (S)-2-amino-2-(naphthalen-1-yl)acetic acid.

21. The peptide of any one of claims 14 to 20, wherein X^{f} is alanine, glutamic acid, or homoserine.

22. The peptide of any one of claims 14 to 21, wherein X^{g} is lysine or (S)-2,3-diaminobutyric acid.

23. The peptide of any one of claims 14 to 22, wherein X^{h} is arginine, tyrosine, histidine, tryptophan, (S)-2,3-diaminobutyric acid, or (S)-2-aminoheptanoic acid.

24. The peptide of claim 14, wherein:
R¹ is acetyl or is absent;
X^{a} is proline or (S)-piperidine-2-carboxylic acid;
X^{b} is methionine, N-methylmethionine, homophenylalanine or (S)-2-amino-5-phenylpentanoic acid;
X^{c} is threonine or (S)-2,3-diaminopropionic acid;
X^{d} is tryptophan, alanine, serine, methionine, (S)-2-aminoheptanoic acid, (S)-3-([1,1′-biphenyl]-4-yl)-2-aminopropanoic acid, O-methyl-L-serine, N-methylalanine, or 2-amino-2-methylpropanoic acid;
X^{e} is phenylalanine, tryptophan, (S)-3-([1,1′-biphenyl]-4-yl)-2-aminopropanoic acid, or (S)-2-amino-2-(naphthalen-1-yl)acetic acid;
X^{f} is alanine, glutamic acid, or homoserine;
X^{g} is lysine or (S)-2,3-diaminobutyric acid;
X^{h} is Arg, tyrosine, histidine, tryptophan, (S)-2,3-diaminobutyric acid, or (S)-2-aminoheptanoic acid; and
R² is amino or is absent

25. The peptide of any one of claims 1 to 24, wherein R¹ is acetyl.

26. The peptide of any one of claims 1-25, wherein R² is amino.

27. A peptide comprising an amino acid corresponding to one of SEQ ID NOs: 5-65, 68-73, or 78-108.

28. A peptide comprising an amino acid corresponding to one of SEQ ID NOs: 200-227.

29. The peptide of any one of claims 1-28, wherein the peptide binds to a lipopolysaccharide.

30. The peptide of claim 29, wherein the peptide binds to the lipid A portion of a lipopolysaccharide.

31. The peptide of claim 28 or 29, wherein the peptide has a lipopolysaccharide-binding affinity in terms of Kd of ≤100 μM as measured by biolayer interferometry.

32. A pharmaceutical composition comprising a peptide of any one of claims 1-28, and a pharmaceutically acceptable excipient.

33. The pharmaceutical composition of claim 32, further comprising an additional therapeutic agent.

34. The pharmaceutical composition of claim 33, wherein the additional therapeutic agent comprises antibiotics or antiseptics.

35. A peptide of any one of claims 1-28, for use as therapeutically active substance.

36. A use of a peptide of any one of claims 1-28, for the therapeutic treatment of a bacterial infection.

37. A use of a peptide of any one of claims 1-28, for the preparation of a medicament for the therapeutic treatment of a bacterial infection.

38. A peptide of any one of claims 1-28, for the therapeutic treatment of a bacterial infection.

39. A method for the therapeutic treatment of a bacterial infection, which method comprises administering a therapeutically effective amount of a peptide of any one of claims 1-28.

40. The method of claim 39, further comprising administering an additional therapeutic agent.

41. The method of claim 40, wherein the additional therapeutic agent comprises antibiotics or antiseptics.

42. The use of claim 36 or 37, or the peptide of claim 38, or the method of any one of claims 39-41, wherein the bacterial infection is caused by a Gram-negative bacterium.

43. The use of claim 36 or 37, or the peptide of claim 38, or the method of any one of claims 39-41, wherein the bacterial infection is caused by a Gram-negative bacterium selected from the group consisting of Escherichia col, Klebsiella spp., Pseudomonas spp., Enterobacter spp., Bordatella spp., Burkholderia sp., Stenotrophomonas maltophiha, Bacteroides spp., Campylobacter spp., Francisella tularensis, Helicobacter pylori, Legionella spp., and Vibrio spp.

44. The use of claim 36 or 37, or the peptide of claim 38, or the method of any one of claims 39-41, wherein the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

45. The peptide of any one of claims 1-28, conjugated to a therapeutic agent.

46. The peptide of any one of claims 1-28, conjugated to a label.

47. The peptide of claim 46, wherein the label is a radioisotope, a fluorescent dye, or an enzyme.

48. A method of producing the peptide of any one of claims 1-28, comprising chemically synthesizing the peptide.

49. An isolated nucleic acid encoding the peptide of any one of claims 1-28.

50. An expression vector encoding the nucleic acid molecule of claim 49.

51. A cell comprising the expression vector of claim 50.

52. A method of producing the peptide of any one of claims 1-28, comprising culturing the cell of claim 51 and recovering the peptide from the cell culture.

53. A method of treating an individual having a bacterial infection comprising administering to the individual an effective amount of a peptide that binds to a lipopolysaccharide comprising an amino acid sequence having a homology of 2 50% with SEQ ID NO: 1.

54. The method of claim 53, wherein the peptide binds to a lipopolysaccharide of a Gram-negative bacterium.

55. The method of claim 54, wherein the Gram-negative bacterium is selected from the group consisting of Escherichia coli, Klebsiella spp., Pseudomonas spp., Enterobacter spp., Bordatella spp., Burkholderia sp., Stenotrophomonas maltophiha, Bacteroides spp., Campylobacter spp., Francisella tularensis, Helicobacter pylori, Legionella spp., and Vibrio spp.

56. The method of any one of claims 53-55, wherein the peptide binds to the lipid A portion of a lipopolysaccharide.

57. The method of any one of claims 53-55, wherein the peptide has a lipopolysaccharide-binding affinity in terms of Kd of ≤100 μM as measured by biolayer interferometry.

58. The method of any one of claims 53-57, wherein the peptide binds to a lipopolysaccharide selectively over a bacterial membrane phospholipid.

59. The method of any one of claims 53-58, wherein the peptide has an IC₅₀ of ≤10 μM against a Gram-negative bacterium, as measured by an in vitro bacterial growth assay in LB or Mueller Hinton II cation-adjusted broth at 37° C.

60. The method of any one of claims 53-58, wherein the peptide has an MIC of ≤500 μM against a Gram-negative bacterium, as measured by an in vitro bacterial growth assay in LB or Mueller Hinton II cation-adjusted broth at 37° C.

61. The method of any one of claims 53-60, wherein the peptide has a length of 10-20 amino acid residues.

62. The method of any one of claims 53-61, wherein the peptide comprises an amino acid sequence having a homology of: ≥60%, ≥70%, ≥80%, ≥90%, or ≥95% with SEQ ID NO: 1.

63. The method of any one of claims 53-62, wherein the individual is human.

64. The method of claim 63, wherein the bacterial infection is selected from the group consisting of a respiratory tract infection, a lung infection, an upper respiratory tract infection, a lower respiratory tract infection, a nasopharyngeal infection, a urinary tract infection, a complicated urinary tract infection, pneumonia, nosocomial pneumonia, community-acquired pneumonia, hospital-acquired pneumonia, ventilator associated pneumonia, bacteremia, a bloodstream infection, central line associated bloodstream infection, intra-abdominal infection, intra-abdominal infection, skin and soft tissue infection, complicated skin and soft tissue infection, surgical site infection, complicated surgical site infection, skin and skin structure infection, complicated skin and skin structure infection, osteomyelitis, prosthetic joint infection, and post-operative infection.

65. The invention as hereinbefore described.
